SEQUENCE LISTING (5 sequences)

SEQ ID NO: 1 - DNA, 279 bp, Human
  misc_feature (1)..(279): CCL3
atgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctga

SEQ ID NO: 2 - protein, 92 aa, Human
  MISC_FEATURE (1)..(92): CCL3
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 3 - DNA, 279 bp, Human
  misc_feature (1)..(279): CCL4
atgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactga

SEQ ID NO: 4 - protein, 92 aa, Human
  MISC_FEATURE (1)..(92): CCL4
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 5 - protein, 15 aa, Artificial Sequence
  Peptide Substrate
AVLESEEELYSSARQ

(19) United States
(12) Patent Application Publication
(10) Pub. No.: US 2015/0238490 A1
(43) Pub. Date: Aug. 27, 2015

(54) BIOMARKERS FOR PREDICTING RESPONSE OF DLBCL TO TREATMENT WITH IBRUTINIB

(71) Applicant: Pharmacyclics, Inc., Sunnyvale, CA (US)

(72) Inventor: Jan A. BURGER, Houston, TX (US)

(21) Appl. No.: 14/628,112

(22) Filed: Feb. 20, 2015

Related U.S. Application Data
(60) Provisional application No. 61/943,260, filed on Feb. 21, 2014.

(51) Int. Cl. A61K 31/519; C12Q 1/68; G01N 33/574

(52) U.S. Cl. CPC A61K 31/519; G01N 33/57426; C12Q 1/6886; C12Q 2600/158; C12Q 2600/106; G01N 2333/523; G01N 2800/52

(57) ABSTRACT

Described herein are diagnostic methods for selecting patients diagnosed with diffuse large B-cell lymphoma (DLBCL) for treatment with an inhibitor of Bruton's tyrosine kinase (BTK) based on the level of expression the biomarkers CCL3 and/or CCL4. Also provided are methods for identifying DLBCL patients likely to respond to treatment with a BTK inhibitor and for evaluating treatment of DLBCL with a BTK inhibitor. Methods of treating a patient also are provided. Also provided are compositions, combinations, and kits.

### CROSS-REFERENCE

This application claims the benefit of U.S. Provisional Application No. 61/943,260, filed Feb. 21, 2014, which application is incorporated herein by reference.

### STATEMENT AS TO FEDERALLY SPONSORED RESEARCH

The invention disclosed herein was made, at least in part, with U.S. government support under Grant No. P50CA136411 by the National Institutes of Health. Accordingly, the U.S. Government has certain rights in this invention.

### INCORPORATION BY REFERENCE OF SEQUENCE LISTING SUBMITTED AS A TEXT FILE VIA EFS-WEB

The instant application contains a Sequence Listing, which has been submitted as a computer readable text file in ASCII format via EFS-Web and is hereby incorporated in its entirety by reference herein. The text file, created date of Feb. 12, 2014, is named 25922-305-201SEQ.txt and is 3,365 bytes in size.

### BACKGROUND OF THE INVENTION

Diffuse large B cell lymphoma (DLBCL) is the most prevalent type of aggressive non-Hodgkin's lymphoma (NHL) in the United States. The ABC subtype of DLBCL (ABC-DLBCL) accounts for approximately 30% total DLBCL diagnoses. While majority of the patients with DLBCL show response to the initial treatment, approximately one-third of patients have refractory disease or experience relapse after the standard therapies. B cell receptor (BCR) signaling is an important growth and survival pathway in various B cell malignancies, including DLBCL.

### SUMMARY OF THE INVENTION

Disclosed herein, in certain embodiments, are methods for selecting patients diagnosed with diffuse large B-cell lymphoma (DLBCL) for treatment with an inhibitor of Bruton's tyrosine kinase (BTK) based on the level of expression the biomarkers CCL3 and/or CCL4. In some embodiments, the methods comprise identifying patient likely to respond to treatment with a BTK inhibitor. In some embodiments, the methods comprise determining a treatment regimen. Also disclosed herein, in certain embodiments, are methods for evaluating treatment of diffuse large B-cell lymphoma (DLBCL), in an individual in need thereof, comprising: administering to the individual a therapeutically effective amount of an inhibitor of a BTK inhibitor and determining the responsiveness of the patient to treatment based on the level of expression of CCL3 and/or CCL4. Also disclosed herein, in certain embodiments, are methods for treatment of diffuse large B-cell lymphoma (DLBCL), in an individual in need thereof, comprising: administering to the individual a therapeutically effective amount of an inhibitor of a BTK inhibitor, determining the responsiveness of the patient to treatment based on the level of expression of CCL3 and/or CCL4, and continuing treatment if the level of CCL3 and/or CCL4 expression has decreased by a predetermined amount. Also disclosed herein, in certain embodiments, are methods for treatment of diffuse large B-cell lymphoma (DLBCL), in an individual in need thereof, comprising: administering to the individual a therapeutically effective amount of an inhibitor of a BTK inhibitor, determining the responsiveness of the patient to treatment based on the level of expression of CCL3 and/or CCL4, and discontinuing treatment if the level of CCL3 and/or CCL4 expression has not decreased by a predetermined amount. As demonstrated herein, high levels of expression of CCL3 and/or CCL4 prior to treatment are predictive of a therapeutic response to treatment with a BTK inhibitor (e.g., ibrutinib). In addition, a decrease in levels of CCL3 and/or CCL4 expression (e.g., normalization of expression) following administration with a BTK inhibitor (e.g., ibrutinib) is predictive of the efficacy of the BTK inhibitor for treatment of DLBCL.

Described herein, in certain embodiments, are methods for determining whether a patient diagnosed with diffuse large B cell lymphoma (DLBCL) is likely to respond to therapy with a BTK inhibitor comprising: (a) detecting the expression of CCL3 and/or CCL4 in a sample from the patient; and (b) characterizing the patient as likely to respond to therapy with a therapeutically effective amount of a BTK inhibitor if the level of expression of CCL3 and/or CCL4 in the sample is elevated compared to a control. In some embodiments, the methods further comprise administering a therapeutically effective amount of a BTK inhibitor to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining whether a patient diagnosed with diffuse large B cell lymphoma (DLBCL) is likely to respond to therapy with ibrutinib comprising: (a) detecting the expression of CCL3 and/or CCL4 in a sample from the patient; and (b) characterizing the patient as likely to respond to therapy with a therapeutically effective amount of ibrutinib if the level of expression of CCL3 and/or CCL4 in the sample is elevated compared to a control. In some embodiments, the methods further comprise administering a therapeutically effective amount of ibrutinib to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining whether a patient diagnosed with diffuse large B cell lymphoma (DLBCL) is likely to respond to therapy with a BTK inhibitor comprising: (a) detecting the expression of CCL3 and/or CCL4 in a sample from the patient; and (b) characterizing the patient as likely to respond to therapy with a therapeutically effective amount of a BTK inhibitor if the level of expression of CCL3 and/or CCL4 in the sample is elevated compared to a control. In some embodiments, the methods further comprise administering a therapeutically effective amount of a BTK inhibitor to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining cancer prognosis in a patient diagnosed with DLBCL comprising: (a) detecting the expression of CCL3 and/or CCL4 in a sample from the patient; and (b) characterizing the patient as likely to have increased overall survival (OS) and/or progression free survival when the patient is treated with a therapeutically effective amount of ibrutinib if the level of expression of CCL3 and/or CCL4 in the sample is elevated relative to a control compared to a patient not having elevated CCL3 and/or CCL4 expression. In some embodiments, the methods further comprise administering a therapeutically effective amount of ibrutinib to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining cancer prognosis in a patient diagnosed with DLBCL comprising: (a) detecting the expression of CCL3 and/or CCL4 in a sample from the patient; and (b) characterizing the patient as likely to have increased overall survival (OS) and/or progression free survival when the patient is treated with a therapeutically effective amount of a BTK inhibitor if the level of expression of CCL3 and/or CCL4 in the sample is elevated relative to a control compared to a patient not having elevated CCL3 and/or CCL4 expression. In some embodiments, the methods further comprise administering a therapeutically effective amount of a BTK inhibitor to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining whether a patient diagnosed with diffuse large B cell lymphoma (DLBCL) is likely to respond to therapy with a therapeutically effective amount of ibrutinib comprising: (a) detecting the expression of CCL3 and/or CCL4 in a sample from the patient following administration of a therapeutically effective amount of ibrutinib to the patient; and (b) characterizing the patient as likely to respond to therapy with ibrutinib if the level of expression of CCL3 and/or CCL4 in the sample is decreased compared to the level of CCL3 and/or CCL4 expression prior to treatment with ibrutinib. In some embodiments, the level of CCL3 and/or CCL4 expression decreases by 5%, 10%, 15%, 20%, 25%, 30%, 35%, 40%, 45%, 50%, 55%, 60%, 65%, 70%, 75%, 80%, 85%, 90%, 95% 99% or greater following treatment with ibrutinib. In some embodiments, the level of CCL3 and/or CCL4 expression following treatment with ibrutinib decreases to the level of expression of CCL3 and/or CCL4 in a human that does not have DLBCL. In some embodiments, the methods further comprise administering an additional therapeutically effective amount of ibrutinib to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for determining whether a patient diagnosed with diffuse large B cell lymphoma (DLBCL) is likely to respond to therapy with a therapeutically effective amount of a BTK inhibitor comprising: (a) detecting the expression of CCL3 and/or CCL4 in a sample from the patient following administration of a therapeutically effective amount of a BTK inhibitor to the patient; and (b) characterizing the patient as likely to respond to therapy with the BTK inhibitor if the level of expression of CCL3 and/or CCL4 in the sample is decreased compared to the level of CCL3 and/or CCL4 expression prior to treatment with the BTK inhibitor. In some embodiments, the level of CCL3 and/or CCL4 expression decreases by % 5%, 10%, 15%, 20%, 25%, 30%, 35%, 40%, 45%, 50%, 55%, 60%, 65%, 70%, 75%, 80%, 85%, 90%, 95% 99% or greater following treatment with the BTK inhibitor. In some embodiments, the level of CCL3 and/or CCL4 expression following treatment with the BTK inhibitor decreases to the level of expression of CCL3 and/or CCL4 in a human that does not have DLBCL. In some embodiments, the methods further comprise administering an additional therapeutically effective amount of the BTK inhibitor to the patient following step (b). In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in some embodiments, are methods for determining whether a patient diagnosed with diffuse large B cell lymphoma (DLBCL) is likely to respond to therapy with a BTK inhibitor comprising: determining a pre-administration expression level of CCL3 and/or CCL4 in a sample from the patient; administering a dose of a BTK inhibitor; detecting a post-administration expression level of CCL3 and/or CCL4 in a sample from the patient following administration of BTK inhibitor to the patient; and characterizing the patient as likely to respond to therapy with BTK inhibitor if the post-administration expression level of CCL3 and/or CCL4 decreased compared to the pre-administration level of CCL3 and/or CCL4. In some embodiments, the dose of BTK inhibitor is about 140 mg to about 840 mg. In some embodiments, the dose is selected from the group consisting of 140 mg, 280 mg, 420 mg, 560 mg, 700 mg, or 840 mg. In some embodiments, the level of CCL3 and/or a CCL4 expression decreases by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of BTK inhibitor. In some embodiments, the post-administration level of CCL3 and/or CCL4 decreases to the expression level of CCL3 and/or CCL4 in a human that does not have DLBCL. In some embodiments, the post-administration expression level of CCL3 and/or CCL4 is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following administration of BTK inhibitor. In some embodiments, the method further comprises administering a therapeutically effective amount of BTK inhibitor to the patient. In some embodiments, the sample is a blood sample or a serum sample. In some embodiments, the determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the measuring the amount of CCL3 and/or CCL4 protein is with an enzyme-linked immunosorbent assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is activated B cell-like (ABC) subtype of DLBCL. In some embodiments, the ABC-DLBCL is characterized by a CD79B mutation. In some embodiments, the CD79B mutation is a mutation of the immunoreceptor tyrosine-based activation motif (ITAM) signaling module. In some embodiments, the CD79B mutation is a missense mutation of the first immunoreceptor tyrosine-based activation motif (ITAM) tyrosine. In some embodiments, the CD79B mutation increases surface BCR expression and attenuates Lyn kinase activity. In some embodiments, the ABC-DLBCL is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the ABC-DLBCL is characterized by a mutation in MyD88, A20, or a combination thereof. In some embodiments, the MyD88 mutation is the amino acid substitution L265P in the MYD88 Toll/IL-1 receptor (TIR) domain. In some embodiments, the methods further comprise administering an additional anticancer agent.

Described herein, in some embodiments, are methods for determining whether a patient diagnosed with diffuse large B cell lymphoma (DLBCL) is likely to respond to therapy with ibrutinib comprising: determining a pre-administration expression level of CCL3 and/or CCL4 in a sample from the patient; administering a dose of ibrutinib; detecting a post-administration expression level of CCL3 and/or CCL4 in a sample from the patient following administration of ibrutinib to the patient; and d) characterizing the patient as likely to respond to therapy with ibrutinib if the post-administration expression level of CCL3 and/or CCL4 decreased compared to the pre-administration level of CCL3 and/or CCL4. In some embodiments, the dose of ibrutinib is about 140 mg to about 840 mg. In some embodiments, the dose is selected from the group consisting of 140 mg, 280 mg, 420 mg, 560 mg, 700 mg, or 840 mg. In some embodiments, the level of CCL3 and/or a CCL4 expression decreases by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of ibrutinib. In some embodiments, the post-administration level of CCL3 and/or CCL4 decreases to the expression level of CCL3 and/or CCL4 in a human that does not have DLBCL. In some embodiments, the post-administration expression level of CCL3 and/or CCL4 is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following administration of ibrutinib. In some embodiments, the method further comprises administering a therapeutically effective amount of ibrutinib to the patient. In some embodiments, the sample is a blood sample or a serum sample. In some embodiments, the determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the measuring the amount of CCL3 and/or CCL4 protein is with an enzyme-linked immunosorbent assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is activated B cell-like (ABC) subtype of DLBCL. In some embodiments, the ABC-DLBCL is characterized by a CD79B mutation. In some embodiments, the CD79B mutation is a mutation of the immunoreceptor tyrosine-based activation motif (ITAM) signaling module. In some embodiments, the CD79B mutation is a missense mutation of the first immunoreceptor tyrosine-based activation motif (ITAM) tyrosine. In some embodiments, the CD79B mutation increases surface BCR expression and attenuates Lyn kinase activity. In some embodiments, the ABC-DLBCL is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the ABC-DLBCL is characterized by a mutation in MyD88, A20, or a combination thereof. In some embodiments, the MyD88 mutation is the amino acid substitution L265P in the MYD88 Toll/IL-1 receptor (TIR) domain. In some embodiments, the methods further comprise administering an additional anticancer agent.

Described herein, in certain embodiments, are methods for treating DLBCL in a patient in need thereof comprising: (a) determining an expression level of CCL3 and/or CCL4 in a sample from the patient from the patient prior to administration of ibrutinib; and (b) administering to the patient a therapeutically effective amount of ibrutinib if the expression of CCL3 and/or CCL4 is increased relative to a control or reference level. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for treating DLBCL in a patient in need thereof comprising: (a) determining an expression level of CCL3 and/or CCL4 in a sample from the patient from the patient prior to administration of a BTK inhibitor; and (b) administering to the patient a therapeutically effective amount of the BTK inhibitor if the expression of CCL3 and/or CCL4 is increased relative to a control or reference level. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for optimizing the treatment of DLBCL in a patient in need thereof comprising: (a) determining an expression level of CCL3 and/or CCL4 in a sample from the patient following administration of a therapeutically effective amount of ibrutinib; and (b) modifying, discontinuing, or continuing the treatment based on the expression of CCL3 and/or CCL4 relative to a control or reference level. In some embodiments, the methods comprise increasing or decreasing the dosage of ibrutinib. In some embodiments, the methods comprise increasing or decreasing the frequency of administration of ibrutinib. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

Described herein, in certain embodiments, are methods for optimizing the treatment of DLBCL in a patient in need thereof comprising: (a) determining an expression level of CCL3 and/or CCL4 in a sample from the patient following administration of a therapeutically effective amount of a BTK inhibitor; and (b) modifying, discontinuing, or continuing the treatment based on the expression of CCL3 and/or CCL4 relative to a control or reference level. In some embodiments, the methods comprise increasing or decreasing the dosage of the BTK inhibitor. In some embodiments, the methods comprise increasing or decreasing the frequency of administration of the BTK inhibitor. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the methods comprise an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation.

In some embodiments, the DLBCL is Activated B cell-like (ABC) subtype of DLBCL. In some embodiments, the ABC-DLBCL is characterized by a CD79B mutation. In some embodiments, the CD79B mutation is a mutation of the immunoreceptor tyrosine-based activation motif (ITAM) signaling module. In some embodiments, the CD79B mutation is a missense mutation of the first immunoreceptor tyrosine-based activation motif (ITAM) tyrosine. In some embodiments, the CD79B mutation increases surface BCR expression and attenuates Lyn kinase activity. In some embodiments, the ABC-DLBCL is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the ABC-DLBCL is characterized by a mutation in MyD88, A20, or a combination thereof. In some embodiments, the MyD88 mutation is the amino acid substitution L265P in the MYD88 Toll/IL-1 receptor (TIR) domain. In some embodiments, the methods further comprise administering an additional anticancer agent.

Described herein, in certain embodiments, are methods for treating DLBCL in a patient in need thereof comprising: (a) administering a treatment comprising a therapeutically effective amount of ibrutinib; (b) determining an expression level of CCL3 and/or CCL4 in a sample from the patient following administration of the treatment; and (c) continuing the treatment if the expression of CCL3 and/or CCL4 is decreased by a predetermined amount relative to the expression level of CCL3 and/or CCL4 prior to treatment. Also, described herein, in certain embodiments, are methods for treating DLBCL in a patient in need thereof comprising: (a) administering a treatment comprising a therapeutically effective amount of ibrutinib; (b) determining an expression level of CCL3 and/or CCL4 in a sample from the patient following administration of the treatment; and (c) discontinuing the treatment if the expression of CCL3 and/or CCL4 is not decreased by a predetermined amount relative to the expression level of CCL3 and/or CCL4 prior to treatment. In some embodiments, the predetermined amount is a decrease in the level of CCL3 and/or CCL4 expression by % 5%, 10%, 15%, 20%, 25%, 30%, 35%, 40%, 45%, 50%, 55%, 60%, 65%, 70%, 75%, 80%, 85%, 90%, 95% 99% or greater following treatment with ibrutinib. In some embodiments, the predetermined amount is a decrease in the level of CCL3 and/or CCL4 expression following treatment with ibrutinib to the level of expression of CCL3 and/or CCL4 in a human that does not have DLBCL. In some embodiments, the expression level of CCL3 and/or CCL4 is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with ibrutinib. In some embodiments, continuing the treatment comprises increasing or decreasing the dosage of ibrutinib. In some embodiments, continuing the treatment comprises increasing or decreasing the frequency of administration of ibrutinib. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, the sample is a serum sample. In some embodiments, measuring the amount of CCL3 and/or CCL4 proteins comprises detecting the CCL3 and/or CCL4 proteins with an antibody. In some embodiments, determining an expression level of CCL3 and/or CCL4 using an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods further comprise performing polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is Activated B cell-like (ABC) subtype of DLBCL. In some embodiments, the ABC-DLBCL is characterized by a CD79B mutation. In some embodiments, the CD79B mutation is a mutation of the immunoreceptor tyrosine-based activation motif (ITAM) signaling module. In some embodiments, the CD79B mutation is a missense mutation of the first immunoreceptor tyrosine-based activation motif (ITAM) tyrosine. In some embodiments, the CD79B mutation increases surface BCR expression and attenuates Lyn kinase activity. In some embodiments, the ABC-DLBCL is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the ABC-DLBCL is characterized by a mutation in MyD88, A20, or a combination thereof. In some embodiments, the MyD88 mutation is the amino acid substitution L265P in the MYD88 Toll/IL-1 receptor (TIR) domain. In some embodiments, the methods further comprise administering an additional anticancer agent.

Described herein, in certain embodiments, are methods for treating DLBCL in a patient in need thereof comprising: (a) administering a treatment comprising a therapeutically effective amount of a BTK inhibitor; (b) determining an expression level of CCL3 and/or CCL4 in a sample from the patient following administration of the treatment; and (c) continuing the treatment if the expression of CCL3 and/or CCL4 is decreased by a predetermined amount relative to the expression level of CCL3 and/or CCL4 prior to treatment. Also, described herein, in certain embodiments, are methods for treating DLBCL in a patient in need thereof comprising: (a) administering a treatment comprising a therapeutically effective amount of a BTK inhibitor; (b) determining an expression level of CCL3 and/or CCL4 in a sample from the patient following administration of the treatment; and (c) discontinuing the treatment if the expression of CCL3 and/or CCL4 is not decreased by a predetermined amount relative to the expression level of CCL3 and/or CCL4 prior to treatment. In some embodiments, the predetermined amount is a decrease in the level of CCL3 and/or CCL4 expression by % 5%, 10%, 15%, 20%, 25%, 30%, 35%, 40%, 45%, 50%, 55%, 60%, 65%, 70%, 75%, 80%, 85%, 90%, 95% 99% or greater following treatment with the BTK inhibitor. In some embodiments, the predetermined amount is a decrease in the level of CCL3 and/or CCL4 expression following treatment with the BTK inhibitor to the level of expression of CCL3 and/or CCL4 in a human that does not have DLBCL. In some embodiments, the expression level of CCL3 and/or CCL4 is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with the BTK inhibitor. In some embodiments, continuing the treatment comprises increasing or decreasing the dosage of the BTK inhibitor. In some embodiments, continuing the treatment comprises increasing or decreasing the frequency of administration of the BTK inhibitor. In some embodiments, the sample is a blood sample or a serum sample. In some embodiments, determining an expression level of CCL3 and/or CCL4 in the sample comprises measuring the amount of CCL3 and/or CCL4 proteins in the sample. In some embodiments, the sample is a serum sample. In some embodiments, measuring the amount of CCL3 and/or CCL4 proteins comprises detecting the CCL3 and/or CCL4 proteins with an antibody. In some embodiments, determining an expression level of CCL3 and/or CCL4 using an Enzyme-linked Immunosorbent Assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods further comprise performing polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is Activated B cell-like (ABC) subtype of DLBCL. In some embodiments, the ABC-DLBCL is characterized by a CD79B mutation. In some embodiments, the CD79B mutation is a mutation of the immunoreceptor tyrosine-based activation motif (ITAM) signaling module. In some embodiments, the CD79B mutation is a missense mutation of the first immunoreceptor tyrosine-based activation motif (ITAM) tyrosine. In some embodiments, the CD79B mutation increases surface BCR expression and attenuates Lyn kinase activity. In some embodiments, the ABC-DLBCL is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the ABC-DLBCL is characterized by a mutation in MyD88, A20, or a combination thereof. In some embodiments, the MyD88 mutation is the amino acid substitution L265P in the MYD88 Toll/IL-1 receptor (TIR) domain. In some embodiments, the methods further comprise administering an additional anticancer agent.

Described herein, in some embodiments, are methods for treating DLBCL in a patient in need thereof comprising determining a pre-administration expression level of CCL3 and/or CCL4 in a sample from the patient prior to administration of a BTK inhibitor; and administering to the patient a therapeutically effective amount of BTK inhibitor if the pre-administration expression of CCL3 and/or CCL4 is increased relative to a control or reference level. In some embodiments, the administering is of a dose of BTK inhibitor of about 140 mg to about 840 mg. In some embodiments, the dose is selected from the group consisting of 140 mg, 280 mg, 420 mg, 560 mg, 700 mg, or 840 mg. In some embodiments, the sample is a blood sample or a serum sample. In some embodiments, the determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the measuring the amount of CCL3 and/or CCL4 protein is with an enzyme-linked immunosorbent assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is activated B cell-like (ABC) subtype of DLBCL. In some embodiments, the ABC-DLBCL is characterized by a CD79B mutation or a CD79A mutation. In some embodiments, the ABC-DLBCL is characterized by a mutation in MyD88, A20, or a combination thereof. In some embodiments, the method further comprises determining a post-administration expression level of CCL3 and/or CCL4 in a sample from the patient following administration of the BTK inhibitor; and continuing treatment with BTK inhibitor if the post-administration expression of CCL3 and/or CCL4 is decreased by a predetermined amount relative to the pre-administration expression level of CCL3 and/or CCL4. In some embodiments, the treatment is with a once daily dose of BTK inhibitor of about 140 mg to about 840 mg. In some embodiments, the dose is selected from the group consisting of 140 mg, 280 mg, 420 mg, 560 mg, 700 mg, or 840 mg. In some embodiments, the predetermined amount is a decrease in the level of CCL3 and/or CCL4 expression by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of BTK inhibitor. In some embodiments, the predetermined amount is a decrease in the post-administration expression level of CCL3 and/or CCL4 to a level of expression of CCL3 and/or CCL4 in a human that does not have DLBCL.

Described herein, in some embodiments, are methods for treating DLBCL in a patient in need thereof comprising determining a pre-administration expression level of CCL3 and/or CCL4 in a sample from the patient prior to administration of ibrutinib; and administering to the patient a therapeutically effective amount of ibrutinib if the pre-administration expression of CCL3 and/or CCL4 is increased relative to a control or reference level. In some embodiments, the sample is a blood sample or a serum sample. In some embodiments, the determining an expression level of CCL3 and/or CCL4 in the sample comprises measuring the amount of CCL3 and/or CCL4 proteins in the sample. In some embodiments, measuring the amount of CCL3 and/or CCL4 proteins comprises detecting the CCL3 and/or CCL4 proteins with an antibody. In some embodiments, the measuring the amount of CCL3 and/or CCL4 protein is with an enzyme-linked immunosorbent assay (ELISA). In some embodiments, determining an expression level of CCL3 and/or CCL4 in the sample comprises measuring the amount of nucleic acid encoding CCL3 and/or CCL4 in the sample. In some embodiments, the sample comprises one or more tumor cells. In some embodiments, the nucleic acid is mRNA. In some embodiments, the methods further comprise amplification of the nucleic acid. In some embodiments, the methods comprise polymerase chain reaction. In some embodiments, the methods further comprise detection of the nucleic acid using a microarray. In some embodiments, the patient has received no previous anticancer therapy. In some embodiments, the anticancer therapy comprises chemotherapy or radiation. In some embodiments, the DLBCL is activated B cell-like (ABC) subtype of DLBCL. In some embodiments, the ABC-DLBCL is characterized by a CD79B mutation or a CD79A mutation. In some embodiments, the ABC-DLBCL is characterized by a mutation in MyD88, A20, or a combination thereof. In some embodiments, the method further comprises determining a post-administration expression level of CCL3 and/or CCL4 in a sample from the patient following administration of the ibrutinib; and continuing treatment with ibrutinib if the post-administration expression of CCL3 and/or CCL4 is decreased by a predetermined amount relative to the pre-administration expression level of CCL3 and/or CCL4. In some embodiments, the treatment is with a once daily dose of ibrutinib of about 140 mg to about 840 mg. In some embodiments, the dose is selected from the group consisting of 140 mg, 280 mg, 420 mg, 560 mg, 700 mg, or 840 mg. In some embodiments, the predetermined amount is a decrease in the level of CCL3 and/or CCL4 expression by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of ibrutinib. In some embodiments, the predetermined amount is a decrease in the post-administration expression level of CCL3 and/or CCL4 to a level of expression of CCL3 and/or CCL4 in a human that does not have DLBCL.

Described herein, in certain embodiments, are kits for carrying out any of the diagnostic and treatment methods provided herein. In some embodiments, the kits comprise one or more reagents for determining the expression level of CCL3 and/or CCL4 in a sample. In some embodiments, the kit comprises an antibody that binds to a CCL3 protein and/or an antibody that binds to a CCL4 protein. In some embodiments, the kit comprises a nucleic acid probe or primer that binds to nucleic acid encoding a CCL3 protein and/or a nucleic acid probe or primer that binds to a nucleic acid encoding a CCL4 protein.

### BRIEF DESCRIPTION OF THE DRAWINGS

FIG. 1 illustrates CCL3 secretion by DLBCL cell lines. The CCL3 level after anti-IgM (αIgM) stimulation and ibrutinib treatment was measured in four ABC subtype cell lines, TMD8 (FIG. 1A), HBL-1 (FIG. 1B), OCI-Ly10 (FIG. 1C) and OCI-Ly3 (FIG. 1D); and two GCB subtype cell lines, DB (FIG. 1E) and OCI-Ly19 (FIG. 1F) at baseline and after anti-IgM stimulation with or without ibrutinib treatment. The bar diagram represents the mean supernatant concentration of CCL3 from DLBCL cells cultured in complete medium (control), medium supplemented with 10 μg/mL of anti-IgM, 1 μM ibrutinib, or anti-IgM and ibrutinib. After treatment with ibrutinib, CCL3 level was significantly decreased in TMD8 and HBL-1 (wild type CARD11) cell lines (FIGS. 1A and 1B), but unaffected in OCI-Ly3 (CARD11 mutation, FIG. 1D) or the GCB cell lines DB and OCI-Ly19 (FIGS. 1E and 1F). In HBL-1, CCL3 level increased significantly from 1705.5 (±27.5) pg/mL to 10324.3 (±125.3) pg/mL after anti-IgM (αIgM) stimulation and decreased either to 313.2 (±6.8) pg/mL after ibrutinib treatment alone or to 2029.16 (±26.9) pg/mL after ibrutinib treatment in combination with anti-IgM (FIG. 1B). OCI-Ly10 also showed a significant decrease in CCL3 level after ibrutinib treatment; however, OCI-Ly10 secreted high concentration of CCL3 without stimulation, and there was no further increase in CCL3 concentration by stimulation with anti-IgM (FIG. 1C). The diagrams are representative of 4 independent experiments; **p<0.005, ***p<0.001.

FIG. 2 illustrates CCL4 secretion by DLBCL cell lines. The CCL4 level after anti-IgM (αIgM) stimulation and ibrutinib treatment was measured in four ABC subtype cell lines, TMD8 (FIG. 2A), HBL-1 (FIG. 2B), OCI-Ly10 (FIG. 2C) and OCI-Ly3 (FIG. 2D); and two GCB subtype cell lines, DB (FIG. 2E) and OCI-Ly19 (FIG. 2F). After treatment with ibrutinib, CCL4 level was significantly decreased in TMD8 and HBL-1 (wild type CARD11) cell lines (FIGS. 2A and 2B), but unaffected in OCI-Ly3 (CARD11 mutation, FIG. 2D) or the GCB cell lines DB and OCI-Ly19 (FIGS. 2E and 2F). Specifically, in both HBL-1 and TMD8 cell lines, CCL4 levels increased significantly after αIgM stimulation and decreased following either ibrutinib treatment alone or ibrutinib treatment in combination with αIgM stimulation (FIGS. 2A and 2B). OCI-Ly10 also showed a significant decrease in CCL4 level after ibrutinib treatment; however, OCI-Ly10 secreted high concentration of CCL4 without stimulation, and there was no further increase in CCL4 concentration by stimulation with anti-IgM (FIG. 2C).

FIG. 3 illustrates ibrutinib titration in HBL-1 cell line. Ibrutinib was either titrated in combination with 10 μg αIgM or alone (FIGS. 3B and 3D, red line). After BCR stimulation, treatment with ibrutinib at 500 nM concentration in combination with 10 μg αIgM resulted in the lowest CCL3 level (FIG. 3A-3B) and CCL4 level (FIG. 3C-3D). In FIGS. 3B and 3D, about a 2000 fold increase was observed for CCL3/4 levels after stimulation with 10 μg αIgM compared to ibrutinib treatment alone.

FIG. 4 illustrates ibrutinib titration in TMD8 cell line. Ibrutinib was either titrated in combination with 10 μg αIgM or alone (FIG. 4B-4D, red line). After BCR stimulation, CCL3 level was decreased at 1000 nM concentration of ibrutinib treatment in combination with 10 μg αIgM and this decreased level was maintained at 500 nM and 100 nM concentration of ibrutinib treatments (FIG. 4A-4B). Similarly, the CCL4 level was decreased at 1000 nM concentration of ibrutinib treatment in combination with 10 μg αIgM and this decreased level was maintained at 500 nM, 100 nM and 50 nM concentrations of ibrutinib treatments (FIG. 4C-4D).

FIG. 5 illustrates αIgM titration in HBL-1 and TMD8 cell lines. At 10 μg/mL concentration, both CCL3 and CCL4 levels were significantly increased in HBL-1 (FIG. 5A-5B) and TMD8 (FIG. 5C-5D) cell lines.

FIG. 6 shows plots of the distribution of serum levels of CCL3 (FIG. 6A) and CCL4 (FIG. 6B) in the MDA test cohort (N=102).

FIGS. 7A-7D show Kaplan-Meier curves for overall survival (OS) and progression free survival in the MDA test cohort (N=102) based on the serum levels of CCL3 and CCL4. Survival comparison between two groups was tested by log-rank test. OS difference between low (<40 pg/ml) and high (≧40 pg/ml) serum CCL3 level (FIG. 7A), PFS difference between low (<40 pg/ml) and high (≧40 pg/ml) serum CCL3 level (FIG. 7B), OS difference between low (<180 pg/ml) and high (≧180 pg/ml) serum CCL4 level (FIG. 7C), PFS difference between low (≧180 pg/ml) and high (≧180 pg/ml) serum CCL4 level (FIG. 7D).

FIG. 8 shows plots depicting serial quantification of serum CCL3 (FIG. 8A) and CCL4 (FIG. 8B) levels pre- and post-treatment in 19 patients with DLBCL.

FIGS. 9A-9B show Kaplan-Meier curves for OS difference between 4 groups based on both CCL3 and CCL4 levels (FIG. 9A), and PFS difference between 4 groups based on both CCL3 and CCL4 levels (FIG. 9B).

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information. Generally, the procedures for cell culture, cell infection, antibody production and molecular biology methods are methods commonly used in the art. Such standard techniques can be found, for example, in reference manual, such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error.

As used herein, “ACK” and “Accessible Cysteine Kinase” are synonyms. They mean a kinase with an accessible cysteine residue. ACKs include, but are not limited to, BTK, ITK, Bmx/ETK, TEC, EFGR, HER4, HER4, LCK, BLK, C-src, FGR, Fyn, HCK, Lyn, YES, ABL, Brk, CSK, FER, JAK3, SYK. In some embodiments, the ACK is a TEC family kinase. In some embodiments, the ACK is HER4. In some embodiments, the ACK is BTK. In some embodiments, the ACK is ITK.

As used herein, a Bruton's Tyrosine Kinase (BTK) polypeptide refers to any BTK protein or polypeptide, including, but not limited to, a recombinantly produced protein, a synthetically produced protein, a native BTK protein, and a BTK protein extracted from cells or tissues. A BTK polypeptide includes related polypeptides from different species including, but not limited to animals of human and non-human origin. BTK polypeptides of non-human origin include, but are not limited to, non-human primate (e.g. chimpanzee and ape), murine (e.g., mouse and rat), canine (dog), feline (cat), leporine (rabbit), avian (bird), bovine (cow), ovine (sheep), porcine (pig), equine (horse), piscine (fish), ranine (frog) and other mammalian or non-mammalian BTK polypeptides. Exemplary BTK polypeptides include, for example, orthologs from mouse (GenBank Accession No. AAB47246), dog (GenBank Accession No. XP_{—}549139. ), rat (GenBank Accession No. NP_{—}001007799), chicken (GenBank Accession No. NP_{—}989564), or zebra fish (GenBank Accession No. XP_{—}698117), and fusion proteins of any of the foregoing that exhibit kinase activity towards one or more substrates of Bruton's tyrosine kinase (e.g. a peptide substrate having the amino acid sequence “AVLESEEELYSSARQ” (SEQ ID NO: 5). A BTK polypeptide includes wild-type BTK, allelic variant isoforms, somatic mutations including those found in tumors or hematologic malignancies, synthetic molecules from nucleic acids, protein isolated from human tissue and cells, and modified forms thereof. The BTK polypeptides provided herein can be further modified by modification of the primary amino acid sequence, by deletion, addition, or substitution of one or more amino acids. A BTK polypeptide includes any BTK polypeptide or a portion thereof having BTK activity, such as kinase activity.

As used herein, a mutant BTK polypeptide, a mutant BTK protein, a modified BTK polypeptide, or a modified BTK protein or are used interchangeably herein and refer to a BTK polypeptide that is modified at one or more amino acid positions. Exemplary modifications include, but are not limited to, substitutions, deletions or additions of amino acids.

As used herein, the term “BTK inhibitor” or “BTK antagonist” refers to an agent that inhibits or reduces at least one activity of a BTK polypeptide. BTK activities include direct and indirect activities. Exemplary direct activities include, but are not limited to, association with a target molecule or phosphorylation of a target substrate (i.e., kinase activity). Exemplary indirect activities include, but are not limited to, activation or inhibition of a downstream biological event, such as for example activation of NF-κB-mediated gene transcription.

The term “irreversible inhibitor,” as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor.

The term “irreversible BTK inhibitor,” as used herein, refers to an inhibitor of BTK that can form a covalent bond with an amino acid residue of BTK. In one embodiment, the irreversible inhibitor of BTK can form a covalent bond with a cysteine residue of BTK; in particular embodiments, the irreversible inhibitor can form a covalent bond with a Cysteine 481 residue (or a homolog thereof) of BTK or a cysteine residue in the homologous corresponding position of another tyrosine kinase.

As used herein, inhibition of BTK activity refers any decrease in BTK activity in the presence of an inhibitor compared to the same activity in the absence of the inhibitor.

The terms “cancer” and “cancerous” refer to or describe the physiological condition in mammals that is typically characterized by unregulated cell growth. Examples of cancer include, but are not limited to, B-cell lymphoproliferative disorders (BCLDs), such as lymphoma and leukemia, and solid tumors. “B cell-related cancer” or “cancer of B-cell lineage” is intended to mean any type of cancer in which the dysregulated or unregulated cell growth is associated with B cells.

By “refractory” in the context of a cancer is intended to mean that the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

By “BTK-mediated signaling” it is intended to mean any of the biological activities that are dependent on, either directly or indirection, the activity of BTK. Examples of BTK-mediated signaling are signals that lead to proliferation and survival of BTK-expressing cells, and stimulation of one or more BTK-signaling pathways within BTK-expressing cells.

A BTK “signaling pathway” or “signal transduction pathway” is intended to mean at least one biochemical reaction, or a group of biochemical reactions, that results from the activity of BTK, and which generates a signal that, when transmitted through the signal pathway, leads to activation of one or more downstream molecules in the signaling cascade. Signal transduction pathways involve a number of signal transduction molecules that lead to transmission of a signal from the cell-surface across the plasma membrane of a cell, and through one or more in a series of signal transduction molecules, through the cytoplasm of the cell, and in some instances, into the cell's nucleus. Of particular interest to the present invention are BTK signal transduction pathways which ultimately regulate (either enhance or inhibit) the activation of NF-κB via the NF-κB signaling pathway.

As used herein, the terms “treat,” “treating” or “treatment,” and other grammatical equivalents, include alleviating, abating or ameliorating one or more symptoms of a disease or condition, ameliorating, preventing or reducing the appearance, severity or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. In a non-limiting example, for prophylactic benefit, a BTK inhibitor compound disclosed herein is administered to an individual at risk of developing a particular disorder, predisposed to developing a particular disorder, or to an individual reporting one or more of the physiological symptoms of a disorder. In some embodiments, a BTK inhibitor compound disclosed herein is administered to a subject following treatment with one or more therapeutic agents. In some embodiments, a BTK inhibitor compound disclosed herein is administered to a subject in combination with treatment with one or more therapeutic agents.

As used herein, prevention or prophylaxis refers to the reduction in the risk of developing a disease or condition.

The terms “co-administration” or “combination therapy” and the like, as used herein, are meant to encompass administration of the selected therapeutic agents to a single patient, and are intended to include treatment regimens in which the agents are administered by the same or different route of administration or at the same or different time.

The terms “effective amount”, “therapeutically effective amount” or “pharmaceutically effective amount” as used herein, refer to an amount of a therapeutic compound (e.g., a BTK inhibitor compound) that is sufficient to treat a disorder. In some embodiments, the result is a reduction in and/or alleviation of the signs, symptoms, or causes of a disorder, or any other desired alteration of a biological system. For example, an “effective amount” of a BTK inhibitor compound for therapeutic uses is the amount of the composition comprising a BTK inhibitor compound disclosed herein required to provide a clinically significant decrease in a disorder. An appropriate “effective” amount in any individual case is determined using any suitable technique, (e.g., a dose escalation study).

The term “pharmaceutically acceptable” as used herein, refers to a material, (e.g., a carrier or diluent), which does not abrogate the biological activity or properties of a therapeutic compound (e.g., a BTK inhibitor compound) described herein, and is relatively nontoxic (i.e., the material is administered to an individual without causing undesirable biological effects or interacting in a deleterious manner with any of the components of the composition in which it is contained).

As used herein, a control refers to a sample that is substantially identical to the test sample, except that it is not treated with a test parameter, or, if it is a plasma sample, it can be from a normal patient not affected with the condition of interest. A control also can be an internal control.

As used herein, the terms “subject”, “individual” and “patient” are used interchangeably. None of the terms are to be interpreted as requiring the supervision of a medical professional (e.g., a doctor, nurse, physician's assistant, orderly, hospice worker). As used herein, the subject can be any animal, including mammals (e.g., a human or non-human animal) and non-mammals. In one embodiment of the methods and compositions provided herein, the mammal is a human.

### Overview:

Diffuse large B cell lymphoma (DLBCL) is the most prevalent type of aggressive non-Hodgkin's lymphoma (NHL) in the United States. Clinical courses of patients with DLBCL are highly heterogeneous. While majority of the patients with DLBCL show response to the initial treatment, approximately one-third of patients have refractory disease or experience relapse after the standard therapies. DLBCL is a clinically and biologically heterogeneous disease, which can be demonstrated by several clinical and molecularly defined prognostic models. In certain instances, gene expression profiling (GEP) has been employed for dissecting the molecular heterogeneity and for predicting outcome in DLBCL. GEP can distinguish two prognostic subtypes, germinal center B cell-like (GCB) and activated B cell-like (ABC) DLBCL, among whose functional differences include activity of B cell receptor (BCR) signaling. ABC DLBCL cells have chronic active BCR signaling, upon which their survival is highly dependent.

B cell receptor (BCR) signaling is a critical growth and survival pathway in various B cell malignancies, including DLBCL. Upon BCR stimulation, normal and malignant B cells secrete the chemokines CCL3 and CCL4 (MIP-1 α and β) which promotes B cell interactions with accessory cells, such as T helper cells. CCL3 and CCL4 are chemokines of the CC subfamily and inducible in a number of hematopoietic cells, particularly those involved in adaptive immune responses (macrophages, dendritic cells, and B and T lymphocytes). CCL3 signals through the chemokine receptors CCR1 and CCR5, whereas CCL4 signals only through CCR5. CCL3 is a key response gene in B cells, which is up-regulated by BCR signaling, and repressed by Bcl-6. Plasma CCL3 and CCL4 levels are elevated in patients with B-cell malignancies, such as DLBCL and chronic lymphocytic leukemia (CLL). In DLBCL, SCYA3, the gene encoding CCL3, was highly expressed in the ABC subtype in DLBCL.

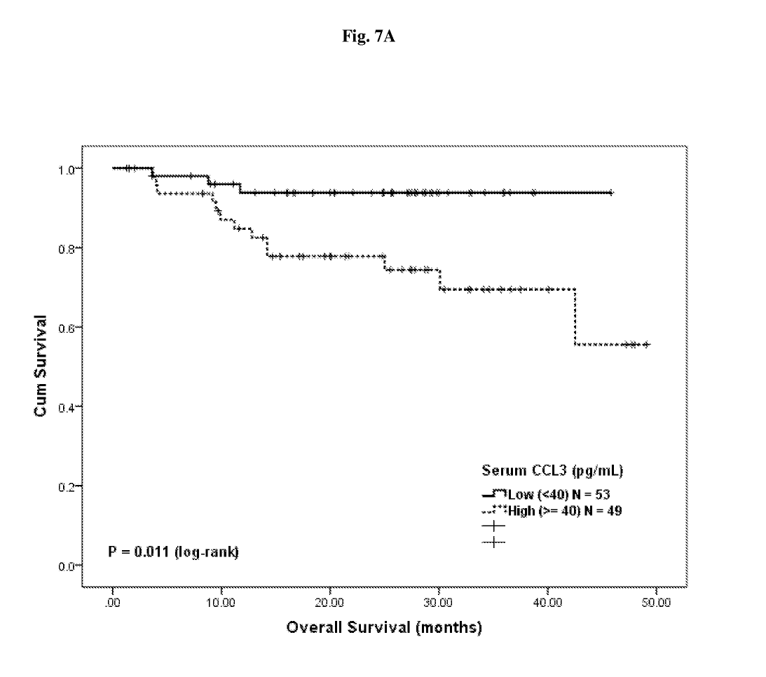

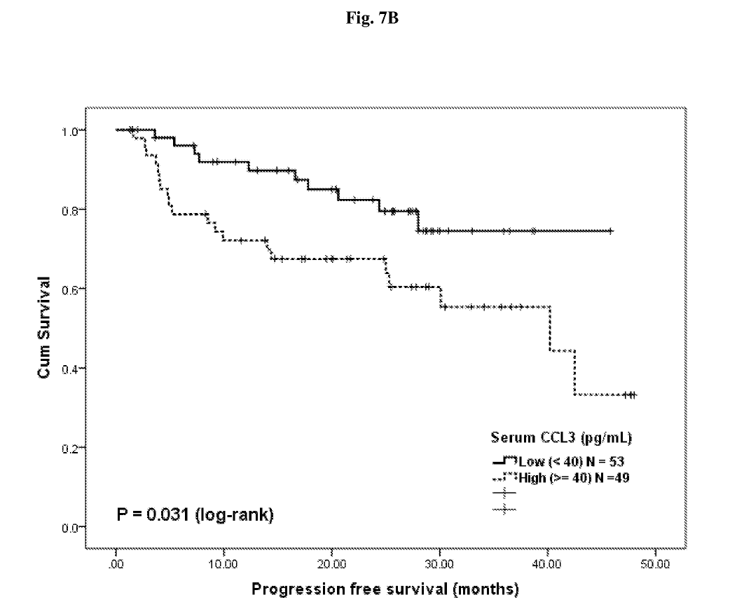

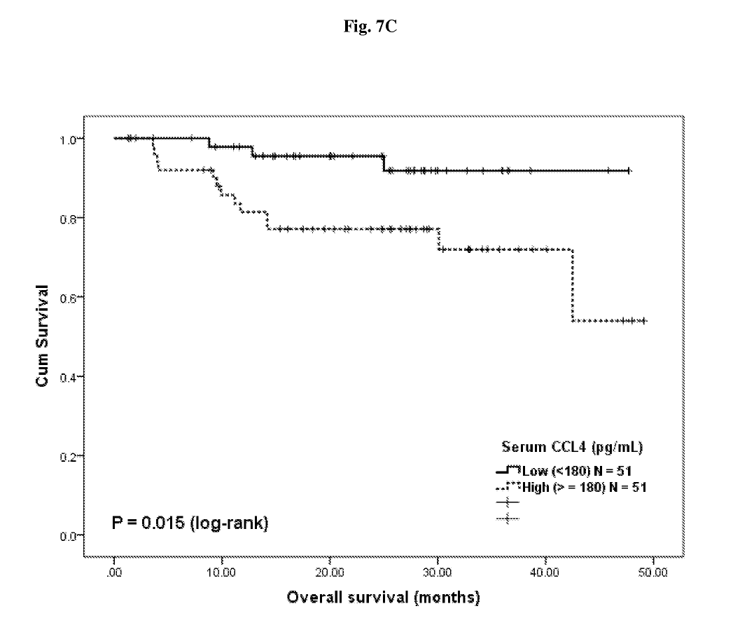

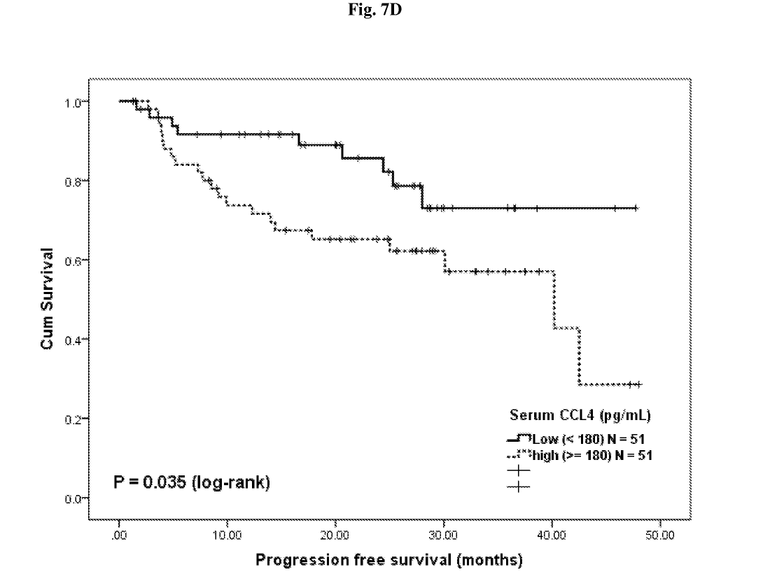

As demonstrated herein, CCL3 and CCL4 protein concentrations can be employed as biomarkers of BCR activation and as prognostic markers in DLBCL. The Examples provided demonstrate that high serum CCL3 level correlates with poor prognostic features in DLBCL and is associated with worse outcome in DLBCL (FIG. 7). For example, ABC DLBCL cells, but not GCB cells, secrete high levels of CCL3 and CCL4 after BCR triggering, which was sensitive to inhibition with the BTK inhibitor ibrutinib, as was baseline secretion of these chemokines, even at low nanomolar drug concentrations. It was also found that high CCL3 (≧40 pg/ml) serum concentrations correlated with higher international prognostic index (IPI), LDH, and β2 microglobulin, as did CCL4 (≧180 pg/ml) with advanced Ann Arbor stages. High CCL3 correlated with significantly shorter progression-free and overall survival. Further, a correlation between anti-IgM responsiveness in the ABC DLBL subtype in comparison with the GCB subtype, as well as sensitivity to ibrutinib treatment was observed. Furthermore, in most patients, high concentrations of serum CCL3 and CCL4 returned to low levels after ibrutinib therapy. One patient in whom CCL3 levels did not return to low levels later experienced relapse of his disease. These findings establish CCL3 and CCL4 protein concentrations as biomarkers for BCR pathway activation and prognosis in DLBCL and assessment of efficacy of BTK inhibitor therapies.

Early and effective treatment of DLBCL is a critical factor affecting the survival of DLBCL patients. The selection of treatment regimens against which DLBCL is resistant delays the onset of effective treatment of the cancer and can lead to growth and spread of the cancer. This, in turn, can have a negative effect on the patient's treatment outcome. Tumor-specific characteristics that are associated with responsiveness to an anti-cancer agent, e.g., a BTK inhibitor, such as the expression of one or more specific genes and/or encoded proteins are useful as a prognostic biomarker for identifying potential patients likely to respond or fail treatment with an BTK inhibitor at an earlier stage. As a result, patients suffering from DLBCL expressing such a biomarker can be selected for treatment with a BTK inhibitor. In addition, the biomarker can be employed for assessing the response to treatment with a BTK inhibitor.

The methods provided herein relate to the use of CCL3 and/or CCL4 (MIP-1α and β) expression as a predictive biomarker for identifying responder populations, especially those patients that are likely to be sensitive to treatment with a BTK inhibitor. The methods provided herein provide clinical advantages to the diagnosis and treatment of DLBCL, including easy access to samples, given that CCL3/CCL4 can reliably be quantified in plasma and serum samples, low costs of analyses, and rapid modulation (normalization within days) by therapies targeting the BCR. In some embodiments, serum levels of CCL3 can be easily quantified by Enzyme-Linked Immunosorbent Assay (ELISA) or other rapid protein detection methods.

The methods described herein are supported by the examples provided herein that show that increased expression levels of CCL3 and/or CCL4 prior to treatment with a BTK inhibitor is predictive of a positive treatment outcome with a BTK inhibitor (e.g., ibrutinib) in patients having DLBCL. The data demonstrate that DLBCL tumor cells exhibiting increased expression levels of CCL3 and/or CCL4 relative to normal cells are sensitive to treatment with a BTK inhibitor (e.g., ibrutinib). Conversely, DLBCL tumor cells that do not exhibit increased expression levels of CCL3 and/or CCL4 relative to normal cells are resistant to treatment with a BTK inhibitor (e.g., ibrutinib). Accordingly, in some embodiments, patients presenting with higher than normal expression levels of CCL3 and/or CCL4 are likely to be sensitive to treatment with a BTK inhibitor (e.g., ibrutinib). In some embodiments, patients exhibiting about the same as or lower levels of CCL3 and/or CCL4 expression relative to normal are likely to be resistant to treatment with a BTK inhibitor. Thus, measurement of CCL3 and/or CCL4 expression level, gene or protein expression, is particularly useful to identify patients likely to respond to therapy with a BTK inhibitor (e.g., ibrutinib). In some embodiments, the BTK inhibitor is an irreversible BTK inhibitor. In some embodiments, the inhibitor is ibrutinib.

In addition, the examples provided herein show that DLBCL patients with increased expression levels of CCL3 and/or CCL4 prior to treatment with a BTK inhibitor, exhibit rapid decrease and normalization of CCL3 and/or CCL4 expression following treatment with a BTK inhibitor (e.g., ibrutinib). The degree to which the CCL3 and/or CCL4 expression decreases in response to treatment with a BTK inhibitor is predictive of a positive treatment outcome with a BTK inhibitor (e.g., ibrutinib). Accordingly, provided herein are methods of predicting a positive response of a patient having DLBCL to a BTK inhibitor by assessing the degree to which CCL3 and/or CCL4 expression decreases following treatment with the BTK inhibitor. In some embodiments, the BTK inhibitor is an irreversible BTK inhibitor. In some embodiments, the inhibitor is ibrutinib.

Also provided are methods and procedures for determining patient sensitivity to a BTK inhibitor. Also provided are methods for determining or predicting whether an individual requiring therapy for DLBCL will or will not respond to treatment, prior to administration of the treatment, wherein the treatment comprises administration of one or more BTK inhibitors.

It is noted that while the examples demonstrate use of the irreversible BTK inhibitor ibrutinib, the results would apply equally across to other BTK inhibitors, including reversible and other irreversible BTK inhibitors. Such inhibitors are known in the art and include, for example, ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, PRN1008 (Principia), CTP-730 (Concert Pharmaceuticals), and GDC-0853 (Genentech).

### Compounds

Disclosed herein, in certain embodiments, are methods for selecting patients diagnosed with diffuse large B-cell lymphoma (DLBCL) for treatment with an inhibitor of Bruton's tyrosine kinase (BTK) based on the level of expression the biomarkers CCL3 and/or CCL4. In some embodiments, the methods comprise identifying patient likely to respond to treatment with a BTK inhibitor. In some embodiments, the methods comprise determining a treatment regimen. Also disclosed herein, in certain embodiments, are methods for evaluating treatment of diffuse large B-cell lymphoma (DLBCL), in an individual in need thereof, comprising: administering to the individual a therapeutically effective amount of an inhibitor of a BTK inhibitor and determining the responsiveness of the patient to treatment based on the level of expression of CCL3 and/or CCL4. Also disclosed herein, in certain embodiments, are methods for treatment of diffuse large B-cell lymphoma (DLBCL), in an individual in need thereof, comprising: administering to the individual a therapeutically effective amount of an inhibitor of a BTK inhibitor, determining the responsiveness of the patient to treatment based on the level of expression of CCL3 and/or CCL4, and continuing treatment if the level of CCL3 and/or CCL4 expression has decreased by a predetermined amount. Also disclosed herein, in certain embodiments, are methods for treatment of diffuse large B-cell lymphoma (DLBCL), in an individual in need thereof, comprising: administering to the individual a therapeutically effective amount of an inhibitor of a BTK inhibitor, determining the responsiveness of the patient to treatment based on the level of expression of CCL3 and/or CCL4, and discontinuing treatment if the level of CCL3 and/or CCL4 expression has not decreased by a predetermined amount. As demonstrated herein, high levels of expression of CCL3 and/or CCL4 prior to treatment are predictive of a therapeutic response to treatment with a BTK inhibitor (e.g., ibrutinib). In addition, a decrease in levels of CCL3 and/or CCL4 expression (e.g., normalization of expression) following administration with a BTK inhibitor (e.g., ibrutinib) is predictive of the efficacy of the BTK inhibitor for treatment of DLBCL.

In the following description of irreversible BTK compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. In addition, nucleic acid and amino acid sequences for BTK (e.g., human BTK) are known in the art as disclosed in, e.g., U.S. Pat. No. 6,326,469. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

The BTK inhibitor compounds described herein are selective for BTK and kinases having a cysteine residue in an amino acid sequence position of the tyrosine kinase that is homologous to the amino acid sequence position of cysteine 481 in BTK. Generally, an irreversible inhibitor compound of BTK used in the methods described herein is identified or characterized in an in vitro assay, e.g., an acellular biochemical assay or a cellular functional assay. Such assays are useful to determine an in vitro IC₅₀ for an irreversible BTK inhibitor compound.

For example, an acellular kinase assay can be used to determine BTK activity after incubation of the kinase in the absence or presence of a range of concentrations of a candidate irreversible BTK inhibitor compound. If the candidate compound is in fact an irreversible BTK inhibitor, BTK kinase activity will not be recovered by repeat washing with inhibitor-free medium. See, e.g., J. B. Smaill, et al. (1999), J. Med. Chem, 42(10):1803-1815. Further, covalent complex formation between BTK and a candidate irreversible BTK inhibitor is a useful indicator of irreversible inhibition of BTK that can be readily determined by a number of methods known in the art (e.g., mass spectrometry). For example, some irreversible BTK-inhibitor compounds can form a covalent bond with Cys 481 of BTK (e.g., via a Michael reaction).

Cellular functional assays for BTK inhibition include measuring one or more cellular endpoints in response to stimulating a BTK-mediated pathway in a cell line (e.g., BCR activation in Ramos cells) in the absence or presence of a range of concentrations of a candidate irreversible BTK inhibitor compound. Useful endpoints for determining a response to BCR activation include, e.g., autophosphorylation of BTK, phosphorylation of a BTK target protein (e.g., PLC-γ), and cytoplasmic calcium flux.

High-throughput assays for many acellular biochemical assays (e.g., kinase assays) and cellular functional assays (e.g., calcium flux) are well known to those of ordinary skill in the art. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification and characterization of a large number of irreversible BTK compounds without undue effort.

In some embodiments, the BTK inhibitor is selected from the group consisting of a small organic molecule, a macromolecule, a peptide or a non-peptide.

In some embodiments, the BTK inhibitor provided herein is a reversible or irreversible inhibitor. In certain embodiments, the BTK inhibitor is an irreversible inhibitor.

In some embodiments, the irreversible BTK inhibitor forms a covalent bond with a cysteine sidechain of a Bruton's tyrosine kinase, a Bruton's tyrosine kinase homolog, or a BTK tyrosine kinase cysteine homolog.

Irreversible BTK inhibitor compounds can be used for the manufacture of a medicament for treating any of the foregoing conditions (e.g., autoimmune diseases, inflammatory diseases, allergy disorders, B-cell proliferative disorders, or thromboembolic disorders).

In some embodiments, the irreversible BTK inhibitor compound used for the methods described herein inhibits BTK or a BTK homolog kinase activity with an in vitro IC₅₀ of less than 10 μM (e.g., less than less than 0.5 μM, less than 0.4 μM, less than 0.3 μM, less than 0.1, less than 0.08 μM, less than 0.06 μM, less than 0.05 μM, less than 0.04 μM, less than 0.03 μM, less than less than 0.02 μM, less than 0.01, less than 0.008 μM, less than 0.006 μM, less than 0.005 μM, less than 0.004 μM, less than 0.003 μM, less than less than 0.002 μM, less than 0.001, less than 0.00099 μM, less than 0.00098 μM, less than 0.00097 μM, less than 0.00096 μM, less than 0.00095 μM, less than 0.00094 μM, less than 0.00093 μM, less than 0.00092, or less than 0.00090 μM).

In some embodiments, the irreversible BTK inhibitor compound is selected from among ibrutinib (PCI-32765), PCI-45292, PCI-45466, AVL-101, AVL-291, AVL-292, or ONO-WG-37. In some embodiments, the irreversible BTK inhibitor compound is ibrutinib.

In one embodiment, the irreversible BTK inhibitor compound selectively and irreversibly inhibits an activated form of its target tyrosine kinase (e.g., a phosphorylated form of the tyrosine kinase). For example, activated BTK is transphosphorylated at tyrosine 551. Thus, in these embodiments the irreversible BTK inhibitor inhibits the target kinase in cells only once the target kinase is activated by the signaling events.

In other embodiments, the BTK inhibitor used in the methods describe herein has the structure of any of Formula (A). Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

Definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques are optionally used for chemical syntheses, chemical analyses, pharmaceutical preparation, formulation, and delivery, and treatment of patients. Standard techniques are optionally used for recombinant DNA, oligonucleotide synthesis, and tissue culture and transformation (e.g., electroporation, lipofection). Reactions and purification techniques are performed using documented methodologies or as described herein.

It is to be understood that the methods and compositions described herein are not limited to the particular methodology, protocols, cell lines, constructs, and reagents described herein and as such optionally vary. It is also to be understood that the terminology used herein is for the purpose of describing particular embodiments only, and is not intended to limit the scope of the methods and compositions described herein, which will be limited only by the appended claims.

Unless stated otherwise, the terms used for complex moieties (i.e., multiple chains of moieties) are to be read equivalently either from left to right or right to left. For example, the group alkylenecycloalkylene refers both to an alkylene group followed by a cycloalkylene group or as a cycloalkylene group followed by an alkylene group.

The suffix “ene” appended to a group indicates that such a group is a diradical. By way of example only, a methylene is a diradical of a methyl group, that is, it is a —CH₂— group; and an ethylene is a diradical of an ethyl group, i.e., —CH₂CH₂—.

An “alkyl” group refers to an aliphatic hydrocarbon group. The alkyl moiety includes a “saturated alkyl” group, which means that it does not contain any alkene or alkyne moieties. The alkyl moiety also includes an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, includes branched, straight chain, or cyclic moieties. Depending on the structure, an alkyl group includes a monoradical or a diradical (i.e., an alkylene group), and if a “lower alkyl” having 1 to 6 carbon atoms.

As used herein, C₁-Cₓ includes C₁-C₂, C₁-C₃ . . . C₁-Cₓ.

The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which are either the same or different. The alkenyl moiety is optionally branched, straight chain, or cyclic (in which case, it is also known as a “cycloalkenyl” group). Depending on the structure, an alkenyl group includes a monoradical or a diradical (i.e., an alkenylene group). Alkenyl groups are optionally substituted. Non-limiting examples of an alkenyl group include —CH═CH₂, —C(CH₃)═CH₂, —CH═CHCH₃, —C(CH₃)═CHCH₃. Alkenylene groups include, but are not limited to, —CH═CH—, —C(CH₃)═CH—, —CH═CHCH₂—, —CH═CHCH₂CH₂— and —C(CH₃)═CHCH₂—. Alkenyl groups optionally have 2 to 10 carbons, and if a “lower alkenyl” having 2 to 6 carbon atoms.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which is either the same or different. The “R” portion of the alkynyl moiety may be branched, straight chain, or cyclic. Depending on the structure, an alkynyl group includes a monoradical or a diradical (i.e., an alkynylene group). Alkynyl groups are optionally substituted. Non-limiting examples of an alkynyl group include, but are not limited to, —C≡CH, —C≡CCH₃, —C≡CCH₂CH₃, and —C≡C≡CH₂—. Alkynyl groups optionally have 2 to 10 carbons, and if a “lower alkynyl” having 2 to 6 carbon atoms.

An “alkoxy” group refers to a (alkyl)O— group, where alkyl is as defined herein.

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, optionally form a cyclic ring system.

“Alkylaminoalkyl” refers to an alkyl radical, as defined herein, substituted with an alkylamine, as defined herein.

“Hydroxyalkylaminoalkyl” refers to an alkyl radical, as defined herein, substituted with an alkylamine, and alkylhydroxy, as defined herein.

“Alkoxyalkylaminoalkyl” refers to an alkyl radical, as defined herein, substituted with an alkylamine and substituted with an alkylalkoxy, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). In some embodiments, an amide moiety forms a linkage between an amino acid or a peptide molecule and a compound described herein, thereby forming a prodrug. Any amine, or carboxyl side chain on the compounds described herein can be amidified. The procedures and specific groups to make such amides are found in sources such as Greene and Wuts, Protective Groups in Organic Synthesis, 3^{rd} Ed., John Wiley & Sons, New York, N.Y., 1999, which is incorporated herein by reference for this disclosure.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are found in sources such as Greene and Wuts, Protective Groups in Organic Synthesis, 3^{rd} Ed., John Wiley & Sons, New York, N.Y., 1999, which is incorporated herein by reference for this disclosure.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e. a heteroatom). Heterocycle includes heteroaryl and heterocycloalkyl. Carbocycles and heterocycles can be optionally substituted.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “carbonyl” as used herein refers to a group containing a moiety selected from the group consisting of —C(O)—, —S(O)—, —S(O)2-, and —C(S)—, including, but not limited to, groups containing a least one ketone group, and/or at least one aldehyde group, and/or at least one ester group, and/or at least one carboxylic acid group, and/or at least one thioester group. Such carbonyl groups include ketones, aldehydes, carboxylic acids, esters, and thioesters. In some embodiments, such groups are a part of linear, branched, or cyclic molecules. The term “cycloalkyl” refers to a monocyclic or polycyclic radical that contains only carbon and hydrogen, and is optionally saturated, partially unsaturated, or fully unsaturated. Cycloalkyl groups include groups having from 3 to 10 ring atoms. Illustrative examples of cycloalkyl groups include the following moieties:

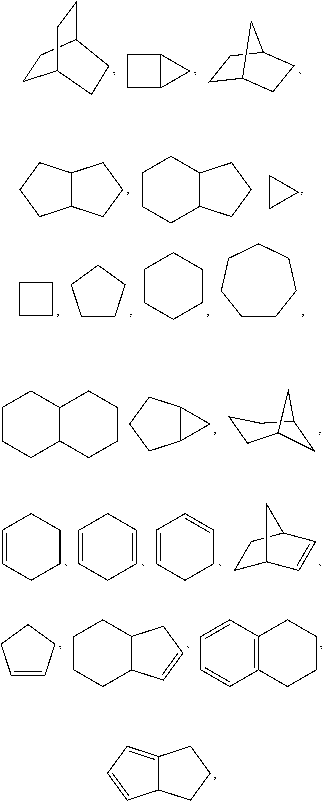

and the like. Depending on the structure, a cycloalkyl group is either a monoradical or a diradical (e.g., an cycloalkylene group), and if a “lower cycloalkyl” having 3 to 8 carbon atoms.

“Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, are optionally C-attached or N-attached where such is possible. For instance, a group derived from pyrrole includes pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The terms “heteroaryl” or, alternatively, “heteroaromatic” refers to an aromatic group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. Illustrative examples of heteroaryl groups include the following moieties:

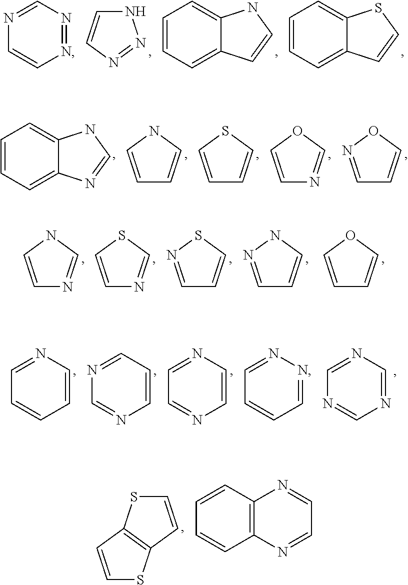

and the like. Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. In some embodiments, the radicals are fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

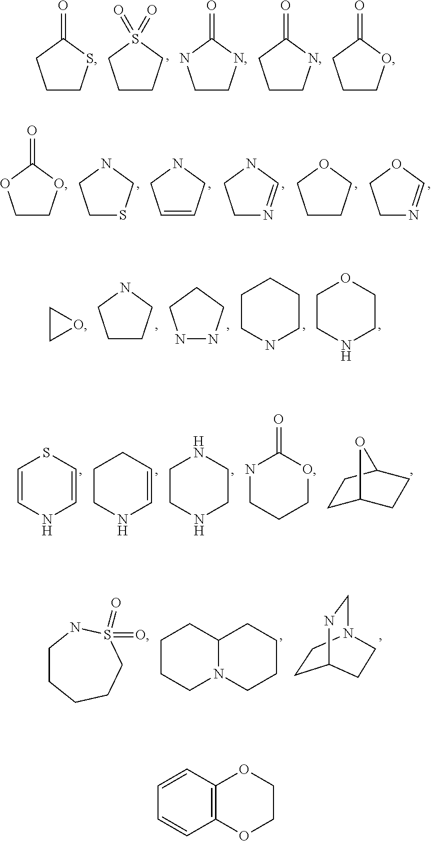

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

The term “halo” or, alternatively, “halogen” or “halide” means fluoro, chloro, bromo, and iodo.

The term “haloalkyl,” refers to alkyl structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

The term “fluoroalkyl,” as used herein, refers to alkyl group in which at least one hydrogen is replaced with a fluorine atom. Examples of fluoroalkyl groups include, but are not limited to, —CF₃, —CH₂CF₃, —CF₂CF₃, —CH₂CH₂CF₃ and the like.

As used herein, the term “heteroalkyl” refers to optionally substituted alkyl radicals in which one or more skeletal chain atoms is a heteroatom, e.g., oxygen, nitrogen, sulfur, silicon, phosphorus or combinations thereof. The heteroatom(s) are placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, in some embodiments, up to two heteroatoms are consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “SH” group is also referred to either as a thiol group or a sulfhydryl group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl. The protecting groups that form the protective derivatives of the above substituents include those found in sources such as Greene and Wuts, above.

### ACK Inhibitor Compounds

Disclosed herein, in certain embodiments, are methods for selecting patients diagnosed with diffuse large B-cell lymphoma (DLBCL) for treatment with an ACK inhibitor (e.g., an ITK or BTK inhibitor) based on the level of expression the biomarkers CCL3 and/or CCL4. In some embodiments, the methods comprise identifying patient likely to respond to treatment an ACK inhibitor (e.g., an ITK or BTK inhibitor). In some embodiments, the methods comprise determining a treatment regimen. Also disclosed herein, in certain embodiments, are methods for evaluating treatment of diffuse large B-cell lymphoma (DLBCL), in an individual in need thereof, comprising: administering to the individual a therapeutically effective amount of an inhibitor of an ACK inhibitor (e.g., an ITK or BTK inhibitor) and determining the responsiveness of the patient to treatment based on the level of expression of CCL3 and/or CCL4. Also disclosed herein, in certain embodiments, are methods for treatment of diffuse large B-cell lymphoma (DLBCL), in an individual in need thereof, comprising: administering to the individual a therapeutically effective amount of an inhibitor of an ACK inhibitor (e.g., an ITK or BTK inhibitor), determining the responsiveness of the patient to treatment based on the level of expression of CCL3 and/or CCL4, and continuing treatment if the level of CCL3 and/or CCL4 expression has decreased by a predetermined amount. Also disclosed herein, in certain embodiments, are methods for treatment of diffuse large B-cell lymphoma (DLBCL), in an individual in need thereof, comprising: administering to the individual a therapeutically effective amount of an inhibitor of an ACK inhibitor (e.g., an ITK or BTK inhibitor), determining the responsiveness of the patient to treatment based on the level of expression of CCL3 and/or CCL4, and discontinuing treatment if the level of CCL3 and/or CCL4 expression has not decreased by a predetermined amount. As demonstrated herein, high levels of expression of CCL3 and/or CCL4 prior to treatment are predictive of a therapeutic response to treatment with an ACK inhibitor (e.g., an ITK or BTK inhibitor). In addition, a decrease in levels of CCL3 and/or CCL4 expression (e.g., normalization of expression) following administration with an ACK inhibitor (e.g., an ITK or BTK inhibitor) is predictive of the efficacy of the ACK inhibitor for treatment of DLBCL.

The ACK inhibitor compounds described herein are selective for kinases having an accessible cysteine that is able to form a covalent bond with a Michael acceptor moiety on the inhibitor compound. In some embodiments, the cysteine residue is accessible or becomes accessible when the binding site moiety of the irreversible inhibitor binds to the kinase. That is, the binding site moiety of the irreversible inhibitor binds to an active site of the ACK and the Michael acceptor moiety of irreversible inhibitor gains access (in one embodiment the step of binding leads to a conformational change in the ACK, thus exposing the cysteine) or is otherwise exposed to the cysteine residue of the ACK; as a result a covalent bond is formed between the “S” of the cysteine residue and the Michael acceptor of the irreversible inhibitor. Consequently, the binding site moiety of the irreversible inhibitor remains bound or otherwise blocks the active site of the ACK.

In some embodiments, the ACK is BTK, a homolog of BTK or a tyrosine kinase having a cysteine residue in an amino acid sequence position that is homologous to the amino acid sequence position of cysteine 481 in BTK. In some embodiments, the ACK is ITK. In some embodiments, the ACK is HER4. Inhibitor compounds described herein include a Michael acceptor moiety, a binding site moiety and a linker that links the binding site moiety and the Michael acceptor moiety (and in some embodiments, the structure of the linker provides a conformation, or otherwise directs the Michael acceptor moiety, so as to improve the selectivity of the irreversible inhibitor for a particular ACK). In some embodiments, the ACK inhibitor inhibits ITK and BTK.

In some embodiments, the ACK inhibitor is a compound of Formula (A):

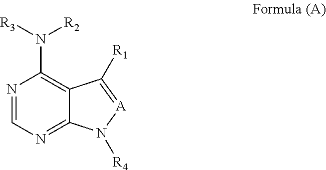

wherein
A is independently selected from N or CR₅;
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkyl), or -(substituted or unsubstituted C₂-C₆ alkenyl);
R₂ and R₃ are independently selected from H, lower alkyl and substituted lower alkyl;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—; L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is

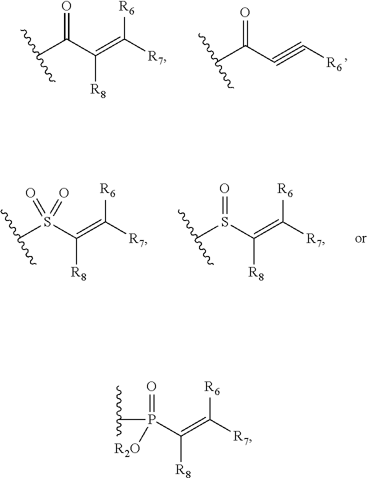

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₅ is H, halogen, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, O, S, —S(═O), S(═O)₂, NH, C(O), —NHC(O)O, —OC(O)NH, —NHC(O), or —C(O)NH;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
each R₁₁ is independently selected from H or alkyl; and pharmaceutically active metabolites, pharmaceutically acceptable solvates, pharmaceutically acceptable salts, or pharmaceutically acceptable prodrugs thereof.

In some embodiments, the compound of Formula (A) is a BTK inhibitor. In some embodiments, the compound of Formula (A) is an ITK inhibitor. In some embodiments, the compound of Formula (A) inhibits ITK and BTK. In some embodiments, the compound of Formula (A) has the structure:

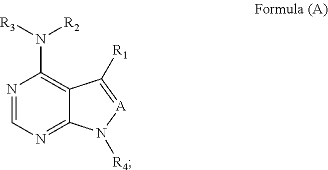

wherein:

A is N;

R₂ and R₃ are each H;

R₁ is phenyl-O-phenyl or phenyl-S-phenyl; and

R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroaryl, aryl, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle; or L₃, X and L₄ taken together form a nitrogen containing heterocyclic ring;
G is

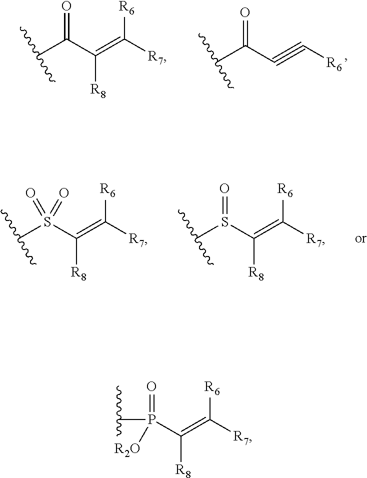

wherein,

R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl.

In some embodiments, the ACK inhibitor is (R)-1-(3-(4-amino-3-(4-phenoxyphenyl)-1H-pyrazolo[3,4-d]pyrimidin-1-yl)piperidin-1-yl)prop-2-en-1-one (i.e. PCI-32765/ibrutinib)

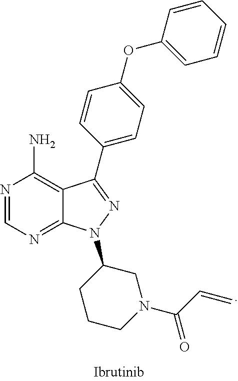 [SMILES: C=CC(=O)N1CCC[C@@H](n2nc(-c3ccc(Oc4ccccc4)cc3)c3c(N)ncnc32)C1]

In some embodiments, the ACK inhibitor is ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, the ACK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066).

In some embodiments, the ACK inhibitor is:

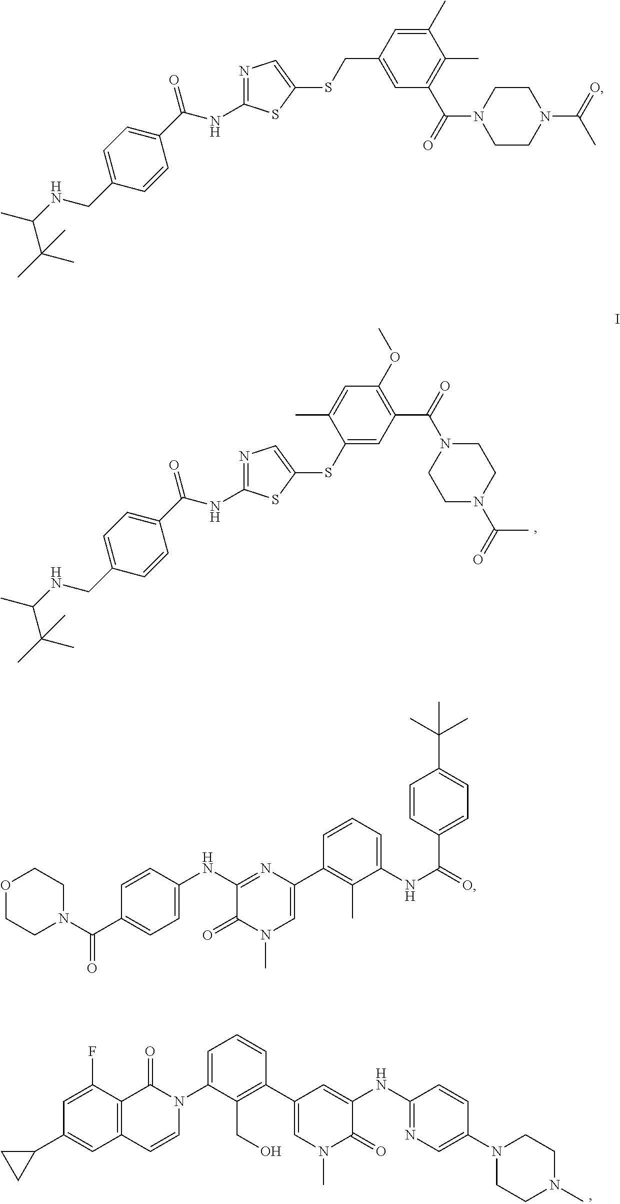

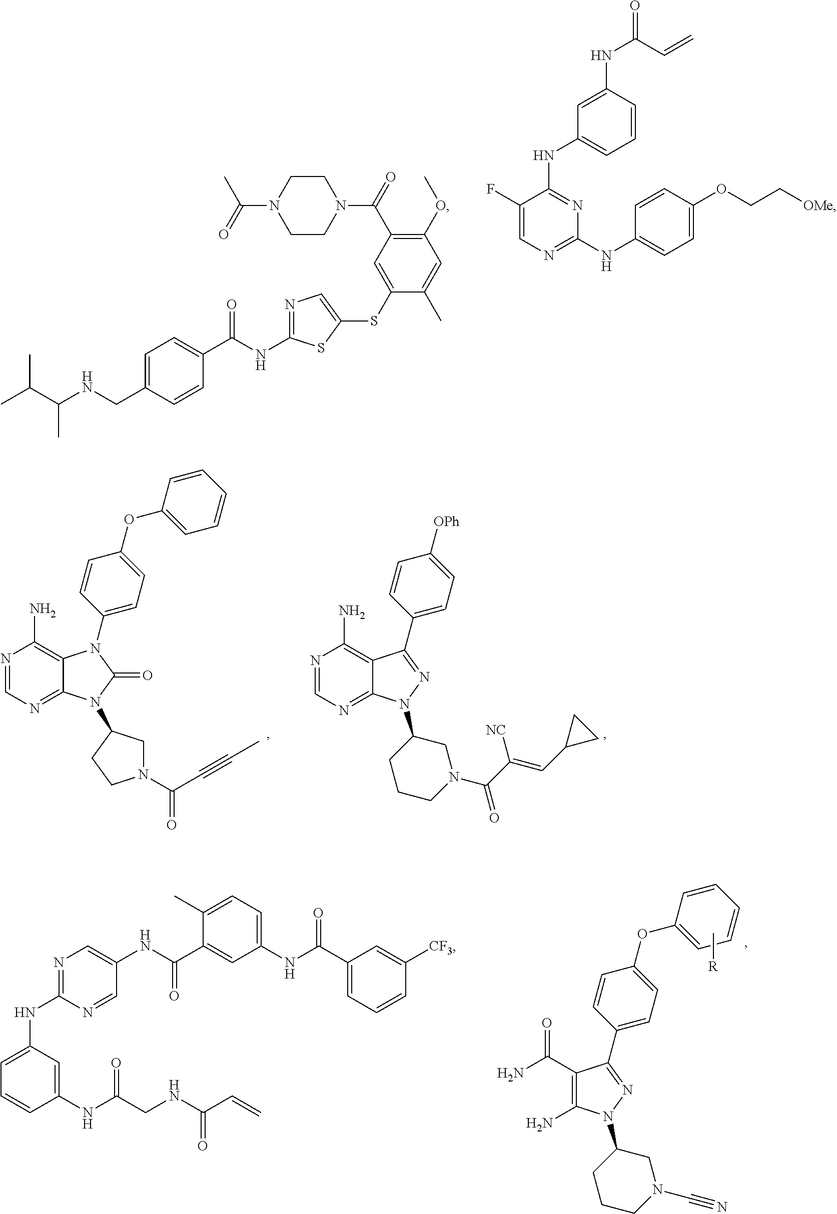

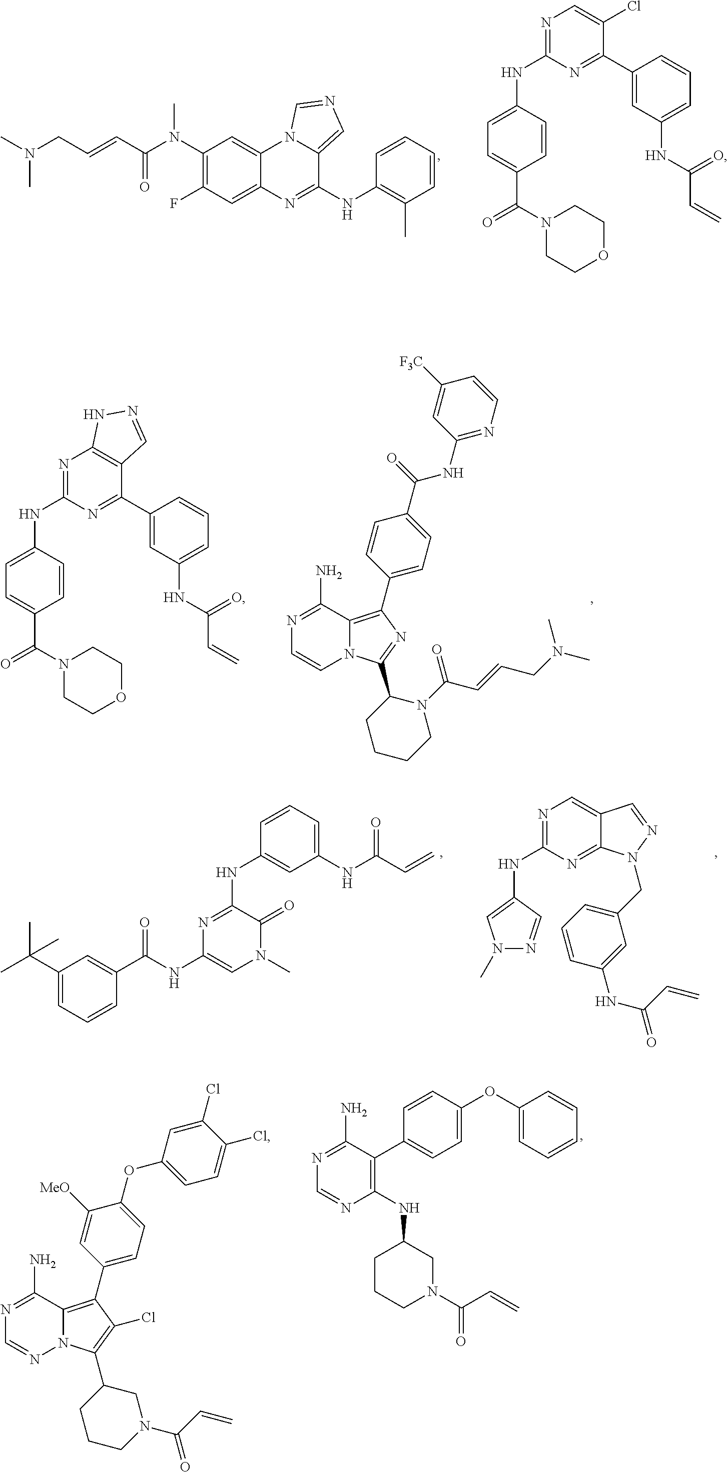

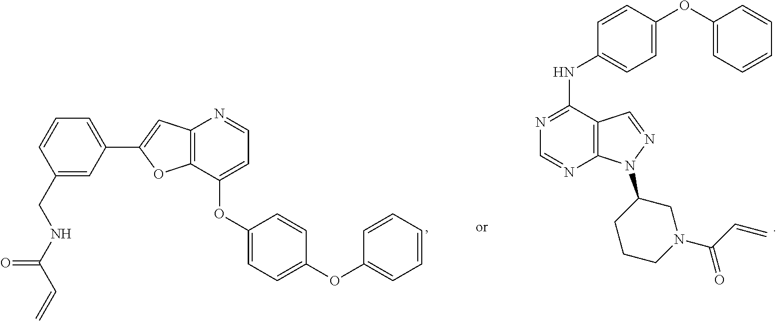 [SMILES: C=CC(=O)NCc1cccc(-c2cc3nccc(Oc4ccc(Oc5ccccc5)cc4)c3o2)c1 ; C=CC(=O)N1CCC[C@@H](n2ncc3c(Nc4ccc(Oc5ccccc5)cc4)ncnc32)C1]

### BTK Inhibitors

In some embodiments, the ACK inhibitor is a BTK inhibitor. The BTK inhibitor compounds described herein are selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction).

In some embodiments, the BTK inhibitor is a compound of Formula (A) having the structure:

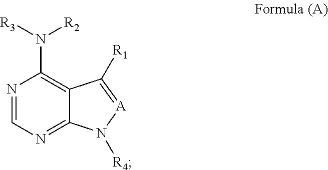

wherein:

### A is N;

R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═-NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;

### G is

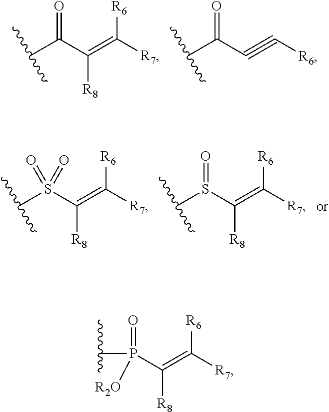

wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

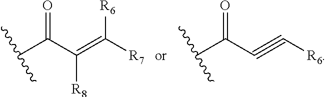

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

In some embodiments, the BTK inhibitor compound of Formula (A) has the following structure of Formula (B):

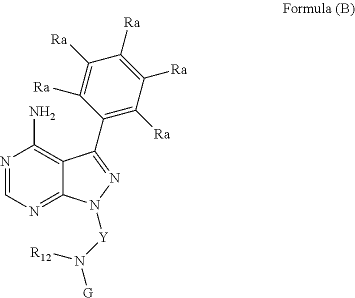

wherein:
Y is alkyl or substituted alkyl, or a 4-, 5-, or 6-membered cycloalkyl ring;
each Rₐ is independently H, halogen, —CF₃, —CN, —NO₂, OH, NH₂, -Lₐ-(substituted or unsubstituted alkyl), -Lₐ-(substituted or unsubstituted alkenyl), -Lₐ-(substituted or unsubstituted heteroaryl), or -Lₐ-(substituted or unsubstituted aryl), wherein Lₐ is a bond, O, S, —S(═O), —S(═O)₂, NH, C(O), CH₂, —NHC(O)O, —NHC(O), or —C(O)NH;

### G is

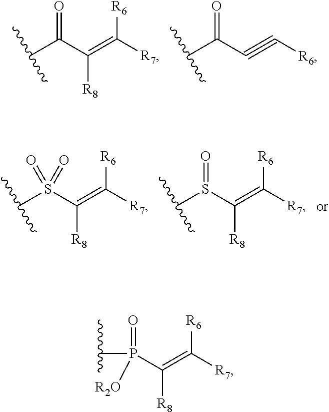

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₁₂ is H or lower alkyl; or
Y and R₁₂ taken together form a 4-, 5-, or 6-membered heterocyclic ring; and pharmaceutically acceptable active metabolites, pharmaceutically acceptable solvates, pharmaceutically acceptable salts, or pharmaceutically acceptable prodrugs thereof.

In some embodiments, G is selected from among

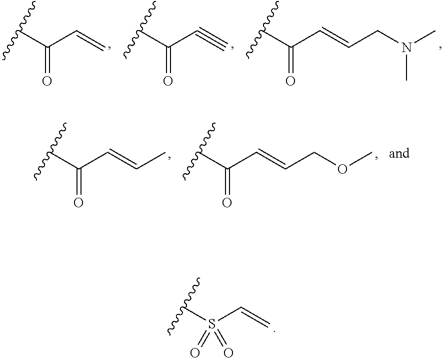

In some embodiments,

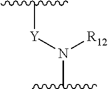

is selected from among

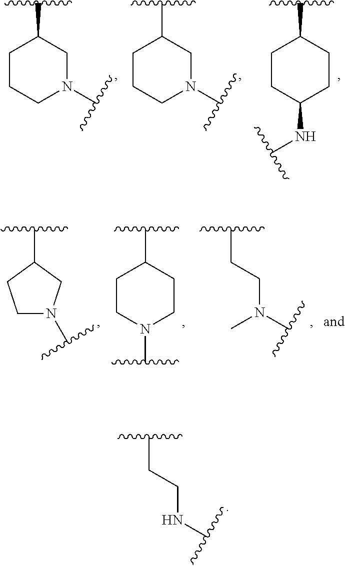

In some embodiments, the BTK inhibitor compound of Formula (B) has the following structure of Formula (C):

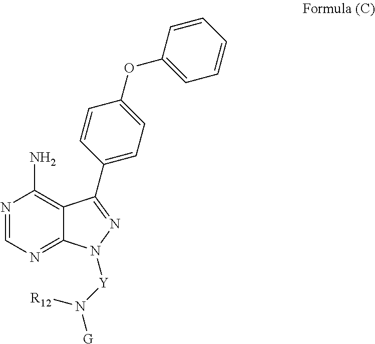

Y is alkyl or substituted alkyl, or a 4-, 5-, or 6-membered cycloalkyl ring;
R₁₂ is H or lower alkyl; or
Y and R₁₂ taken together form a 4-, 5-, or 6-membered heterocyclic ring;

### G is

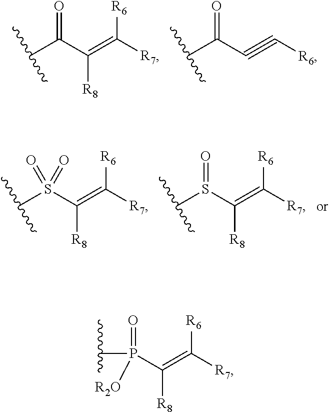

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl; and pharmaceutically acceptable active metabolites, pharmaceutically acceptable solvates, pharmaceutically acceptable salts, or pharmaceutically acceptable prodrugs thereof.

In some embodiments, the “G” group of any of Formula (A), Formula (B), or Formula (C) is any group that is used to tailor the physical and biological properties of the molecule. Such tailoring/modifications are achieved using groups which modulate Michael acceptor chemical reactivity, acidity, basicity, lipophilicity, solubility and other physical properties of the molecule. The physical and biological properties modulated by such modifications to G include, by way of example only, enhancing chemical reactivity of Michael acceptor group, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to G allow for the tailoring of the in vivo efficacy of the compound through the modulation of, by way of example, specific and non-specific protein binding to plasma proteins and lipids and tissue distribution in vivo.

In some embodiments, the BTK inhibitor has the structure of Formula (D):

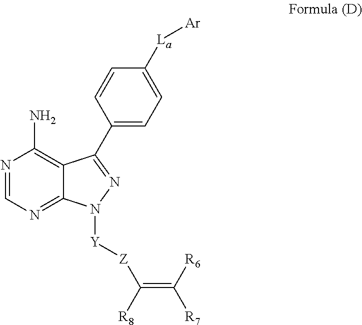

wherein

### La is CH₂, O, NH or S;

Ar is an optionally substituted aromatic carbocycle or an aromatic heterocycle;
Y is an optionally substituted alkyl, heteroalkyl, carbocycle, heterocycle, or combination thereof; Z is C(O), OC(O), NHC(O), C(S), S(O)ₓ, OS(O)ₓ, NHS(O)ₓ, where x is 1 or 2; and
R₆, R₇, and R₈ are independently selected from H, alkyl, heteroalkyl, carbocycle, heterocycle, or combinations thereof.

In some embodiments, La is O.

In some embodiments, Ar is phenyl.

In some embodiments, Z is C(O).

In some embodiments, each of R₁, R₂, and R₃ is H.

In some embodiments, provided herein is a compound of Formula (D). Formula (D) is as follows:

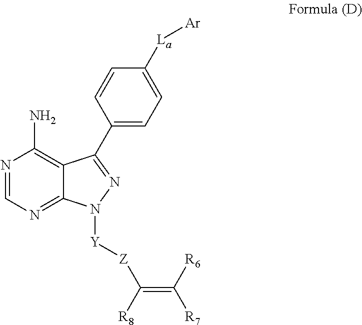

wherein:

### Lₐ is CH₂, O, NH or S;

Ar is a substituted or unsubstituted aryl, or a susbstituted or unsubstituted heteroaryl;
Y is an optionally substituted group selected from among alkyl, heteroalkyl, cycloalkyl, heterocycloalkyl, aryl, and heteroaryl;
Z is C(═O), OC(═O), NHC(═O), C(═S), S(═O)ₓ, OS(═O)ₓ, NHS(═O)ₓ, where x is 1 or 2; R₇ and R₈ are independently selected from among H, unsubstituted C₁-C₄alkyl, substituted C₁-C₄alkyl, unsubstituted C₁-C₄heteroalkyl, substituted C₁-C₄heteroalkyl, unsubstituted C₃-C₆cycloalkyl, substituted C₃-C₆cycloalkyl, unsubstituted C₂-C₆heterocycloalkyl, and substituted C₂-C₆heterocycloalkyl; or
R₇ and R₈ taken together form a bond;
R₆ is H, substituted or unsubstituted C₁-C₄alkyl, substituted or unsubstituted C₁-C₄heteroalkyl, C₁-C₆alkoxyalkyl, C₁-C₈alkylaminoalkyl, substituted or unsubstituted C₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl); and
pharmaceutically active metabolites, or pharmaceutically acceptable solvates, pharmaceutically acceptable salts, or pharmaceutically acceptable prodrugs thereof.

For any and all of the embodiments, substituents can be selected from among from a subset of the listed alternatives. For example, in some embodiments, Lₐ is CH₂, O, or NH. In other embodiments, Lₐ is O or NH. In yet other embodiments, Lₐ is O.

In some embodiments, Ar is a substituted or unsubstituted aryl. In yet other embodiments, Ar is a 6-membered aryl. In some other embodiments, Ar is phenyl.

In some embodiments, x is 2. In yet other embodiments, Z is C(═O), OC(═O), NHC(═O), S(═O)ₓ, OS(═O)ₓ, or NHS(═O)ₓ. In some other embodiments, Z is C(═O), NHC(═O), or S(═O)₂.

In some embodiments, R₇ and R₈ are independently selected from among H, unsubstituted C₁-C₄ alkyl, substituted C₁-C₄alkyl, unsubstituted C₁-C₄heteroalkyl, and substituted C₁-C₄heteroalkyl; or R₇ and R₈ taken together form a bond. In yet other embodiments, each of R₇ and R₈ is H; or R₇ and R₈ taken together form a bond.

In some embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, substituted or unsubstituted aryl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In some other embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In yet other embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl). In some embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl containing 1 or 2 N atoms), or C₁-C₄alkyl(5- or 6-membered heterocycloalkyl containing 1 or 2 N atoms).

In some embodiments, Y is an optionally substituted group selected from among alkyl, heteroalkyl, cycloalkyl, and heterocycloalkyl. In other embodiments, Y is an optionally substituted group selected from among C₁-C₆alkyl, C₁-C₆heteroalkyl, 4-, 5-, 6- or 7-membered cycloalkyl, and 4-, 5-, 6- or 7-membered heterocycloalkyl. In yet other embodiments, Y is an optionally substituted group selected from among C₁-C₆alkyl, C₁-C₆heteroalkyl, 5-, or 6-membered cycloalkyl, and 5-, or 6-membered heterocycloalkyl containing 1 or 2 N atoms. In some other embodiments, Y is a 5-, or 6-membered cycloalkyl, or a 5-, or 6-membered heterocycloalkyl containing 1 or 2 N atoms.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments the BTK inhibitor compounds of Formula (A), Formula (B), Formula (C), Formula (D), include, but are not limited to, compounds selected from the group consisting of:

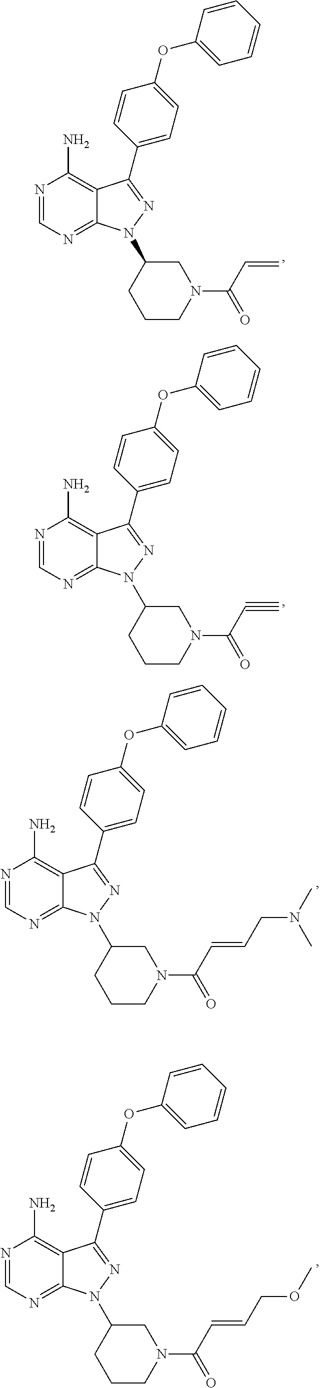

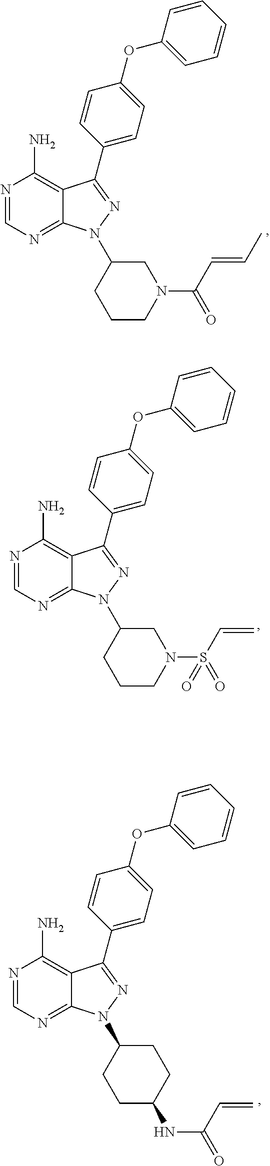 [SMILES: C/C=C/C(=O)N1CCCC(n2nc(-c3ccc(Oc4ccccc4)cc3)c3c(N)ncnc32)C1 ; C=CS(=O)(=O)N1CCCC(n2nc(-c3ccc(Oc4ccccc4)cc3)c3c(N)ncnc32)C1 ; C=CC(=O)N[C@H]1CC[C@@H](n2nc(-c3ccc(Oc4ccccc4)cc3)c3c(N)ncnc32)CC1]

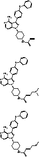 [SMILES: C#CC(=O)N[C@H]1CC[C@@H](n2nc(-c3ccc(Oc4ccccc4)cc3)c3c(N)ncnc32)CC1 ; CN(C)C/C=C/C(=O)N[C@H]1CC[C@@H](n2nc(-c3ccc(Oc4ccccc4)cc3)c3c(N)ncnc32)CC1 ; COC/C=C/C(=O)N[C@H]1CC[C@@H](n2nc(-c3ccc(Oc4ccccc4)cc3)c3c(N)ncnc32)CC1]

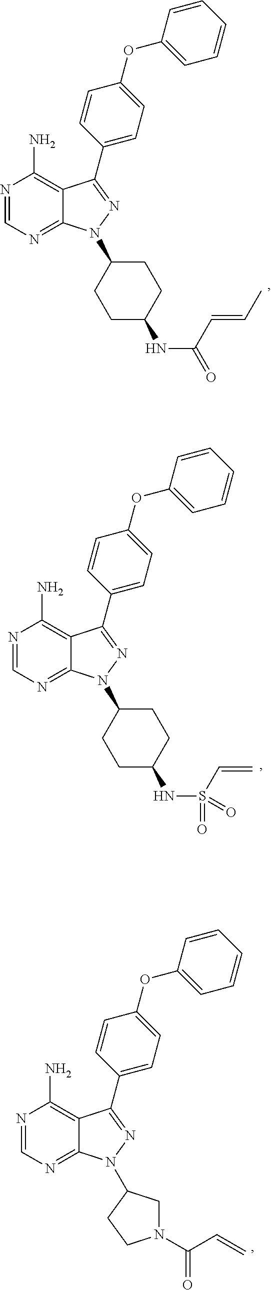 [SMILES: C/C=C/C(=O)N[C@H]1CC[C@@H](n2nc(-c3ccc(Oc4ccccc4)cc3)c3c(N)ncnc32)CC1 ; C=CS(=O)(=O)N[C@H]1CC[C@@H](n2nc(-c3ccc(Oc4ccccc4)cc3)c3c(N)ncnc32)CC1 ; C=CC(=O)N1CCC(n2nc(-c3ccc(Oc4ccccc4)cc3)c3c(N)ncnc32)C1]

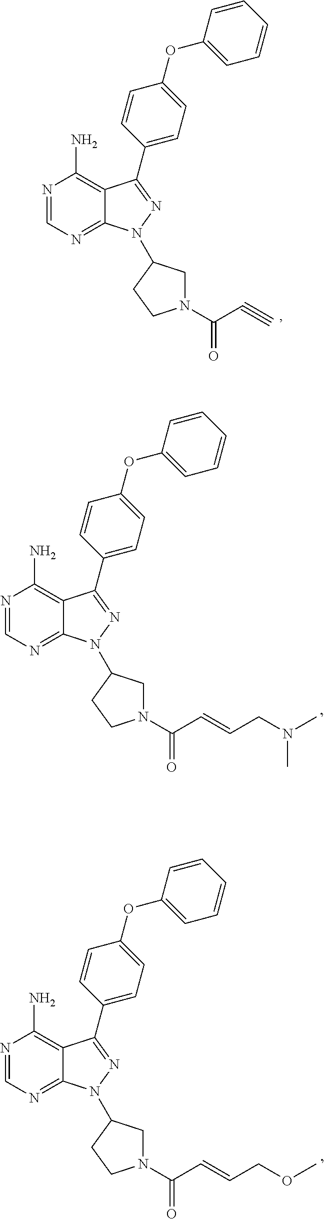 [SMILES: C#CC(=O)N1CCC(n2nc(-c3ccc(Oc4ccccc4)cc3)c3c(N)ncnc32)C1 ; CN(C)C/C=C/C(=O)N1CCC(n2nc(-c3ccc(Oc4ccccc4)cc3)c3c(N)ncnc32)C1 ; COC/C=C/C(=O)N1CCC(n2nc(-c3ccc(Oc4ccccc4)cc3)c3c(N)ncnc32)C1]

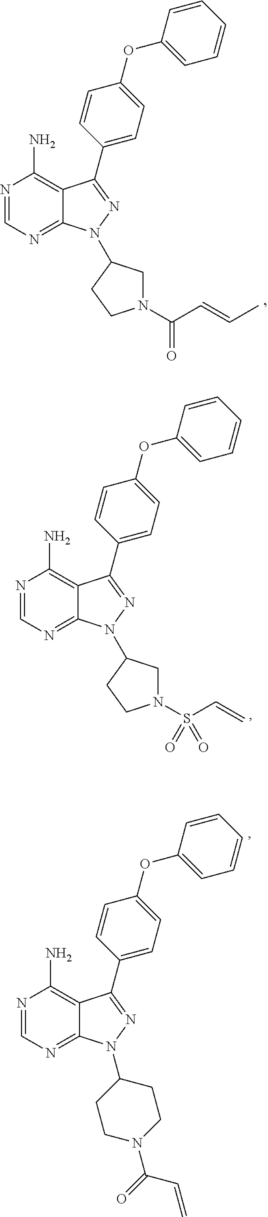 [SMILES: C/C=C/C(=O)N1CCC(n2nc(-c3ccc(Oc4ccccc4)cc3)c3c(N)ncnc32)C1 ; C=CS(=O)(=O)N1CCC(n2nc(-c3ccc(Oc4ccccc4)cc3)c3c(N)ncnc32)C1 ; C=CC(=O)N1CCC(n2nc(-c3ccc(Oc4ccccc4)cc3)c3c(N)ncnc32)CC1]

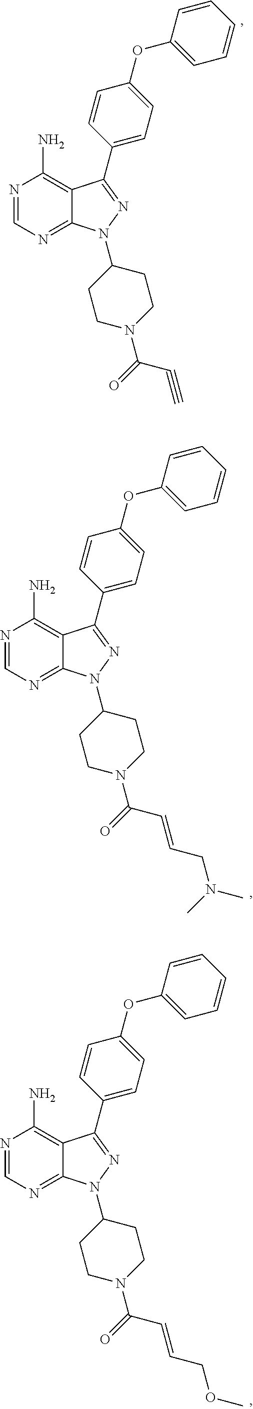

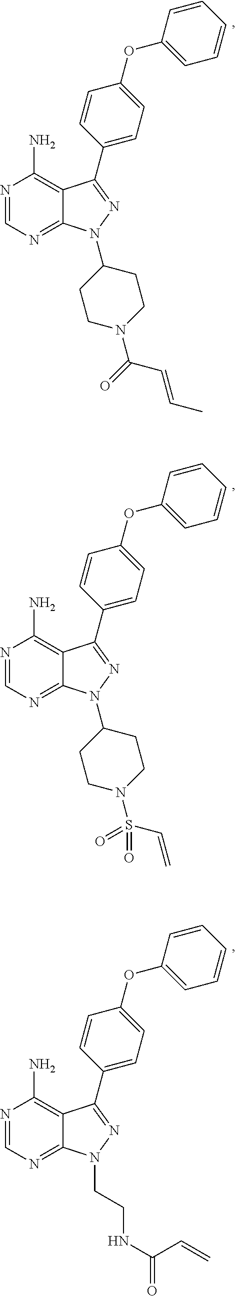 [SMILES: C/C=C/C(=O)N1CCC(n2nc(-c3ccc(Oc4ccccc4)cc3)c3c(N)ncnc32)CC1 ; C=CS(=O)(=O)N1CCC(n2nc(-c3ccc(Oc4ccccc4)cc3)c3c(N)ncnc32)CC1 ; C=CC(=O)NCCn1nc(-c2ccc(Oc3ccccc3)cc2)c2c(N)ncnc21]

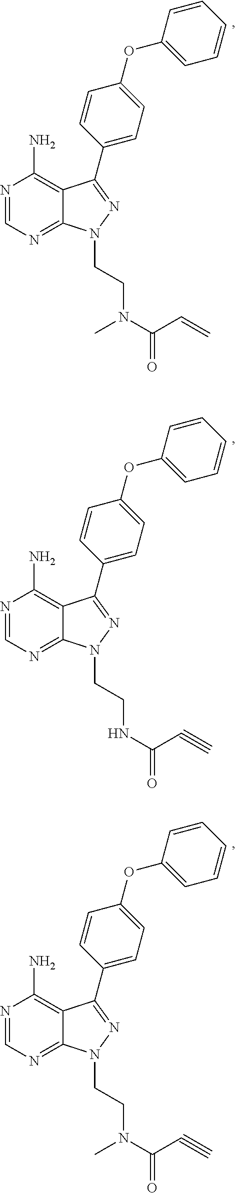 [SMILES: C=CC(=O)N(C)CCn1nc(-c2ccc(Oc3ccccc3)cc2)c2c(N)ncnc21 ; C#CC(=O)NCCn1nc(-c2ccc(Oc3ccccc3)cc2)c2c(N)ncnc21 ; C#CC(=O)N(C)CCn1nc(-c2ccc(Oc3ccccc3)cc2)c2c(N)ncnc21]

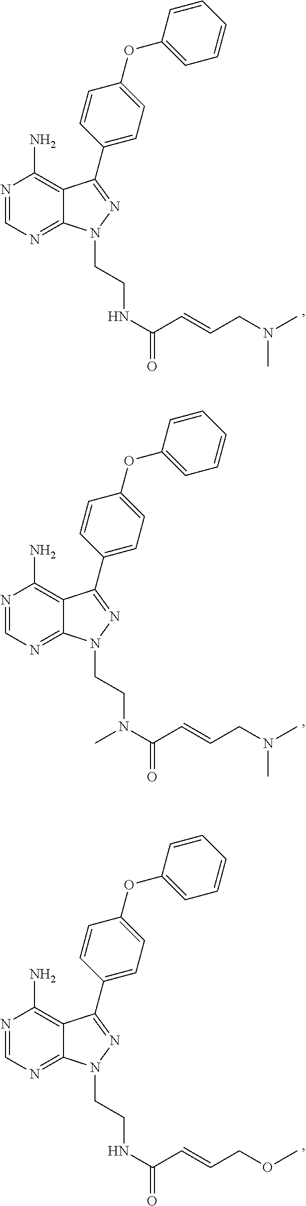 [SMILES: CN(C)C/C=C/C(=O)NCCn1nc(-c2ccc(Oc3ccccc3)cc2)c2c(N)ncnc21 ; CN(C)C/C=C/C(=O)N(C)CCn1nc(-c2ccc(Oc3ccccc3)cc2)c2c(N)ncnc21 ; COC/C=C/C(=O)NCCn1nc(-c2ccc(Oc3ccccc3)cc2)c2c(N)ncnc21]

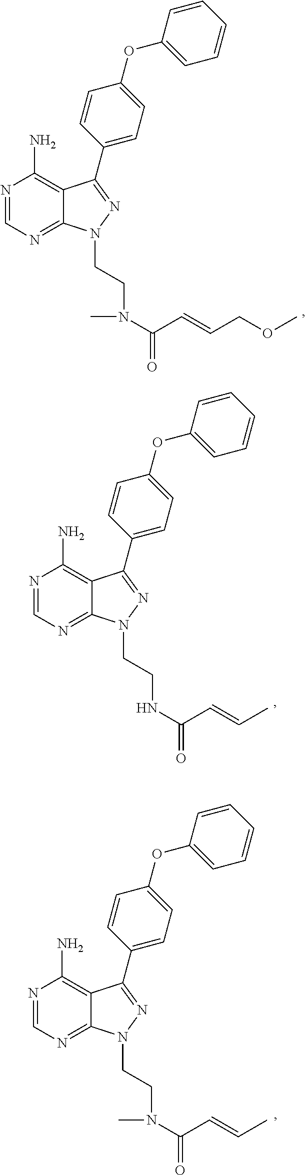 [SMILES: COC/C=C/C(=O)N(C)CCn1nc(-c2ccc(Oc3ccccc3)cc2)c2c(N)ncnc21 ; C/C=C/C(=O)NCCn1nc(-c2ccc(Oc3ccccc3)cc2)c2c(N)ncnc21 ; C/C=C/C(=O)N(C)CCn1nc(-c2ccc(Oc3ccccc3)cc2)c2c(N)ncnc21]

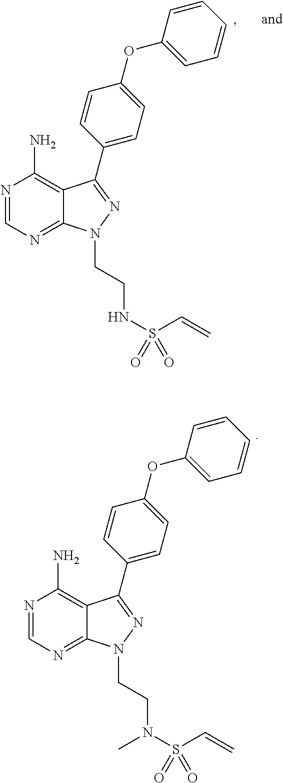 [SMILES: C=CS(=O)(=O)NCCn1nc(-c2ccc(Oc3ccccc3)cc2)c2c(N)ncnc21 ; C=CS(=O)(=O)N(C)CCn1nc(-c2ccc(Oc3ccccc3)cc2)c2c(N)ncnc21]

In some embodiments, the BTK inhibitor compounds are selected from among:

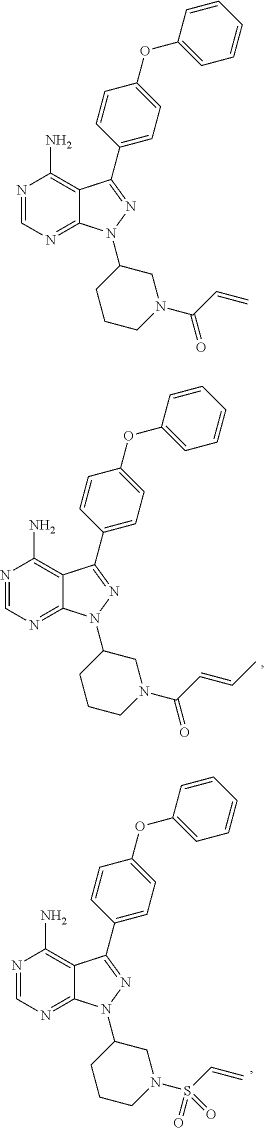

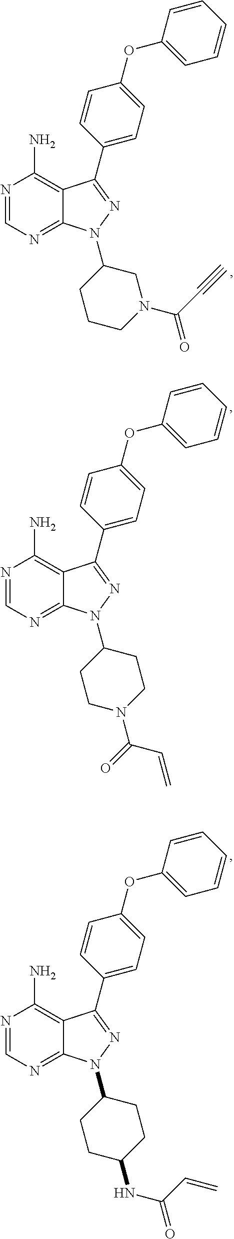

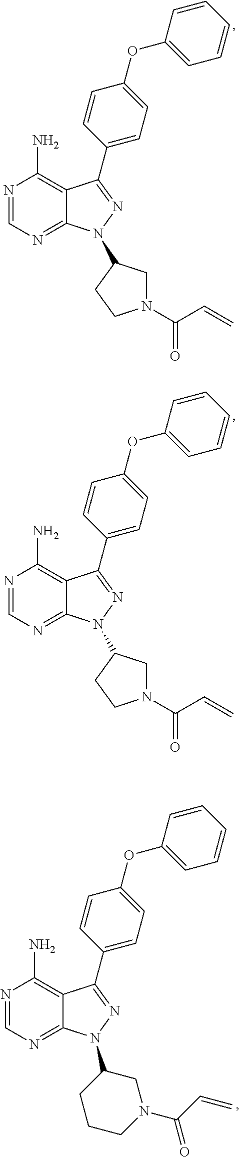

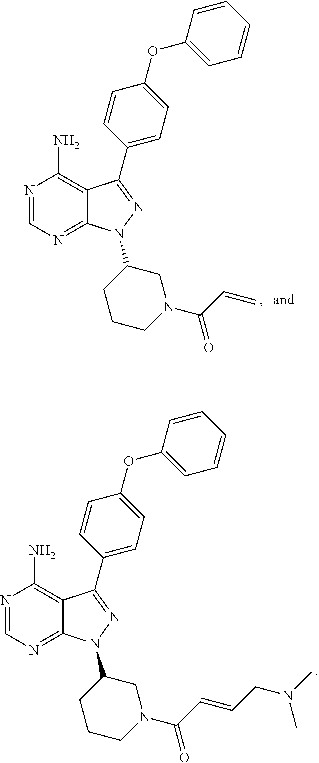

In some embodiments, the BTK inhibitor compounds are selected from among: 1-(3-(4-amino-3-(4-phenoxyphenyl)-1H-pyrazolo[3,4-d]pyrimidin-1-yl)piperidin-1-yl)prop-2-en-1-one (Compound 4); (E)-1-(3-(4-amino-3-(4-phenoxyphenyl)-1H-pyrazolo[3,4-d]pyrimidin-1-yl)piperidin-1-yl)but-2-en-1-one (Compound 5); 1-(3-(4-amino-3-(4-phenoxyphenyl)-1H-pyrazolo[3,4-d]pyrimidin-1-yl)piperidin-1-yl)sulfonylethene (Compound 6); 1-(3-(4-amino-3-(4-phenoxyphenyl)-1H-pyrazolo[3,4-d]pyrimidin-1-yl)piperidin-1-yl)prop-2-yn-1-one (Compound 8); 1-(4-(4-amino-3-(4-phenoxyphenyl)-1H-pyrazolo[3,4-d]pyrimidin-1-yl)piperidin-1-yl)prop-2-en-1-one (Compound 9); N-((1s,4s)-4-(4-amino-3-(4-phenoxyphenyl)-1H-pyrazolo[3,4-d]pyrimidin-1-yl)cyclohexyl)acrylamide (Compound 10); 1-((R)-3-(4-amino-3-(4-phenoxyphenyl)-1H-pyrazolo[3,4-d]pyrimidin-1-yl)pyrrolidin-1-yl)prop-2-en-1-one (Compound 11); 1-((S)-3-(4-amino-3-(4-phenoxyphenyl)-1H-pyrazolo[3,4-d]pyrimidin-1-yl)pyrrolidin-1-yl)prop-2-en-1-one (Compound 12); 1-((R)-3-(4-amino-3-(4-phenoxyphenyl)-1H-pyrazolo[3,4-d]pyrimidin-1-yl)piperidin-1-yl)prop-2-en-1-one (Compound 13); 1-((S)-3-(4-amino-3-(4-phenoxyphenyl)-1H-pyrazolo[3,4-d]pyrimidin-1-yl)piperidin-1-yl)prop-2-en-1-one (Compound 14); and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one (Compound 15).

Throughout the specification, groups and substituents thereof can be chosen by one skilled in the field to provide stable moieties and compounds.

The compounds of any of Formula (A), or Formula (B), or Formula (C), or Formula (D) can irreversibly inhibit Btk and may be used to treat patients suffering from Bruton's tyrosine kinase-dependent or Bruton's tyrosine kinase mediated conditions or diseases, including, but not limited to, cancer, autoimmune and other inflammatory diseases.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-1(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

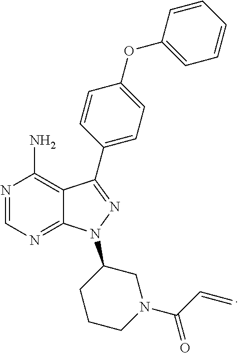 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), and GDC-0853 (Genentech).

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

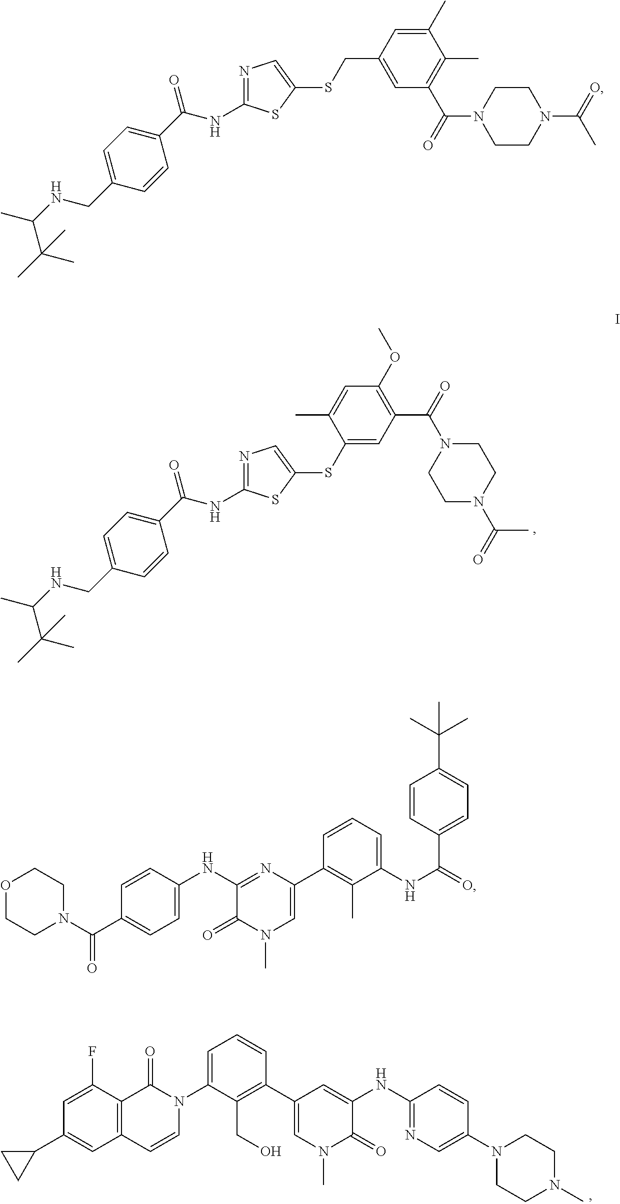

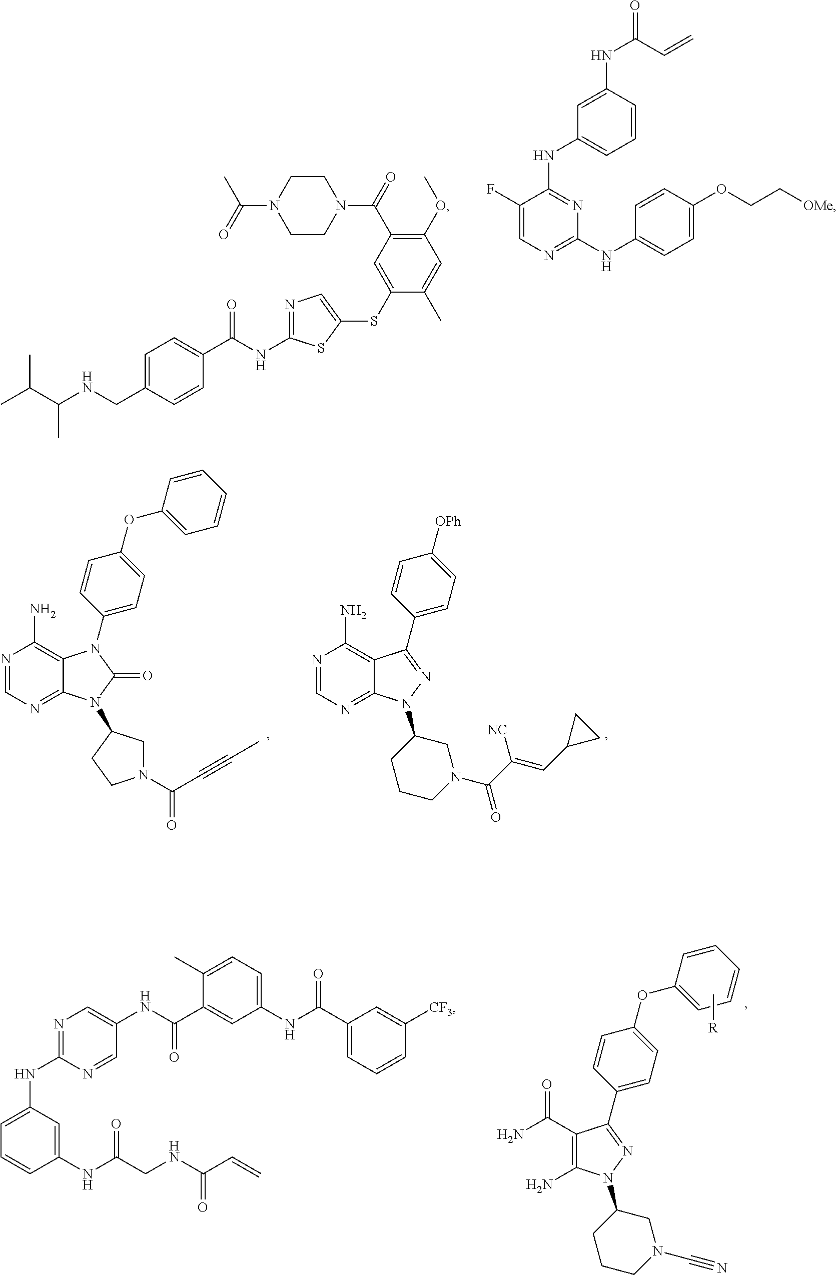

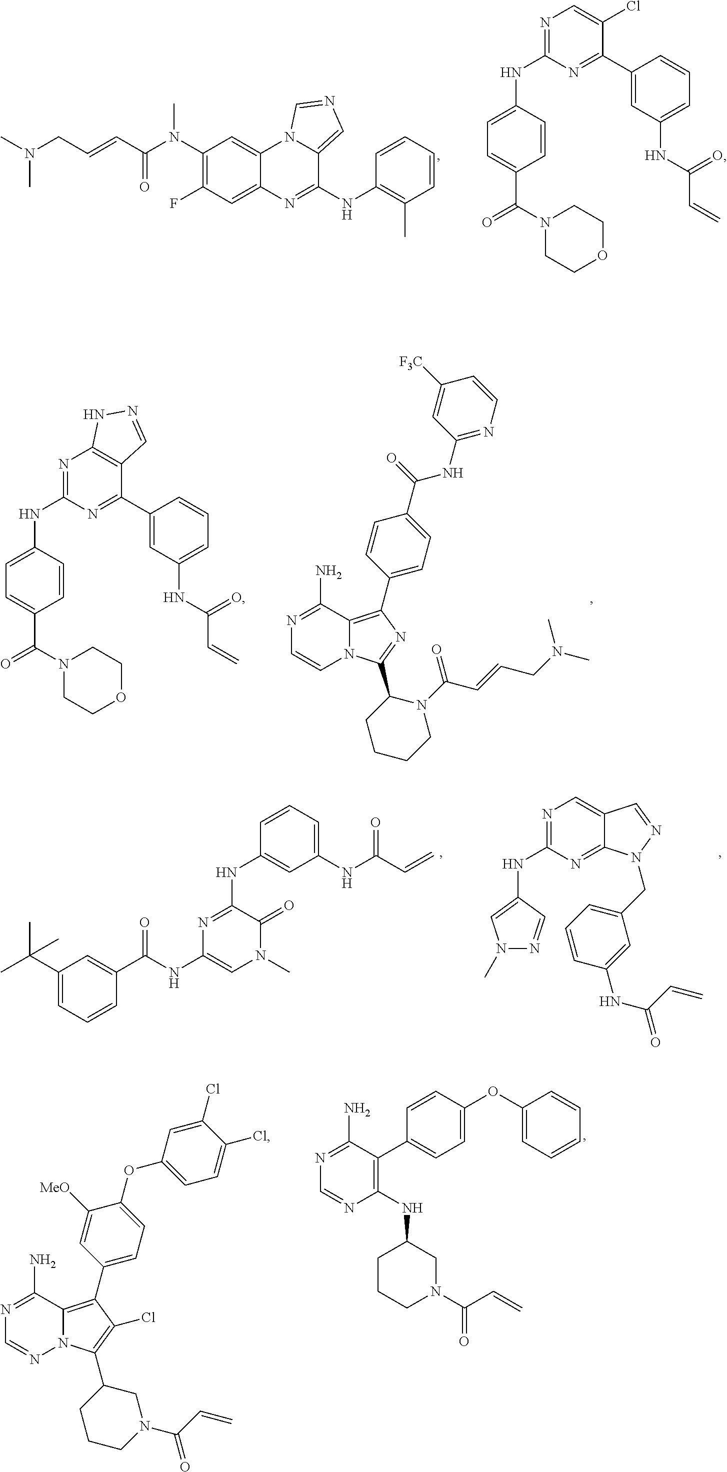

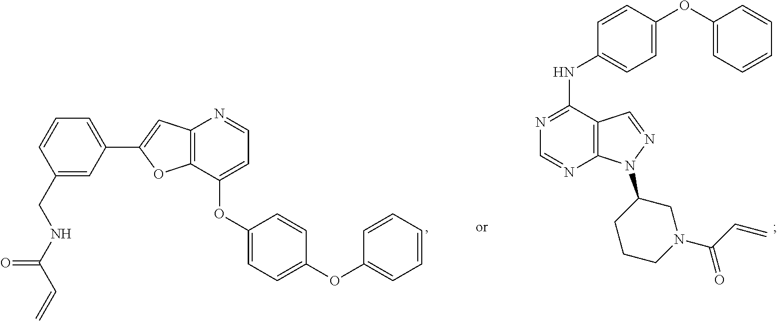 [SMILES: C=CC(=O)NCc1cccc(-c2cc3nccc(Oc4ccc(Oc5ccccc5)cc4)c3o2)c1 ; C=CC(=O)N1CCC[C@@H](n2ncc3c(Nc4ccc(Oc5ccccc5)cc4)ncnc32)C1]

or a pharmaceutically acceptable salt thereof.

### ITK Inhibitors

In some embodiments, ACK inhibitor is an ITK inhibitor. In some embodiments, the ITK inhibitor covalently binds to Cysteine 442 of ITK. In some embodiments, the ITK inhibitor is an ITK inhibitor compound described in WO2002/0500071, which is incorporated by reference in its entirety. In some embodiments, the ITK inhibitor is an ITK inhibitor compound described in WO2005/070420, which is incorporated by reference in its entirety. In some embodiments, the ITK inhibitor is an ITK inhibitor compound described in WO2005/079791, which is incorporated by reference in its entirety. In some embodiments, the ITK inhibitor is an ITK inhibitor compound described in WO2007/076228, which is incorporated by reference in its entirety. In some embodiments, the ITK inhibitor is an ITK inhibitor compound described in WO2007/058832, which is incorporated by reference in its entirety. In some embodiments, the ITK inhibitor is an ITK inhibitor compound described in WO2004/016610, which is incorporated by reference in its entirety. In some embodiments, the ITK inhibitor is an ITK inhibitor compound described in WO2004/016611, which is incorporated by reference in its entirety. In some embodiments, the ITK inhibitor is an ITK inhibitor compound described in WO2004/016600, which is incorporated by reference in its entirety. In some embodiments, the ITK inhibitor is an ITK inhibitor compound described in WO2004/016615, which is incorporated by reference in its entirety. In some embodiments, the ITK inhibitor is an ITK inhibitor compound described in WO2005/026175, which is incorporated by reference in its entirety. In some embodiments, the ITK inhibitor is an ITK inhibitor compound described in WO2006/065946, which is incorporated by reference in its entirety. In some embodiments, the ITK inhibitor is an ITK inhibitor compound described in WO2007/027594, which is incorporated by reference in its entirety. In some embodiments, the ITK inhibitor is an ITK inhibitor compound described in WO2007/017455, which is incorporated by reference in its entirety. In some embodiments, the ITK inhibitor is an ITK inhibitor compound described in WO2008/025820, which is incorporated by reference in its entirety. In some embodiments, the ITK inhibitor is an ITK inhibitor compound described in WO2008/025821, which is incorporated by reference in its entirety. In some embodiments, the ITK inhibitor is an ITK inhibitor compound described in WO2008/025822, which is incorporated by reference in its entirety. In some embodiments, the ITK inhibitor is an ITK inhibitor compound described in WO2011/017219, which is incorporated by reference in its entirety. In some embodiments, the ITK inhibitor is an ITK inhibitor compound described in WO2011/090760, which is incorporated by reference in its entirety. In some embodiments, the ITK inhibitor is an ITK inhibitor compound described in WO2009/158571, which is incorporated by reference in its entirety. In some embodiments, the ITK inhibitor is an ITK inhibitor compound described in WO2009/051822, which is incorporated by reference in its entirety. In some embodiments, the Itk inhibitor is an Itk inhibitor compound described in US20110281850, which is incorporated by reference in its entirety. In some embodiments, the Itk inhibitor is an Itk inhibitor compound described in WO2014/082085, which is incorporated by reference in its entirety. In some embodiments, the Itk inhibitor is an Itk inhibitor compound described in WO2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO2014/105958, which is incorporated by reference in its entirety. In some embodiments, the Itk inhibitor is an Itk inhibitor compound described in US2014/0256704, which is incorporated by reference in its entirety. In some embodiments, the Itk inhibitor is an Itk inhibitor compound described in US20140315909, which is incorporated by reference in its entirety. In some embodiments, the Itk inhibitor is an Itk inhibitor compound described in US20140303161, which is incorporated by reference in its entirety. In some embodiments, the Itk inhibitor is an Itk inhibitor compound described in WO2014/145403, which is incorporated by reference in its entirety.

In some embodiments, the ITK inhibitor has a structure selected from:

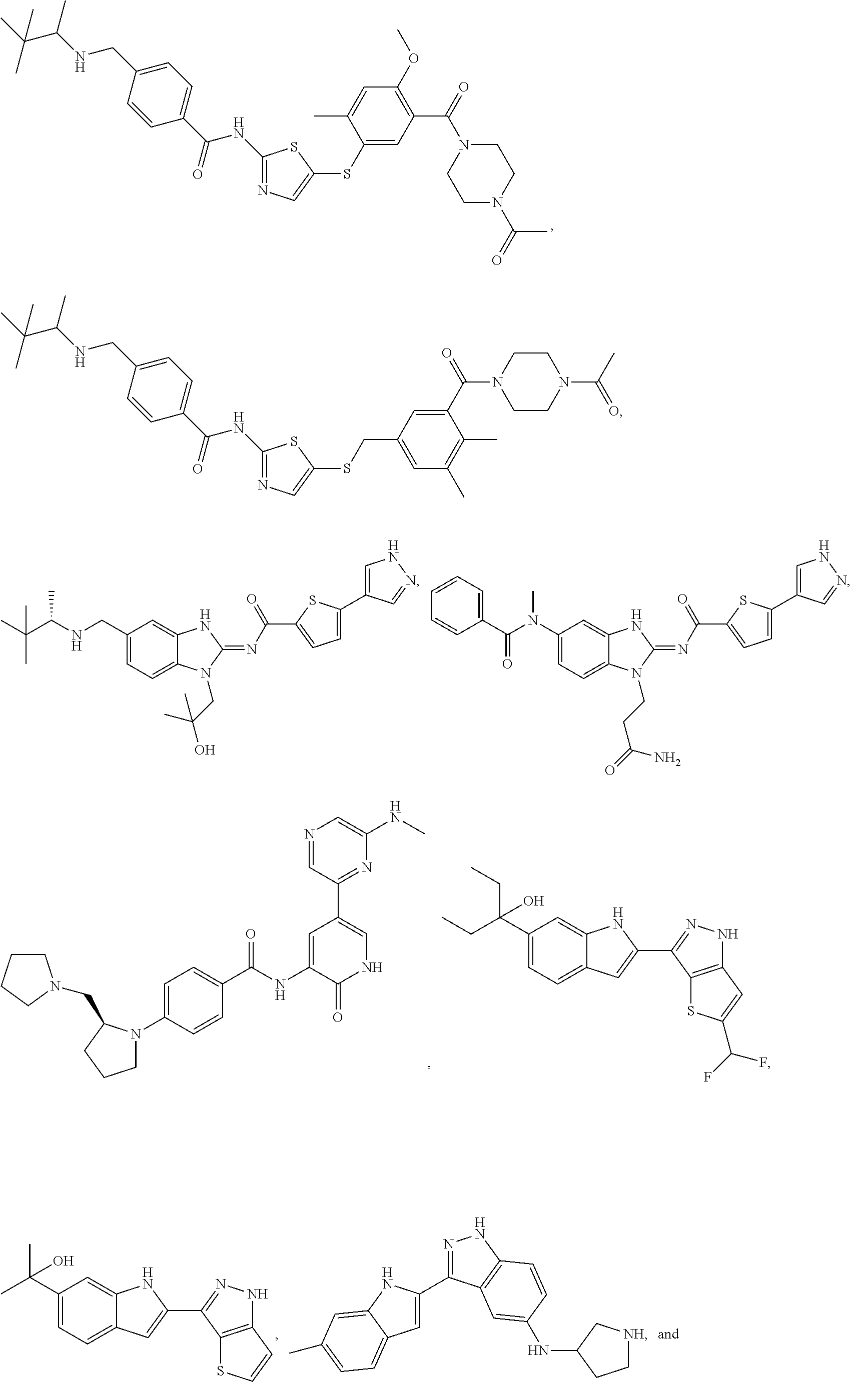

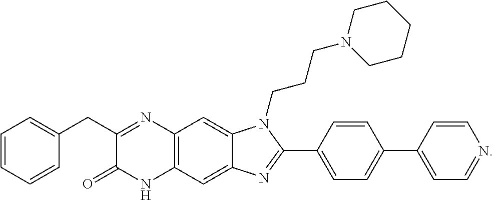 [SMILES: O=c1[nH]c2cc3nc(-c4ccc(-c5ccncc5)cc4)n(CCCN4CCCCC4)c3cc2nc1Cc1ccccc1]

### Diagnostic and Therapeutic Methods

In some embodiments, methods are provided for treating DLBCL by pre-selecting patients who express elevated levels of CCL3 and/or CCL4, thereby increasing the likelihood of a response, in the patient, to therapeutics that inhibit BTK. In some embodiments, provided are methods for treating DLBCL, in a patient in need thereof, by assessing whether the patient expresses elevated levels of CCL3 and/or CCL4, thereby increasing the likelihood of a response, in the patient, to therapeutics that inhibit BTK. In some embodiments, provided are methods for treating DLBCL, in a patient in need thereof, by assessing whether the patient exhibits a change in expression levels of CCL3 and/or CCL4 following treatment with a therapeutic that inhibits BTK. In some embodiments, the BTK inhibitor is ibrutinib.

In some embodiments, the DLBCL is ABC subtype DLBCL. In some embodiments, the Activated B cell-like (ABC) subtype of diffuse large B-cell lymphoma (DLBCL) is characterized by a CD79B mutation. In some embodiments, the CD79B mutation is a mutation of the immunoreceptor tyrosine-based activation motif (ITAM) signaling module. In some embodiments, the CD79B mutation is a mis sense mutation of the first immunoreceptor tyrosine-based activation motif (ITAM) tyrosine. In some embodiments, the CD79B mutation increases surface BCR expression and attenuates Lyn kinase activity. In some embodiments, the Activated B cell-like (ABC) subtype of diffuse large B-cell lymphoma (DLBCL) is characterized by a CD79A mutation. In some embodiments, the CD79A mutation is in the immunoreceptor tyrosine-based activation motif (ITAM) signaling module. In some embodiments, the CD79A mutation is a splice-donor-site mutation of the immunoreceptor tyrosine-based activation motif (ITAM) signaling module. In some embodiments, the CD79A mutation deletes the immunoreceptor tyrosine-based activation motif (ITAM) signaling module. In some embodiments, the Activated B cell-like (ABC) subtype of diffuse large B-cell lymphoma (DLBCL) is characterized by a mutation in MyD88, A20, or a combination thereof. In some embodiments, the MyD88 mutation is the amino acid substitution L265P in the MYD88 Toll/IL-1 receptor (TIR) domain.

In some embodiments, an exemplary method for treating DLBCL in a patient in need thereof comprises: (a) determining an expression level of CCL3 and/or CCL4 in a sample (e.g., a serum sample) from the patient; and (b) administering, to the patient, a therapeutically effective amount of a BTK inhibitor if the expression of CCL3 and/or CCL4 is increased relative to a control or reference level. In some embodiments, a reference level is level of expression of CCL3 and/or CCL4 in a normal patient (e.g., a patient without DLBCL).

In some embodiments, an exemplary method for treating DLBCL in a patient in need thereof comprises: (a) determining an expression level of CCL3 and/or CCL4 in a sample (e.g., a serum sample) from the patient; and (b) administering, to the patient, a therapeutically effective amount of ibrutinib if the expression of CCL3 and/or CCL4 is increased relative to a control or reference level. In some embodiments, a reference level is level of expression of CCL3 and/or CCL4 in a normal patient (e.g., a patient without DLBCL).

In some embodiments, methods are provided for evaluating a response to a BTK inhibitor in a patient having DLBCL which comprises determining an expression level of CCL3 and/or CCL4 in a sample (e.g., a serum sample) from the patient following administration of a therapeutically effective amount of a BTK inhibitor, wherein if a patient sample exhibits a decrease in the levels of CCL3 and/or CCL4 expression relative to the control or reference level, a favorable outcome for treatment with a BTK inhibitor is predicted. In some embodiments, methods are provided for evaluating a response to a BTK inhibitor in a patient having DLBCL which comprises determining an expression level of CCL3 and/or CCL4 in a sample (e.g., a serum sample) from the patient following administration of a therapeutically effective amount of a BTK inhibitor, wherein if a patient sample exhibits a decrease in the levels of CCL3 and/or CCL4 expression relative to the control or reference level, the patient is characterized as responding to treatment with the BTK inhibitor. In some embodiments, a reference level is level of expression of CCL3 and/or CCL4 in a normal patient (e.g., a patient without DLBCL). In some embodiments, level of CCL3 expression following treatment with a BTK inhibitor decreases by 5%, 10%, 15%, 20%, 25%, 30%, 35%, 40%, 45%, 50%, 55%, 60%, 65%, 70%, 75%, 80%, 85%, 90%, 95% 99% or greater following treatment with ibrutinib. In some embodiments, the level of CCL3 expression following treatment decreases to the level of expression in a normal patient (i.e., normalizes) following treatment with a BTK inhibitor. In some embodiments, level of CCL4 expression following treatment with a BTK inhibitor decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the level of CCL4 expression following treatment with ibrutinib decreases to the level of expression in a normal patient (i.e., normalizes) following treatment with a BTK inhibitor. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor.

In some embodiments, methods are provided for evaluating a response to ibrutinib in a patient having DLBCL which comprises determining an expression level of CCL3 and/or CCL4 in a sample (e.g., a serum sample) from the patient following administration of a therapeutically effective amount of ibrutinib, wherein if a patient sample exhibits a decrease in the levels of CCL3 and/or CCL4 expression relative to the control or reference level, a favorable outcome for treatment with ibrutinib is predicted. In some embodiments, methods are provided for evaluating a response to ibrutinib in a patient having DLBCL which comprises determining an expression level of CCL3 and/or CCL4 in a sample (e.g., a serum sample) from the patient following administration of a therapeutically effective amount of ibrutinib, wherein if a patient sample exhibits a decrease in the levels of CCL3 and/or CCL4 expression relative to the control or reference level, the patient is characterized as responding to treatment with ibrutinib. In some embodiments, a reference level is level of expression of CCL3 and/or CCL4 in a normal patient (e.g., a patient without DLBCL). In some embodiments, level of CCL3 expression following treatment with ibrutinib decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the level of CCL3 expression following treatment decreases to the level of expression in a normal patient (i.e., normalizes) following treatment with ibrutinib. In some embodiments, level of CCL4 expression following treatment decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the level of CCL4 expression following treatment with ibrutinib decreases to the level of expression in a normal patient (i.e., normalizes) following treatment with ibrutinib. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with ibrutinib.

In some embodiments, an exemplary method for treating DLBCL in a patient in need thereof comprises: (a) determining an expression level of CCL3 and/or CCL4 in a sample (e.g., a serum sample) from the patient following administration of a therapeutically effective amount of a BTK inhibitor; and (b) modifying, discontinuing, or continuing the treatment based on the expression of CCL3 and/or CCL4 relative to a control or reference level. In some embodiments, a reference level is level of expression of CCL3 and/or CCL4 in a sample (e.g., a serum sample) taken from the patient prior to administration of the therapeutically effective amount of the BTK inhibitor. In some embodiments, the treatment regimen is continued. In some embodiments, the treatment regimen is modified. In some embodiments, the dosage of the BTK inhibitor is increased. In some embodiments, the dosage of the BTK inhibitor is decreased. In some embodiments, the dosage of the BTK inhibitor not modified. In some embodiments, the frequency of administration of the BTK inhibitor is increased. In some embodiments, the frequency of administration of the BTK inhibitor is decreased. In some embodiments, the frequency of administration of the BTK inhibitor is not modified. In some embodiments, the timing of administration of the BTK inhibitor is modified (e.g., time of day or time relative to administration of other therapeutic agents). In some embodiments, the timing of administration of the BTK inhibitor is not modified. In some embodiments, an additional therapeutic agent is administered. In some embodiments, an additional anti-cancer agent is administered. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor.

In some embodiments, an exemplary method for treating DLBCL in a patient in need thereof comprises: (a) determining an expression level of CCL3 and/or CCL4 in a sample (e.g., a serum sample) from the patient following administration of a therapeutically effective amount of ibrutinib; and (b) modifying, discontinuing, or continuing the treatment based on the expression of CCL3 and/or CCL4 relative to a control or reference level. In some embodiments, a reference level is level of expression of CCL3 and/or CCL4 in a sample (e.g., a serum sample) taken from the patient prior to administration of the therapeutically effective amount of ibrutinib. In some embodiments, the treatment regimen is continued. In some embodiments, the treatment regimen is modified. In some embodiments, the dosage of ibrutinib is increased. In some embodiments, the dosage of ibrutinib is decreased. In some embodiments, the dosage of ibrutinib not modified. In some embodiments, the frequency of administration of ibrutinib is increased. In some embodiments, the frequency of administration of ibrutinib is decreased. In some embodiments, the frequency of administration of ibrutinib is not modified. In some embodiments, the timing of administration of ibrutinib is modified (e.g., time of day or time relative to administration of other therapeutic agents). In some embodiments, the timing of administration of ibrutinib is not modified. In some embodiments, an additional therapeutic agent is administered. In some embodiments, an additional anti-cancer agent is administered. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor.

In some embodiments, an exemplary method for treating DLBCL in a patient in need thereof comprises: (a) administering a treatment comprising a therapeutically effective amount of a BTK inhibitor; (b) determining an expression level of CCL3 and/or CCL4 in a sample from the patient following administration of the treatment; and (c) continuing the treatment if the expression of CCL3 and/or CCL4 is decreased by a predetermined amount relative to the expression level of CCL3 and/or CCL4 prior to treatment. In some embodiments, the predetermined amount is a decrease in the level of CCL3 and/or CCL4 expression by 3% 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the dosage of ibrutinib is increased. In some embodiments, the dosage of ibrutinib is decreased. In some embodiments, the dosage of ibrutinib not modified. In some embodiments, the frequency of administration of ibrutinib is increased. In some embodiments, the frequency of administration of ibrutinib is decreased. In some embodiments, the frequency of administration of ibrutinib is not modified. In some embodiments, the timing of administration of ibrutinib is modified (e.g., time of day or time relative to administration of other therapeutic agents). In some embodiments, the timing of administration of ibrutinib is not modified. In some embodiments, an additional therapeutic agent is administered. In some embodiments, an additional anti-cancer agent is administered. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor.

In some embodiments, an exemplary method for treating DLBCL in a patient in need thereof comprises: (a) administering a treatment comprising a therapeutically effective amount of ibrutinib; (b) determining an expression level of CCL3 and/or CCL4 in a sample from the patient following administration of the treatment; and (c) continuing the treatment if the expression of CCL3 and/or CCL4 is decreased by a predetermined amount relative to the expression level of CCL3 and/or CCL4 prior to treatment. In some embodiments, the predetermined amount is a decrease in the level of CCL3 and/or CCL4 expression by 3% 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the dosage of ibrutinib is increased. In some embodiments, the dosage of ibrutinib is decreased. In some embodiments, the dosage of ibrutinib not modified. In some embodiments, the frequency of administration of ibrutinib is increased. In some embodiments, the frequency of administration of ibrutinib is decreased. In some embodiments, the frequency of administration of ibrutinib is not modified. In some embodiments, the timing of administration of ibrutinib is modified (e.g., time of day or time relative to administration of other therapeutic agents). In some embodiments, the timing of administration of ibrutinib is not modified. In some embodiments, an additional therapeutic agent is administered. In some embodiments, an additional anti-cancer agent is administered. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with ibrutinib.

In some embodiments, an exemplary method for treating DLBCL in a patient in need thereof comprises: (a) administering a treatment comprising a therapeutically effective amount of a BTK inhibitor; (b) determining an expression level of CCL3 and/or CCL4 in a sample from the patient following administration of the treatment; and (c) discontinuing the treatment if the expression of CCL3 and/or CCL4 is not decreased by a predetermined amount relative to the expression level of CCL3 and/or CCL4 prior to treatment. In some embodiments, the predetermined amount is a decrease in the level of CCL3 and/or CCL4 expression by 3% 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor.

In some embodiments, an exemplary method for treating DLBCL in a patient in need thereof comprises: (a) administering a treatment comprising a therapeutically effective amount of ibrutinib; (b) determining an expression level of CCL3 and/or CCL4 in a sample from the patient following administration of the treatment; and (c) discontinuing the treatment if the expression of CCL3 and/or CCL4 is not decreased by a predetermined amount relative to the expression level of CCL3 and/or CCL4 prior to treatment. In some embodiments, the predetermined amount is a decrease in the level of CCL3 and/or CCL4 expression by 3% 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with ibrutinib.

In some embodiments, methods are provided for predicting a response to a BTK inhibitor in a patient having DLBCL which comprises determining an expression level of CCL3 and/or CCL4 in a sample (e.g., a serum sample) from the patient prior to administering the BTK inhibitor and comparing it to a control or reference, wherein if a patient sample exhibits high levels of CCL3 and/or CCL4 expression relative to the control or reference level, a favorable outcome for treatment with a BTK inhibitor is predicted. In some embodiments, a reference level is level of expression of CCL3 and/or CCL4 in a normal patient (e.g., a patient without DLBCL).

In some embodiments, methods are provided for predicting a response to ibrutinib in a patient having DLBCL which comprises determining an expression level of CCL3 and/or CCL4 in a sample (e.g., a serum sample) from the patient prior to administering ibrutinib and comparing it to a control or reference, wherein if a patient sample exhibits high levels of CCL3 and/or CCL4 expression relative to the control or reference level, a favorable outcome for treatment with ibrutinib is predicted. In some embodiments, a reference level is level of expression of CCL3 and/or CCL4 in a normal patient (e.g., a patient without DLBCL).

In some embodiments, methods are provided for treating DLBCL in a selected patient comprising administering to the selected patient a therapeutically effective amount of a BTK inhibitor in an amount effective to treat DLBCL, wherein said selected patient has a high expression level of CCL3 and/or CCL4 prior to administration of the BTK inhibitor and wherein high expression levels of CCL3 and/or CCL4 relative to normal indicates that the selected patient would benefit from continued treatment with the BTK inhibitor.

In some embodiments, methods are provided for treating DLBCL in a selected patient comprising administering to the selected patient a therapeutically effective amount of ibrutinib in an amount effective to treat DLBCL, wherein said selected patient has a high expression level of CCL3 and/or CCL4 prior to administration of ibrutinib and wherein high expression levels of CCL3 and/or CCL4 relative to normal indicates that the selected patient would benefit from continued treatment with ibrutinib.

In some embodiments, methods are provided for treating DLBCL in a selected patient comprising administering to the selected patient a therapeutically effective amount of a BTK inhibitor in an amount effective to treat DLBCL, wherein the selected subject has high expression levels of CCL3 and/or CCL4 relative to normal prior to administration of the BTK inhibitor and wherein a decrease in the expression levels of CCL3 and/or CCL4 following treatment with the BTK inhibitor indicates that the selected subject would benefit from continued treatment with the BTK inhibitor.

In some embodiments, methods are provided for treating DLBCL in a selected patient comprising administering to the selected patient a therapeutically effective amount of ibrutinib in an amount effective to treat DLBCL, wherein the selected subject has high expression levels of CCL3 and/or CCL4 relative to normal prior to administration of ibrutinib and wherein a decrease in the expression levels of CCL3 and/or CCL4 following treatment with ibrutinib indicates that the selected subject would benefit from continued treatment with ibrutinib.

In some embodiments, methods are provided for identifying a patient that is likely to respond therapeutically to treatment with a BTK inhibitor, wherein the method comprises: (a), measuring in a sample (e.g., a serum sample) obtained from a patient having DLBCL or is suspected of having DLBCL the expression level of CCL3 and/or CCL4; (b) comparing the level obtained in step (a) to the level of expression of said CCL3 and/or CCL4 in a control sample, wherein an increase in the level of CCL3 and/or CCL4 measured in step (a) relative to the control sample indicates that the patient will respond therapeutically to treatment with the BTK inhibitor, whereas the level of CCL3 and/or CCL4 that is not increased or is decreased relative to the control sample indicates that the patient is not likely to respond to treatment or be resistant to treatment with the BTK inhibitor.

In some embodiments, methods are provided for identifying a patient that is likely to respond therapeutically to treatment with ibrutinib, wherein the method comprises: (a), measuring in a sample (e.g., a serum sample) obtained from a patient having DLBCL or is suspected of having DLBCL the expression level of CCL3 and/or CCL4; (b) comparing the level obtained in step (a) to the level of expression of said CCL3 and/or CCL4 in a control sample, wherein an increase in the level of CCL3 and/or CCL4 measured in step (a) the control sample indicates that the patient will respond therapeutically to treatment with ibrutinib, whereas the level of CCL3 and/or CCL4 that is not increased or is decreased relative to the control sample indicates that the patient is not likely to respond to treatment or be resistant to treatment with ibrutinib.

In some embodiments, methods are provided for predicting whether a patient will respond therapeutically to a method of treating DLBCL comprising administering a BTK inhibitor, wherein the method comprises: (a) measuring in a sample (e.g., a serum sample) obtained from the patient the expression level of CCL3 and/or CCL4; (b) comparing the level obtained in step (a) to the level of expression of said CCL3 and/or CCL4 in a control sample, wherein an increase in the level of CCL3 and/or CCL4 measured in step (a) indicates that the patient will respond therapeutically to treatment with the BTK inhibitor, whereas the level of CCL3 and/or CCL4 that is not increased or is decreased relative to the control sample indicates that the patient is not likely to respond to treatment or be resistant to treatment with the BTK inhibitor.

In some embodiments, methods are provided for predicting whether a patient will respond therapeutically to a method of treating DLBCL comprising administering ibrutinib, wherein the method comprises: (a) measuring in a sample (e.g., a serum sample) obtained from the patient the expression level of CCL3 and/or CCL4; (b) comparing the level obtained in step (a) to the level of expression of said CCL3 and/or CCL4 in a control sample, wherein an increase in the level of CCL3 and/or CCL4 measured in step (a) indicates that the patient will respond therapeutically to treatment with ibrutinib, whereas the level of CCL3 and/or CCL4 that is not increased or is decreased relative to the control sample indicates that the patient is not likely to respond to treatment or be resistant to treatment with ibrutinib.

In some embodiments, the methods provided herein are practiced iteratively over time, wherein decreased levels of CCL3 and/or CCL4 in the patient sample relative to a pretreatment or reference sample suggest a favorable response of a patient to treatment with a BTK inhibitor (e.g., ibrutinib) and levels of CCL3 and/or CCL4 in the patient sample that are increased relative to the control sample indicates that the patient is not likely to respond to treatment or be resistant to treatment with the BTK inhibitor. Accordingly, in some embodiments, methods are provided of monitoring the treatment of a patient having DLBCL, wherein the DLBCL is treated by a method comprising administering one or more BTK inhibitors to the patient. “One or more” BTK inhibitors include, for example, a single BTK inhibitor used alone or in combination with an anti-cancer agent or a neoplastic agent. In some embodiments, the BTK inhibitor is ibrutinib.

In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following treatment with a BTK inhibitor. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a single dosage with a BTK inhibitor. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a multiple administrations with a BTK inhibitor. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following a multiple administrations with a BTK inhibitor and after the last dosage. In some embodiments, level of CCL3 and/or CCL4 expression is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following administration of the last dosage a BTK inhibitor in a treatment regimen. In some embodiments, the BTK inhibitor is ibrutinib.

In some embodiments, level of CCL3 and/or CCL4 expression is monitored over time during the course of a treatment regimen with a BTK inhibitor. In some embodiments, level of CCL3 and/or CCL4 expression is measured every day, every 2 days, every 3 days, every 4 days, every 5 days, every 6 days, every week, every 2 weeks, every 3 weeks, every month or longer interval. In some embodiments, level of CCL3 and/or CCL4 expression is monitored over time during the course of a treatment regimen with a BTK inhibitor where the level of CCL3 and/or CCL4 expression is determined following each administration of the BTK inhibitor. In some embodiments, level of CCL3 and/or CCL4 expression is monitored over time during the course of a treatment regimen with a BTK inhibitor where the level of CCL3 and/or CCL4 expression is determined following multiple administrations of the BTK inhibitor. In some embodiments, level of CCL3 and/or CCL4 expression is monitored over time during the course of a treatment regimen with a BTK inhibitor where the level of CCL3 and/or CCL4 expression is determined following 2, 3, 4, 5, 6, 7, 8, 9, 10, 11, 12 or more administrations of the BTK inhibitor. In some embodiments, the BTK inhibitor is ibrutinib.

In some embodiments, the level of CCL3 and/or CCL4 expression is monitored over time during the course of a treatment regimen with a BTK inhibitor, wherein an increase in the level of CCL3 and/or CCL4 expression indicates that the patient is resistant or will become resistant to treatment with the BTK inhibitor. In some embodiments, the level of CCL3 and/or CCL4 expression is monitored over time during the course of a treatment regimen with a BTK inhibitor, wherein if a change in the level of CCL3 and/or CCL4 expression is detected relative to a reference level, the treatment regimen is modified, continued or discontinued. In some embodiments, the treatment regimen is discontinued. In some embodiments, the treatment regimen is continued. In some embodiments, the treatment regimen is modified. In some embodiments, the dosage of the BTK inhibitor is increased. In some embodiments, the dosage of the BTK inhibitor is decreased. In some embodiments, the dosage of the BTK inhibitor not modified. In some embodiments, the frequency of administration of the BTK inhibitor is increased. In some embodiments, the frequency of administration of the BTK inhibitor is decreased. In some embodiments, the frequency of administration of the BTK inhibitor is not modified. In some embodiments, the timing of administration of the BTK inhibitor is modified (e.g., time of day or time relative to administration of other therapeutic agents). In some embodiments, the timing of administration of the BTK inhibitor is not modified. In some embodiments, an additional therapeutic agent is administered. In some embodiments, the BTK inhibitor is ibrutinib.

In some embodiments, where the level of CCL3 and/or CCL4 expression is increased over the course of therapy with a BTK inhibitor, the dosage of the BTK inhibitor is increased. In some embodiments, where the level of CCL3 and/or CCL4 expression is increased over the course of therapy, the frequency of administration of the BTK inhibitor is increased. In some embodiments, where the level of CCL3 and/or CCL4 expression is increased over the course of therapy, an additional therapeutic agent is administered.

In some embodiments, where the level of CCL3 and/or CCL4 expression is increased over the course of therapy with ibrutinib, the dosage of ibrutinib is increased. In some embodiments, where the level of CCL3 and/or CCL4 expression is increased over the course of therapy, the frequency of administration of ibrutinib is increased. In some embodiments, where the level of CCL3 and/or CCL4 expression is increased over the course of therapy, an additional therapeutic agent is administered.

In some embodiments, “high expression levels” of CCL3 in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of CCL3 protein. In some embodiments, “high expression levels” of CCL4 in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of CCL4 protein. In some embodiments, “high expression levels” of CCL3 in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of nucleic acid (e.g., mRNA) encoding CCL3 (i.e., increased expression from the CCL3/SCYA3 gene). In some embodiments, “high expression levels” of CCL4 in a patient relative to normal means that the patient exhibits a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher increase in the expression of nucleic acid (e.g., mRNA) encoding CCL4 (i.e., increased expression from the CCL4/SCYA4 gene).

In some embodiments, the method comprises obtaining a sample (e.g., a serum sample) from the patient and measuring the expression level of CCL3 and/or CCL4 protein. Exemplary CCL3 proteins include, but are not limited to, the human CCL3 protein is set forth in SEQ ID NO: 2. Exemplary CCL4 proteins include, but are not limited to, the human CCL4 protein is set forth in SEQ ID NO: 4. In some embodiments, measuring the expression level of CCL3 and/or CCL4 protein comprises an immunoassay. In some embodiments, measuring the expression level of CCL3 and/or CCL4 protein comprises an ELISA. In some embodiments, measuring the expression level of CCL3 and/or CCL4 protein comprises detecting CCL3 and/or CCL4 protein with an antibody. In some embodiments, the antibody is labeled. In some embodiments, measuring the expression level of CCL3 and/or CCL4 protein comprises detecting CCL3 and/or CCL4 protein with a first antibody to form an antibody complex with CCL3 and/or CCL4 protein, and then detecting the antibody complex with a secondary antibody that binds to the first antibody. In some embodiments, the antibody is labeled.

In some embodiments, the method comprises obtaining a sample containing nucleic acid from the patient and measuring the expression level of the nucleic acid encoding CCL3 and/or CCL4. Exemplary nucleic acids encoding CCL3 include, but are not limited to, the nucleic acid set forth in SEQ ID NO: 1. Exemplary nucleic acids encoding CCL4 include, but are not limited to, the nucleic acid set forth in SEQ ID NO: 3. In some embodiments, the method comprises isolating or purifying mRNA from the sample. In some embodiments, the method comprises amplifying the mRNA transcripts, e.g., by RT-PCR. In some embodiments, a higher baseline level of CCL3 and/or CCL4 (as assessed by, e.g., determining the cycle number at which the fluorescence passes the set threshold level (“ct”) of CCL3 and/or CCL4 mRNA expression) indicates a higher likelihood that the cancer will be sensitive to treatment with the BTK inhibitor.

In some embodiments, the sample for use in the methods is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is a blood serum sample. In particular embodiments, the sample is a tumor biopsy sample. In particular embodiments, the sample is from a fluid or tissue that is part of, or associated with, the lymphatic system or circulatory system. In some embodiments, the sample is a blood sample that is a venous, arterial, peripheral, tissue, cord blood sample. In particular embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g. in a bone marrow aspirate sample).

In some embodiments, the samples are obtained from the subject by any suitable means of obtaining the sample using well-known and routine clinical methods. Procedures for obtaining fluid samples from a subject are well known. For example, procedures for drawing and processing whole blood and lymph are well-known and can be employed to obtain a sample for use in the methods provided. Typically, for collection of a blood sample, an anti-coagulation agent (e.g. EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the collection of a sample from the subject is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to treatment with one or more anti-cancer agents. In some embodiments, anticancer agent is administered for the treatment of a leukemia, lymphoma or a myeloma. Exemplary anti-cancer agents for the treatment of a leukemia, lymphoma or a myeloma include but are not limited to ibrutinib, adriamycin (doxorubicin), bexxar, bendamustine, bleomycin, blenoxane, bortezomib, dacarbazine, deltasone, cisplatin, cyclophosphamide, cytoxan, DTIC dacarbazine, dasatinib, doxorubicin, etoposide, fludarabine, granisetron, kytril, lenalidomide, matulane, mechlorethamine, mustargen, mustine, natulan, Rituxan (rituximab, anti-CD20 antibody), VCR, neosar, nitrogen mustard, oncovin, ondansetron, orasone, prednisone, procarbazine, thalidomide, VP-16, velban, velbe, velsar, VePesid, vinblastine, vincristine, Zevalin®, zofran, stem cell transplantation, radiation therapy or combination therapies, such as, for example, ABVD (adriamycin, bleomycin, vinblastine and dacarbazine), ChlvPP (chlorambucil, vinblastine, procarbazine and prednisolone), Stanford V (mustine, doxorubicin, vinblastine, vincristine, bleomycin, etoposide and steroids), BEACOPP (bleomycin, etoposide, doxorubicin, cyclophosphamide, vincristine, procarbazine and prednisolone), BEAM (carmustine (BiCNU) etoposide, cytarabine (Ara-C, cytosine arabinoside), and melphalan), CHOP (cyclophosphamide, doxorubicin, vincristine, and prednisone), R-CHOP (rituximab, doxorubicin, cyclophosphamide, vincristine, and prednisone), EPOCH (etoposide, vincristine, doxorubicin, cyclophosphamide, and prednisone), CVP (cyclophosphamide, vincristine, and prednisone), ICE (ifosfamide-carboplatin-etoposide), R-ACVBP (rituximab, doxorubicin, cyclophosphamide, vindesine, bleomycin, and prednisone), DHAP (dexamethasone, high-dose cytarabine, (Ara C), cisplatin), R-DHAP(rituximab, dexamethasone, high-dose cytarabine, (Ara C), cisplatin), ESHAP (etoposide (VP-16), methyl-prednisolone, and high-dose cytarabine (Ara-C), cisplatin), CDE (cyclophosphamide, doxorubicin and etoposide), Velcade® (bortezomib) plus Doxil® (liposomal doxorubicin), Revlimid® (lenalidomide) plus dexamethasone, and bortezomib plus dexamethasone. In some embodiments, anticancer agent is fludarabine. In some embodiments, anticancer agent is bendamustine. In some embodiments, the anticancer agent is Rituxan. In some embodiments, the anticancer agent is dasatinib. In some embodiments, a sample is collected at a predetermined time or at regular intervals prior to, during, or following treatment or between successive treatments with the anti-cancer agent. In particular examples, a sample is obtained from the subject prior to administration of an anti-cancer therapy and then again at regular intervals after treatment has been effected.

In some embodiments, the collection of a sample is performed at a predetermined time or at regular intervals relative to treatment with a BTK inhibitor. For example, a sample is collected from a patient at a predetermined time or at regular intervals prior to, during, or following treatment or between successive treatments with the BTK inhibitor. In particular examples, a sample is obtained from a patient prior to administration of a BTK inhibitor and then again at regular intervals after treatment with the BTK inhibitor has been effected. In some embodiments, the patient is administered a BTK inhibitor and one or more additional anti-cancer agents. In some embodiments, the BTK inhibitor is an irreversible BTK inhibitor. In some embodiments, the BTK inhibitor is a reversible BTK inhibitor. In some embodiments, the BTK inhibitor is ibrutinib. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), ACP-196 (Acerta), PRN1008 (Principia), CTP-730 (Concert Pharmaceuticals), and GDC-0853 (Genentech).

In some embodiments, the patient is administered a BTK inhibitor and one or more additional anti-cancer agents. In some embodiments, the patient is administered a BTK inhibitor and one or more additional anti-cancer agents that are not BTK inhibitors. In some embodiments, the patient is administered a BTK inhibitor and one or more additional anti-cancer agents that are BTK inhibitors. In some embodiments, the patient is administered ibrutinib and one or more additional anti-cancer agents that are BTK inhibitors. In some embodiments, the patient is administered ibrutinib and one or more additional anti-cancer agents that are not BTK inhibitors. In some embodiments, the one or more additional anti-cancer agents includes a reversible BTK inhibitor. In some embodiments, the one or more additional anti-cancer agents includes an irreversible BTK inhibitor. In some embodiments, the subject is administered one or more irreversible BTK inhibitors. In some embodiments, the subject is administered one or more reversible BTK inhibitors.

In some embodiments, the subject is administered ibrutinib in combination with one or more reversible BTK inhibitors. For example, in some embodiments, the subject is administered ibrutinib in combination with one or more reversible BTK inhibitors that are not dependent on cysteine 481 for binding. Reversible BTK inhibitors are known in the art and include, but are not limited to, dasatinib, PC-005, RN486, PCI-29732 or terreic acid. In a particular embodiment, the irreversible BTK inhibitor ibrutinib is administered in combination with the reversible BTK inhibitor dasatinib.

In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the irreversible BTK inhibitor. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to a subject naïve for exposure to ibrutinib. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of a BTK inhibitor to a subject having DLBCL. In some embodiments, the sample is obtained 1, 2, 3, 4, 5, 6, 7, 8, 9, 10 times or more over the course of treatment with a BTK inhibitor. In some embodiments, the subject is responsive the treatment with a BTK inhibitor when it is first administered.

In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the irreversible BTK inhibitor. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to a subject naïve for exposure to ibrutinib. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to a subject having DLBCL. In some embodiments, the sample is obtained 1, 2, 3, 4, 5, 6, 7, 8, 9, 10 times or more over the course of treatment with ibrutinib. In some embodiments, the subject is responsive the treatment with ibrutinib when it is first administered.

In some embodiments, the level of expression of CCL3 and/or CCL4 in a sample is compared to the level of expression in a reference DLBCL cell or population of DLBCL cells. In some embodiments, the level of expression of CCL3 and/or CCL4 in a sample is compared to the level of expression in a reference DLBCL cell line. In some embodiments, the level of expression of CCL3 and/or CCL4 in a sample is compared to the level of expression in a reference DLBCL cell or population of DLBCL cells that is known to be resistant to treatment with a BTK inhibitor. In some embodiments, the level of expression of CCL3 and/or CCL4 in a sample is compared to the level of expression in a reference DLBCL cell or population of DLBCL cells that is known to be sensitive to treatment with a BTK inhibitor. In some embodiments, the level of expression of CCL3 and/or CCL4 in a sample is compared to the level of expression in a reference DLBCL cell line that is known to be resistant to treatment with a BTK inhibitor. In some embodiments, the level of expression of CCL3 and/or CCL4 in a sample is compared to the level of expression in a reference DLBCL cell line that is known to be sensitive to treatment with a BTK inhibitor. In some embodiments, the DLBCL cell line is an activated B-cell-like (ABC)-DLBCL cell line. In some embodiments, the DLBCL cell line is a germinal center B-cell-like (GCB)-DLBCL cell line. In some embodiments, the DLBCL cell line is OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, or TMD8. In some embodiments, the DLBCL cell line sensitive to treatment with a BTK inhibitor is TMD8, HBL-1 or OCI-Ly10. In some embodiments, the DLBCL cell line resistant to treatment with a BTK inhibitor is OCI-Ly3, DB or OCI-Ly19.

### Maintenance Therapy

Provided herein are methods for maintenance therapy of subject having DLBCL. In some embodiments, the methods for maintenance therapy comprise treating DLBCL with a BTK inhibitor for a period of six months or longer, such as, for example, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 3 years, 4 years, 5 years, 6 years, 7 years, 8 years, 9 years, 10 years or longer. In some embodiments, the methods for maintenance therapy comprise treating DLBCL with ibrutinib for a period of six months or longer, such as, for example, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 3 years, 4 years, 5 years, 6 years, 7 years, 8 years, 9 years, 10 years or longer.

In an exemplary method, a subject having DLBCL is treated with a therapeutically effective amount of a BTK inhibitor for longer than six months and after about six months of treatment, the subject is monitored at predetermined intervals of time to determine level of expression of CCL3 and/or CCL4. In some embodiments, monitoring comprises: (a) determining an expression level of CCL3 and/or CCL4 in a sample (e.g., a serum sample) following or during the course of treatment with a BTK inhibitor; and (b) modifying or continuing the treatment based on the expression of CCL3 and/or CCL4 relative to a control or reference level. In some embodiments, a reference level is level of expression of CCL3 and/or CCL4 in a sample (e.g., a serum sample) taken from the patient prior to administration of the therapeutically effective amount of the BTK inhibitor. In some embodiments, a reference level is the level of expression of CCL3 and/or CCL4 in a normal patient (e.g., a patient without DLBCL). In some embodiments, the treatment regimen is continued. In some embodiments, the treatment regimen is modified. In some embodiments, the dosage of the BTK inhibitor is increased. In some embodiments, the dosage of the BTK inhibitor is decreased. In some embodiments, the dosage of the BTK inhibitor not modified. In some embodiments, the frequency of administration of the BTK inhibitor is increased. In some embodiments, the frequency of administration of the BTK inhibitor is decreased. In some embodiments, the frequency of administration of the BTK inhibitor is not modified. In some embodiments, the timing of administration of the BTK inhibitor is modified (e.g., time of day or time relative to administration of other therapeutic agents). In some embodiments, the timing of administration of the BTK inhibitor is not modified. In some embodiments, an additional therapeutic agent is administered. In some embodiments, an additional anti-cancer agent is administered.

In some embodiments, the method further comprises discontinuing treatment with a BTK inhibitor if level of expression of CCL3 and/or CCL4 increase relative to a reference or control over the course of treatment with the BTK inhibitor. In some embodiments, the method further comprises continuing treatment with a BTK inhibitor if level of expression of CCL3 and/or CCL4 decreases relative to a reference level. In some embodiments, the reference is level of expression of CCL3 and/or CCL4 in a DLBCL patient. In some embodiments, the reference is level of expression of CCL3 and/or CCL4 in the same patient prior to treatment with a BTK inhibitor. In some embodiments, the method further comprises administering an inhibitor of LYN, SYK, JAK, PI3K, PLCy, MAPK, or MEK. In some embodiments, the method further comprises administering an additional inhibitor of BTK. In some embodiments, the method further comprises administering a reversible inhibitor of BTK. In some embodiments, the method further comprises administering an irreversible inhibitor of BTK.

In some embodiments, the method further comprises discontinuing treatment with ibrutinib if level of expression of CCL3 and/or CCL4 increase relative to a reference or control over the course of treatment with ibrutinib. In some embodiments, the method further comprises continuing treatment with ibrutinib if level of expression of CCL3 and/or CCL4 decreases relative to a reference level. In some embodiments, the reference is level of expression of CCL3 and/or CCL4 in a DLBCL patient. In some embodiments, the reference is level of expression of CCL3 and/or CCL4 in the same patient prior to treatment with ibrutinib. In some embodiments, the method further comprises administering an inhibitor of LYN, SYK, JAK, PI3K, PLCy, MAPK, or MEK. In some embodiments, the method further comprises administering a reversible inhibitor of BTK. In some embodiments, the method further comprises administering an additional reversible inhibitor of BTK.

In some embodiments, the subject is monitored every month, every 2 months, every 3 months, every 4 months, every 5 months, every 6 months, every 7 months, every 8 months, every 9 months, every 10 months, every 11 months, or every year to determine the level of expression of CCL3 and/or CCL4.

In some embodiments, maintenance therapy comprises multiple cycles of administration of a BTK inhibitor. In some embodiments, a cycle of administration is one month, 2 months, 3 months, 4 months, 6 months, 6 months, 7 months, 8 months, 9 months, 10 months, 11 months, 12 months or longer. In some embodiments, a cycle of administration comprises administration of a single therapeutic dosage of a BTK inhibitor over the cycle. In some embodiments, a cycle of administration comprises two or more different dosages of a BTK inhibitor over the cycle. In some embodiments, the dosage of a BTK inhibitor differs over consecutive cycles. In some embodiments, the dosage of a BTK inhibitor increases over consecutive cycles. In some embodiments, the dosage of a BTK inhibitor is the same over consecutive cycles.

In some embodiments, maintenance therapy comprises multiple cycles of administration of ibrutinib. In some embodiments, a cycle of administration is one month, 2 months, 3 months, 4 months, 6 months, 6 months, 7 months, 8 months, 9 months, 10 months, 11 months, 12 months or longer. In some embodiments, a cycle of administration comprises administration of a single therapeutic dosage of ibrutinib over the cycle. In some embodiments, a cycle of administration comprises two or more different dosages of ibrutinib over the cycle. In some embodiments, the dosage of ibrutinib differs over consecutive cycles. In some embodiments, the dosage of ibrutinib increases over consecutive cycles. In some embodiments, the dosage of ibrutinib is the same over consecutive cycles.

In some embodiments, maintenance therapy comprises administration of a daily dosage of a BTK inhibitor. In some embodiments, the daily dosage of ibrutinib administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the maintenance dosage of a BTK inhibitor is about 840 mg per day. In a particular embodiment, the maintenance dosage of a BTK inhibitor is about 560 mg per day. In a particular embodiment, the maintenance dosage of a BTK inhibitor is about 420 mg per day. In a particular embodiment, the maintenance dosage of a BTK inhibitor is about 140 mg per day.

In some embodiments, maintenance therapy comprises administration of a daily dosage of ibrutinib. In some embodiments, the daily dosage of ibrutinib administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the maintenance dosage of ibrutinib is about 840 mg per day. In a particular embodiment, the maintenance dosage of ibrutinib is about 560 mg per day. In a particular embodiment, the maintenance dosage of ibrutinib is about 420 mg per day. In a particular embodiment, the maintenance dosage of ibrutinib is about 140 mg per day.

In some embodiments, a BTK inhibitor is administered once per day, two times per day, three times per day or more frequent. In a particular embodiment, a BTK inhibitor is administered once per day.

In some embodiments, ibrutinib is administered once per day, two times per day, three times per day or more frequent. In a particular embodiment, ibrutinib is administered once per day.

In some embodiments, the dosage of a BTK inhibitor is escalated over time. In some embodiments, the dosage of a BTK inhibitor is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, the dosage of ibrutinib is escalated over time. In some embodiments, the dosage of ibrutinib is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, a cycle of administration comprises administration of a BTK inhibitor in combination with an additional therapeutic agent. In some embodiments the additional therapeutic is administered simultaneously, sequentially, or intermittently with a BTK inhibitor. In some embodiments the additional therapeutic agent is an anti-cancer agent. In some embodiments the additional therapeutic agent is an anti-cancer agent for the treatment of a leukemia, lymphoma or a myeloma. Exemplary anti-cancer agents for administration in a combination with a BTK inhibitor are provided elsewhere herein. In a particular embodiment, the anti-cancer agent is an anti-CD 20 antibody (e.g. Rituxan). In a particular embodiment, the anti-cancer agent bendamustine. In some embodiments, the additional anti-cancer agent is a reversible BTK inhibitor.

In some embodiments, a cycle of administration comprises administration of ibrutinib in combination with an additional therapeutic agent. In some embodiments the additional therapeutic is administered simultaneously, sequentially, or intermittently with ibrutinib. In some embodiments the additional therapeutic agent is an anti-cancer agent. In some embodiments the additional therapeutic agent is an anti-cancer agent for the treatment of a leukemia, lymphoma or a myeloma. Exemplary anti-cancer agents for administration in a combination with ibrutinib are provided elsewhere herein. In a particular embodiment, the anti-cancer agent is an anti-CD 20 antibody (e.g. Rituxan). In a particular embodiment, the anti-cancer agent bendamustine. In some embodiments, the additional anti-cancer agent is a reversible BTK inhibitor.

In some embodiments, provided is a method for monitoring whether a subject receiving maintenance therapy with a BTK inhibitor for treatment of DLBCL has developed or is likely to develop resistance to the therapy, comprising determining the expression level of CCL3 and/or CCL4 over the course of therapy.

In some embodiments, the method further comprises discontinuing treatment with a BTK inhibitor if the level of the expression of CCL3 and/or CCL4 increases over the course of therapy. In some embodiments, an increase in the level of the expression of CCL3 and/or CCL4 over the course of the therapy indicates that the patient is resistant to the therapy or that the patient will become resistant to the therapy. In some embodiments, the method further comprises continuing treatment with ibrutinib if the level of the expression of CCL3 and/or CCL4 decreases over the course of therapy. In some embodiments, the method further comprises administering an inhibitor of LYN, SYK, JAK, PI3K, PLCy, MAPK, or MEK. In some embodiments, the method further comprises administering an additional inhibitor of BTK. In some embodiments, the method further comprises administering a reversible inhibitor of BTK. In some embodiments, the method further comprises administering an irreversible inhibitor of BTK.

In some embodiments, provided is a method for optimizing the therapy of a subject receiving maintenance therapy with a BTK inhibitor for treatment of DLBCL, comprising: determining the expression level of CCL3 and/or CCL4 over the course of therapy. In some embodiments, the method further comprises continuing, discontinuing, or modifying treatment based on the expression level of CCL3 and/or CCL4. In some embodiments, the method further comprises administering an inhibitor of LYN, SYK, JAK, PI3K, PLCγ, MAPK, or MEK. In some embodiments, the method further comprises administering an additional inhibitor of BTK. In some embodiments, the method further comprises administering a reversible inhibitor of BTK. In some embodiments, the method further comprises administering an irreversible inhibitor of BTK.

### Kits and Articles of Manufacture

For use in the diagnostic and therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from any acceptable material including, e.g., glass or plastic.

In some embodiments, the kits provided herein are for use in determining the level of expression of CCL3 and/or CCL4.

In some embodiments, the kits provided herein are for use as a companion diagnostic with a BTK inhibitor. In some embodiments the kits are employed for selecting patients for treatment with a BTK inhibitor, for identifying subjects as sensitive to a BTK inhibitor of for evaluating treatment with a BTK inhibitor. In some embodiments the kits are employed for selecting patients for treatment with a BTK inhibitor, for identifying subjects as resistant or likely to become resistant to a BTK inhibitor, for monitoring the development of resistance to a BTK inhibitor, or combinations thereof.

In some embodiments, the kits provided herein are for use as a companion diagnostic with ibrutinib. In some embodiments the kits are employed for selecting patients for treatment with ibrutinib, for identifying subjects as sensitive to ibrutinib of for evaluating treatment with ibrutinib. In some embodiments the kits are employed for selecting patients for treatment with ibrutinib, for identifying subjects as resistant or likely to become resistant to ibrutinib, for monitoring the development of resistance to ibrutinib, or combinations thereof.

The kits provided herein contain one or more reagents for the detection of CCL3 and/or CCL4 expression. Exemplary reagents include but are not limited to, antibodies, buffers, nucleic acids, microarrays, ELISA plates, substrates for enzymatic staining, chromagens or other materials, such as slides, containers, microtiter plates, and optionally, instructions for performing the methods. Those of skill in the art will recognize many other possible containers and plates and reagents that can be used for contacting the various materials.

### EXAMPLES

These examples are provided for illustrative purposes only and not to limit the scope of the claims provided herein.

### Example 1

### Materials

Ibrutinib (PCI-32765, Pharmacyclics, Inc.) was stored as 10 mM stock solution in 100% DMSO at −20° C. This stock solution was diluted in complete RPMI medium with 10% FBS, L-glutamine (HyClone Laboratories, Logan, Utah) and penicillin-streptomycin (Cellgro, Hemdon, Va.) and was added to the assay medium to a final concentration of 1 μM or to the indicated ibrutinib concentration. For BCR stimulation, culture medium was supplemented with 10 μg/mL anti-IgM (polyclonal goat F(ab′)₂ fragments to human IgM, MP Biomedicals) for the indicated time periods.

### Example 2

### DLBCL Cell Lines

All DLBCL cell lines were grown in RPMI 1640 medium (Hyclone) supplemented with glutamine, beta-mercaptoethanol, penicillin/streptomycin and 10% fetal bovine serum, except for OCI-Ly3 and OCI-Ly10, which were maintained in Iscove's modified Dulbecco's medium (Gibco) supplemented with beta-mercaptoethanol, penicillin/streptomycin and 20% heparinized human plasma. All cell lines were grown in a humidified 5% CO₂ incubator at 37° C. DLBCL cell lines included TMD8, HBL-1, OCI-Ly10, OCI-Ly3, DB and OCI-Ly19.

### Example 3

### Quantification of Cellular CCL3 and CCL4 Levels

DLBCL cells were stimulated with anti-IgM (10 μg/mL) alone or in combination with ibrutinib (1 μM/mL) treatment. Cell lines HBL-1 and TMD8 were additionally titrated for different anti-IgM concentrations (0.1-10 μg) and different ibrutinib concentrations (3.1-1000 nM). Cell culture supernatant samples from DLBCL cell lines were used after 24 h for the measurement of CCL3 and CCL4 levels for quantification by ELISA using Quantikine Kits (R&D Systems). The absorbance was recorded by a microplate reader (ELx808, Bio-Tek Instruments), and data collection and analysis were performed using Gen5 software Version 1.08 (Bio-Tek Instruments).

### Example 4

### CCL3 and CCL4 Levels after BCR Activation Followed by Ibrutinib Treatment in DLBCL Cell Lines

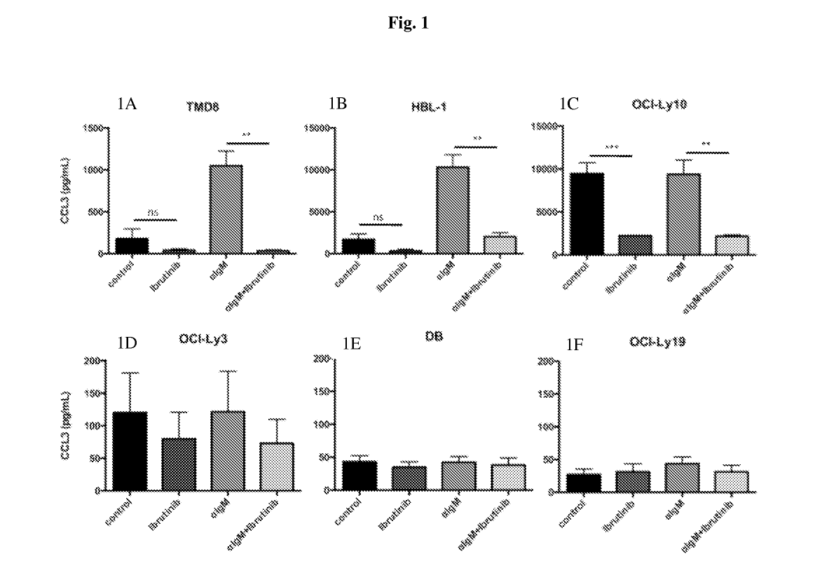

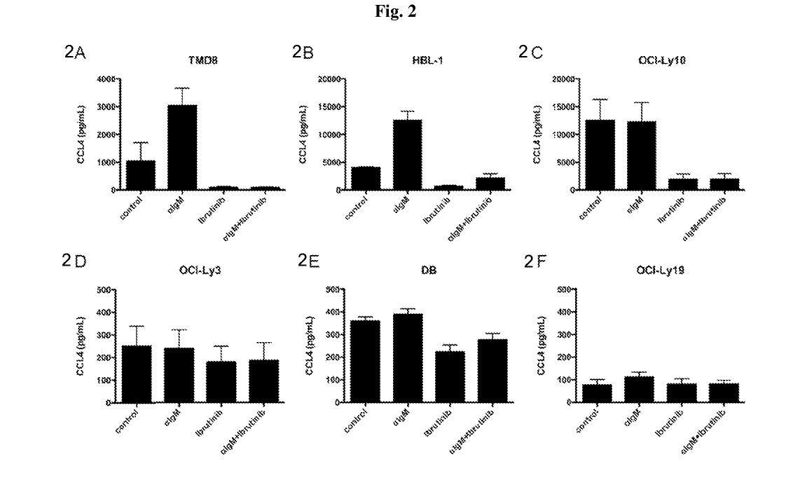

BCR signaling plays a central role for cell survival in the activated B cell-like (ABC) subtype of DLBCL. Furthermore the ABC DLBCL subtype relies on constitutive NF-kB signaling to block apoptosis whereas the germinal center B cell-like (GCB) subtype does not. The CCL3/4 secretion after anti-IgM stimulation and ibrutinib treatment were measured in a total of six cell lines, four of which were ABC subtype cell lines (TMD8, HBL-1, OCI-Ly10 and OCI-Ly3) and two were GCB cell lines (DB and OCI-Ly19). Anti-IgM-induced BCR signaling triggered secretion of high concentrations of CCL3 and CCL4 by TMD8 and HBL-1, which was abrogated by ibrutinib (FIG. 1 and FIG. 2). In contrast, OCI-Ly10 cells secreted high concentrations of CCL3 and CCL4 without stimulation, and there was no further increase in CCL3 or CCL4 concentrations by stimulation with anti-IgM. Nonetheless, CCL3 and CCL4 secretion by OCI-Ly10 cells was sensitive to inhibition by ibrutinib (FIGS. 1C and 2C). OCI-Ly3 cells, as well as the tested GCB cell lines did not secrete high CCL3 or CCL4 concentrations and did not respond to BCR stimulation or ibrutinib treatment (FIG. 1D-1F and FIG. 2D-2F). Specifically, TMD8 and HBL-1 cells have mutations in CD79B, while OCI-Ly10 has a CD79A mutation; both of these genes are essential for transmission of BCR signals, and the mutations found contribute to the chronic active BCR signaling in these cell lines. In contrast, OCI-Ly3 cells do not display chronic active BCR signaling, although they have an ABC DLBCL phenotype, which is attributed to a gain-of-function mutation in CARD11, a downstream mediator of the effects of BCR signaling that is wild-type in the TMD8 and HBL-1 ABC DLBCL lines. Treatment with ibrutinib significantly decreased CCL3 (FIG. 1) and CCL4 (FIG. 2) levels in ABC DLBCL cells with wild type CARD11. For example, in HBL-1 cells, anti-IgM stimulation significantly increased baseline CCL3 concentrations from 1705.5 (±27.5) pg/mL to 10324.3 (±125.3) pg/mL. Ibrutinib treatment of unstimulated cells decreased baseline CCL3 concentrations to 313.2 (±6.8) pg/mL and reduced anti-IgM-induced CCL3 to 2029.16 (±26.9) pg/mL (FIG. 1B).

### Example 5

### Sensitivity of the HBL-1 and TMD8 ABC DLBCL Cell Lines to BCR Stimulation and Ibrutinib Treatment

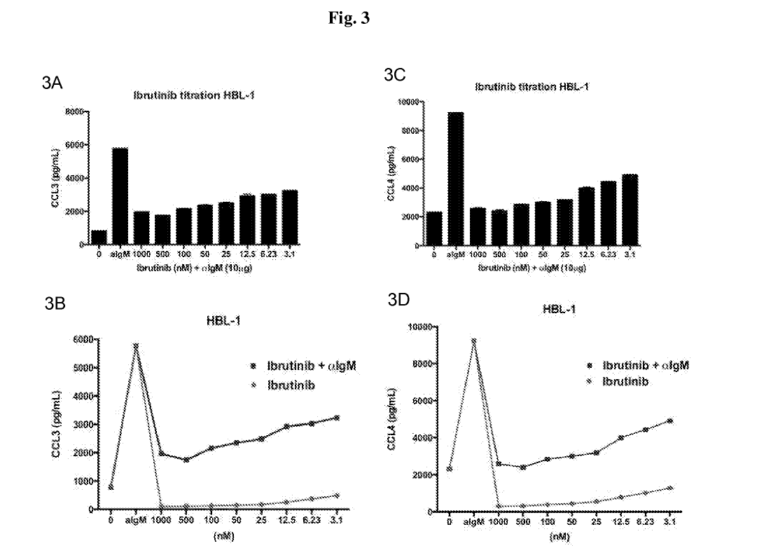

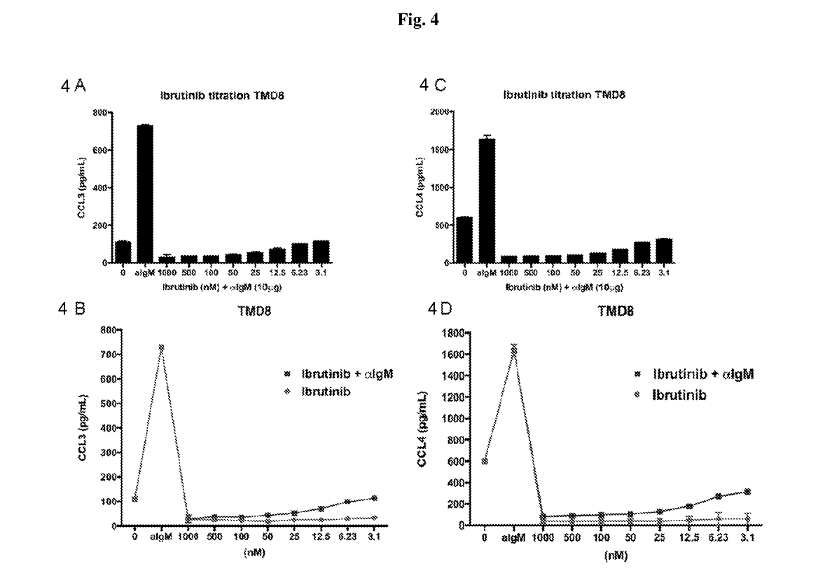

Various concentrations of ibrutinib were titrated in ABC cell lines HBL-1 (FIG. 3) and TMD8 (FIG. 4). In both cell lines, ibrutinib was either titrated in combination with 10 μg αIgM or alone. In the HBL-1 cell line after BCR stimulation, treatment with ibrutinib at 500 nM concentration in combination with 10 μg αIgM resulted in the lowest CCL3 level (FIGS. 3A and 3B) and CCL4 level (FIGS. 3C and 3D). In the TMD8 cell line after BCR stimulation, the CCL3 level was decreased at 1000 nM concentration of ibrutinib treatment in combination with 10 μg αIgM and this decreased level was maintained at 500 nM and 100 nM concentration of ibrutinib treatments (FIG. 4A-4B). Similarly, the CCL4 level was decreased at 1000 nM concentration of ibrutinib treatment in combination with 10 μg αIgM and this decreased level was maintained at 500 nM, 100 nM and 50 nM concentrations of ibrutinib treatments (FIG. 4C-4D).

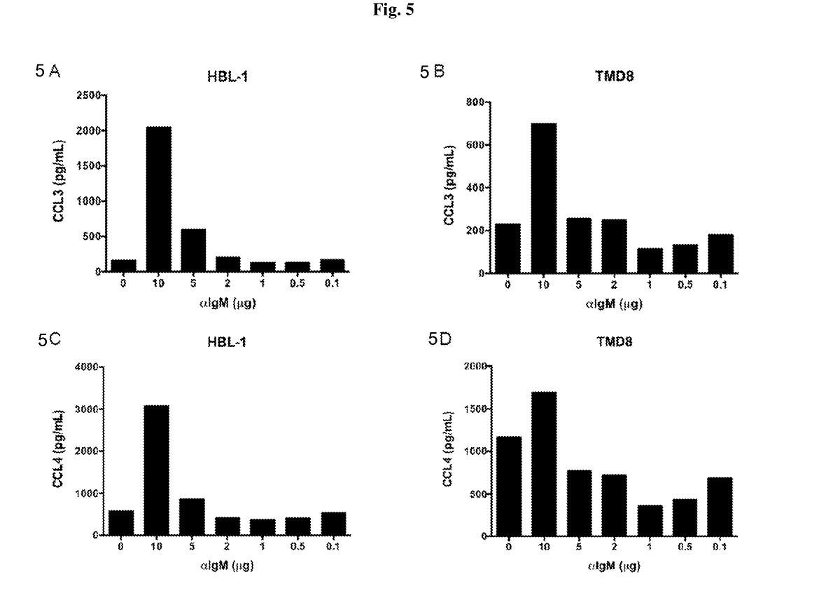

Both ABC cell lines HBL-1 and TMD8 were also responsive to stimulation of different αIgM concentrations. A significant increase in CCL3 and CCL4 levels in HBL-1 (FIG. 5A-5B) and TMD8 (FIG. 5C-5D) cell lines occurred at 10 μg/mL of αIgM.

### Example 6

### Serum CCL3 and CCL4 Levels as Prognostic Marker in DLBCL

Diffuse large B cell lymphoma (DLBCL) is a heterogeneous group of diseases, and can be demonstrated by several clinical and molecularly defined prognostic models. For example, the International Prognostic Index (IPI) and its revised version (R-IPI) have been widely used as a predictive model in clinics for risk-adaptive therapy in DLBCL. In addition, several gene expression profiling (GEP) models have been tested to predict outcome in patients with DLBCL. One of these models uses cDNA microarray to distinguish DLBCL into two prognostically distinct subtypes: activated B cell-like (ABC) phenotype and germinal center B cell-like (GCB) phenotype. Multivariate and integrative analysis of representative genes from these GEP models and other studies reduced the number of genes necessary for prediction to only six genes. These six genes can predict the outcome of patients with DLBCL independent of IPI.

B cell receptor (BCR) signaling is a critical growth and survival pathway in various B cell malignancies, including DLBCL. Upon BCR stimulation, normal and malignant B cells secrete chemokines CCL3 and CCL4 (MIP-1 α and β) to foster B cell interactions with accessory cells such as T helper cells. Previous studies have reported that plasma CCL3 and CCL4 levels were elevated in patients with chronic lymphocytic leukemia (CLL), and that CCL3 and CCL4 can function as robust clinical prognostic marker in CLL. Moreover, elevated CCL3 and CCL4 levels rapidly normalize in CLL patients after pharmacologic inhibition of BCR signaling with the PI3 kinase delta inhibitor GS-1101/CAL-101/idelalisib or the BTK-inhibitor PCI-32765/ibrutinib/imbruvica.

The gene (SCYA3) encoding CCL3 is one of the six genes that can predict the outcome of patients with DLBCL. It is overexpressed in ABC phenotype and the overexpression of SCYA3 alone is a strong prognosis in univariate analysis.

As provided herein, the serum levels of CCL3 and CCL4 were investigated in the ABC and GCB subtypes of DLBCL and were correlated with DLBCL prognosis. The data demonstrated that high serum CCL3 level correlated with poor prognostic features in DLBCL and was associated with worse outcome in DLBCL. Additionally, CCL3 and CCL4 secretion in DLBCL cell lines were investigated in both DLBCL subtypes after IgM stimulation and treatment with ibrutinib. The data demonstrated a correlation between anti-IgM (αIgM) responsiveness in the ABC DLBL subtype in comparison with the GCB subtype, as well as sensitivity to ibrutinib treatment.

Methods

Patients

The study included 102 patients from MD Anderson Cancer Center (MDACC) with newly diagnosed DLBCL between January 2009 and February 2011 (MDA test cohort). In addition, a second set of 51 patients from MDACC with newly diagnosed DLBCL was included for validation analysis (MDA validation cohort). Nineteen patients with newly diagnosed DLBCL from the University of Nebraska were also included in the correlation analysis between serum CCL3/CCL4 levels and GCB vs. non-GCB subtype (Nebraska cohort). The Nebraska cohort did not have clinical annotation other than DLBCL subtype information. Demographics and other clinical parameters for both MDACC test and validation cohort were obtained at the time of initial referral to MDACC. DLBCL diagnosis was established by institutional pathologists according to the WHO classification. Immunophenotypic classification (GCB vs. non-GCB subtype) of DLBCL was determined by Hans criteria. The protocol for specimen handling and analyses followed the guidelines of the Declaration of Helsinki and was approved by the Institutional Review Board (IRB) at MDACC and the University of Nebraska Medical Center without releasing any subject's identifying information.

Measurement of Serum and Cell Culture Supernatant CCL3 and CCL4 Levels

Peripheral blood serum samples were obtained from the tissue bank of the Department of Lymphoma and Myeloma at MDACC or from the University of Nebraska Medical Center. All serum samples were aliquoted and stored at the time of initial referral to each institution. Serum and cell culture supernatant CCL3 and CCL4 levels were quantified by ELISA using Quantikine Kits (R&D Systems) and was conducted at MDACC (Sivina M, Hartmann E, Kipps T J, et al. “CCL3 (MIP-1alpha) plasma levels and the risk for disease progression in chronic lymphocytic leukemia.” Blood. 2011; 117(5):1662-1669.). The absorbance was recorded on a microplate reader (ELx808, Bio-Tek Instruments), and data collection and analysis were performed using Gen5 software Version 1.08 (Bio-Tek Instruments).

Statistical Analysis

Differences among variables were evaluated by the chi-square test and Mann-Whitney U test for categorical and continuous variables, respectively. Receiver operating curve (ROC) analysis was conducted to evaluate correlation between serum CCL3/CCL4 levels and immunophenotypic signatures by Hans criteria. Progression-free survival (PFS) was defined as the time interval between date of initial treatment and date of disease progression or death, whichever occurred first. Disease progression was defined as radiologically or biopsy confirmed relapse or progression of DLBCL after or during initial therapies. Overall survival (OS) was defined as the time interval between date of initial treatment and date of death or last follow-up date. Survival data was plotted according to the Kaplan-Meier method and group comparison was made by log-rank test. Multivariate Cox proportional hazards regression models were fitted to adjust the prognostic effect of each covariate. In all analysis, P<0.05 was considered as statistically significant. All statistical analyses were conducted by IBM SPSS ver. 21.0 (IBM Corp, Armonk, N.Y.).

Results

DLBCL Patient Characteristics

Baseline demographics and clinical characteristics of the 102 patients from MDA test cohort were analyzed. The median age was 58.5 years (range: 22 to 86) and 41% was female. Thirty nine (39%) patients had an Ann Arbor Stage I or II, while 63 (63%) patients had stage III or IV disease. Sixty five patients (64%) had low International Prognostic Index (IPI) scores (0-2) and 37 (36%) had high IPI (3-5). Mean and median levels of serum lactated dehydrogenase (LDH), β2 microglobulin (β2MG) and absolute lymphocytes count (ALC) were also analyzed. Based on the Hans criteria, 55 patients (54%) were classified as the GCB subtype and 19 patients (19%) were classified as the non-GCB subtype. Subtype classification was unknown in 28 (28%) patients. A majority of the patients (93%) received rituximab plus CHOP or CHOP-like chemotherapy regimen as frontline treatment and 7 patients underwent high dose chemotherapy followed by autologous stem cell transplant (auto-SCT) as a salvage regimen upon progression or relapse of the disease.

Serum CCL3 and CCL4 Levels and Correlation with Other Established Prognostic Factors in DLBCL

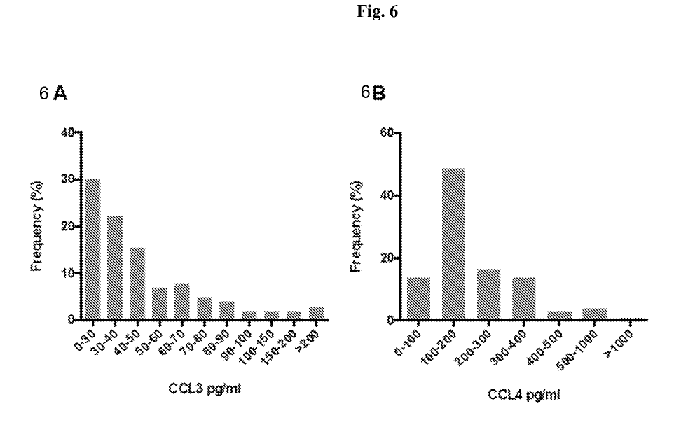

In the MDA test cohort, median (range) and mean (±SE, standard error) serum CCL3 levels were 39.2 (20.5-224.7) and 49.9 (±3.63) pg/ml respectively, while median and mean serum CCL4 levels were 178.5 (56.9-1173.1) and 224 (±17.4) pg/ml, respectively. Distribution of serum CCL3 levels in the MDA cohort was from 0-250 pg/ml and the distribution of CCL4 levels in the MDA cohort was from 0-1200 pg/ml (FIGS. 6A and 6B). The levels of CCL3 and CCL4 were dichotomized into high (or “not normal”) or low (or “normal”) based on the approximate median levels of CCL3 and CCL4 (40 pg/ml and 180 pg/ml, respectively), for serum levels above or below the median level (or cut-off level), respectively. High serum CCL3 level (≧40 pg/ml) was associated with higher IPI score (P=0.03), higher LDH (P=0.001), higher 132 microglobulin (P<0.001) and a strong trend with lower absolute lymphocyte count (P=0.006). High serum CCL4 level (≧180 pg/ml) was associated with advanced Ann Arbor stages (P<0.001) and a strong trend with higher IPI score (P=0.06). Additionally, high CCL3 and high CCL4 levels were correlated with each other (P=0.003).

Correlation Between CCL3/CCL4 Serum Concentrations and DLBCL Subtypes

Since the MDA test cohort had significant numbers of patients with missing information for Hans classification, 19 patients from the Nebraska cohort were added to evaluate correlation between serum CCL3/CCL4 levels and Hans classification. A total of 93 patients (74 from the MDA and 19 from the Nebraska cohort) were evaluated for immunophenotypic subtypes based on Hans criteria (GCB vs. non-GCB). Mean serum levels of CCL3 or CCL4 were not statistically different between GCB and non-GCB signatures (GCB vs. non-GCB: CCL3=33.7±2.5 pg/ml vs. 35.5±7.0 pg/mL, CCL4=207.1±21.5 pg/mL vs. 166.4±21.4 pg/mL).

Prognostic Impact of Serum CCL3/CCL4 Concentrations in DLBCL

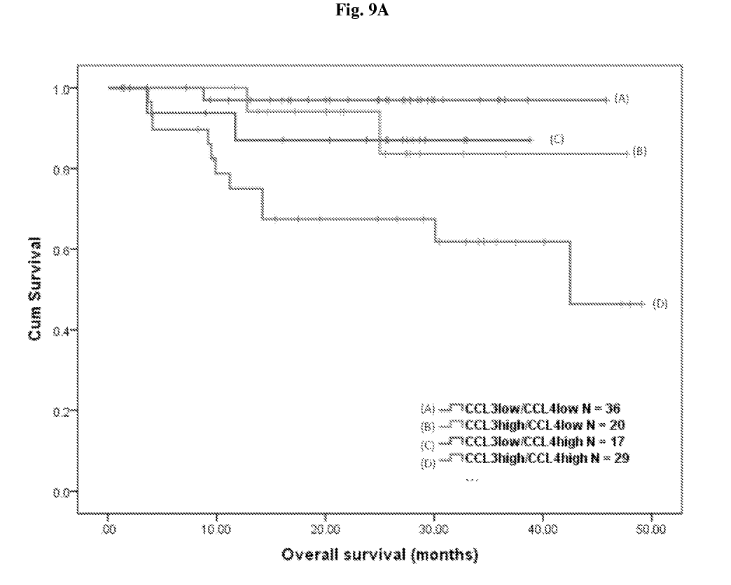

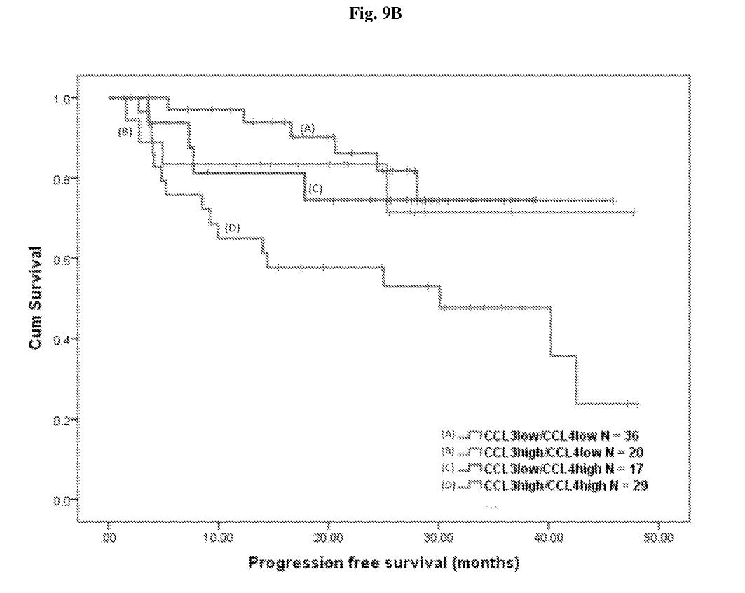

During the median follow up duration of 27.1 months (95% CI: 25.5-28.7 months), 16 patients (16%) died and 30 patients (29%) experienced progression of disease or death in the MDA test cohort (N=102). The MDA test cohort did not reach the median overall survival (OS) during the follow up duration, while median PFS was 42.5 months (95% CI; 27.7-52.3 months). Two year overall survival (OS) of the MDA test cohort was 77%, and 2 year progression free survival (PFS) was 56%. Log-rank test demonstrated that high serum CCL3 (≧40 pg/ml) level was associated with shorter OS (P=0.011) and PFS (P=0.031). Similarly, high serum CCL4 (≧180 pg/ml) level was also associated with shorter OS (P=0.015) and PFS (P=0.035). When both levels were considered, CCL3_{high}/CCL4_{high} group (N=29) had the worst PFS and OS by log-rank test (P=0.002 and P=0.008, both vs. CCL3_{low}/CCL4_{low} group) (FIGS. 9A and 9B).

The univariate log-rank test was also applied to other potential prognostic factors for OS and PFS in the MDA test cohort. Because serum CCL3 and CCL4 levels were strongly correlated with IPI score, the prognostic effect of serum CCL3 and CCL4 were adjusted with IPI score by multivariate Cox proportional hazards regression models. After adjusting the prognostic effect from IPI score, serum CCL3 levels had statistically significant prognostic impact in OS in this study.

Findings in a Validation Cohort

Serum CCL3 and CCL4 levels in DLBCL was evaluated in independent validation cohort (the MDA validation cohort, N=51). Demographics and clinical characteristics of the validation cohort were similar to that of the test cohort. Correlation to other prognostic markers in DLBCL confirmed the similar pattern of correlation demonstrated in the test cohort. Due to the short follow up duration and low event rate in the validation cohort, (median follow up 19.7 months, 95% CI: 18.0-21.4 months), prognostic association with OS and PFS in this study was not determinable.

Effect of Treatment on CCL3 and CCL4 Serum Concentrations

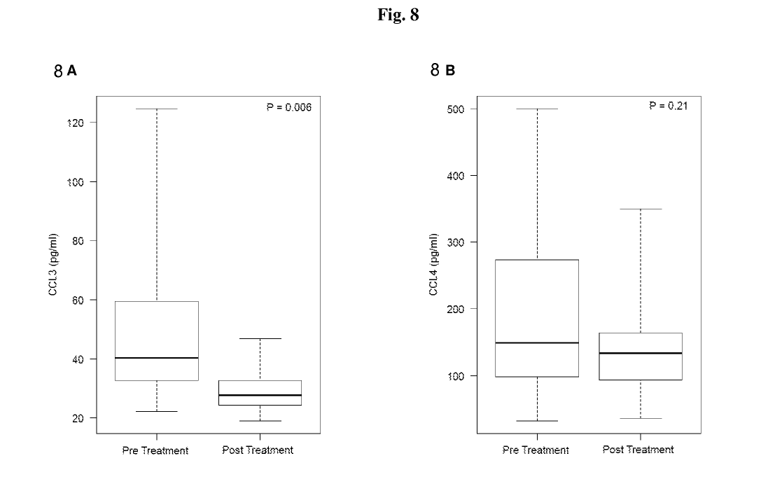

Samples were taken in nineteen patients at both pre- and post-treatment time points. The median time between pre- and post-treatment was 7.1 months (range: 4.1-18.4 months). At the time of the second measurement, 17 patients were in complete remission (CR) and 2 patients had partial response (PR) to treatment. Median level of pre- and post-treatment serum CCL3 levels were 40.3 pg/ml (range: 22.1-124.7) and 27.7 pg/ml (range: 18.9-46.8), respectively. Median level of pre- and post-treatment serum CCL4 levels were 149.4 pg/ml (range: 31.9-500.0) and 133.5 pg/ml (range: 36.3-349.4), respectively. The median change in serum CCL3 and CCL4 levels from pre-treatment to post-treatment were -13.7 pg/ml (range: −96.9-+7.9) and −20.6 pg/ml (range: −163.7-+4.4), respectively. Serum CCL3 level decreased in 15 out of 18 patients and serum CCL4 level decreased in 15 out of 18 patients (FIGS. 8A and 8B).

The same cut-off values (i.e., 40 pg/ml for CCL3 and 180 pg/ml for CCL4) were applied in additional analysis as a “normal limit” for CCL3 (i.e., <40 pg/ml) and CCL4 (i.e., <180 pg/ml). Serum CCL3 level was “normalized” in 15 patients, while serum CCL4 level was “normalized” in 14 patients. Within the 3 patients who did not achieve “normal” CCL3 level post-treatment, 1 patient experienced relapse of DLBCL at a later time. All 15 patients who achieved “normal” CCL3 level maintained their CR or PR without progression of the disease.

The examples and embodiments described herein are for illustrative purposes only and various modifications or changes suggested to persons skilled in the art are to be included within the spirit and purview of this application and scope of the appended claims.

What is claimed is:

1. A method for determining whether a patient diagnosed with diffuse large B cell lymphoma (DLBCL) is likely to respond to therapy with ibrutinib comprising:
a) determining a pre-administration expression level of CCL3 and/or CCL4 in a sample from the patient;
b) administering a dose of ibrutinib;
c) detecting a post-administration expression level of CCL3 and/or CCL4 in a sample from the patient following administration of ibrutinib to the patient; and
d) characterizing the patient as likely to respond to therapy with ibrutinib if the post-administration expression level of CCL3 and/or CCL4 decreased compared to the pre-administration level of CCL3 and/or CCL4.

2. The method of claim 1, wherein the dose of ibrutinib is about 140 mg to about 840 mg.

3. The method of claim 1, wherein the level of CCL3 and/or a CCL4 expression decreases by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of ibrutinib.

4. The method of claim 1, wherein the post-administration level of CCL3 and/or CCL4 decreases to the expression level of CCL3 and/or CCL4 in a human that does not have DLBCL.

5. The method of claim 1, wherein the post-administration expression level of CCL3 and/or CCL4 is measured 1 hour, 2 hour, 3 hours, 4 hours, 5 hour, 6 hours, 7 hours, 8 hours, 9 hours, 10 hours, 11 hours, 12 hours, 14 hours, 16 hours, 18 hours, 20 hours, 24 hours, 36 hours, 48 hours, or longer following administration of ibrutinib.

6. The method of claim 3, further comprising administering a therapeutically effective amount of ibrutinib to the patient.

7. The method of claim 1, wherein the sample is a blood sample or a serum sample.

8. The method of claim 1, wherein determining an expression level of CCL3 and/or CCL4 in the sample comprises measuring the amount of CCL3 and/or CCL4 proteins in the sample.

9. The method of claim 8, wherein the measuring the amount of CCL3 and/or CCL4 protein is with an enzyme-linked immunosorbent assay (ELISA).

10. The method of claim 1, wherein the DLBCL is activated B cell-like (ABC) subtype of DLBCL.

11. A method for treating DLBCL in a patient in need thereof comprising:
(a) determining a pre-administration expression level of CCL3 and/or CCL4 in a sample from the patient prior to administration of ibrutinib; and
(b) administering to the patient a therapeutically effective amount of ibrutinib if the pre-administration expression of CCL3 and/or CCL4 is increased relative to a control or reference level.

12. The method of claim 11, wherein the sample is a blood sample or a serum sample.

13. The method of claim 11, wherein the determining an expression level of CCL3 and/or CCL4 in the sample comprises measuring the amount of CCL3 and/or CCL4 proteins in the sample.

14. The method of claim 13, wherein the measuring the amount of CCL3 and/or CCL4 protein is with an enzyme-linked immunosorbent assay (ELISA).

15. The method of claim 11, wherein the DLBCL is activated B cell-like (ABC) subtype of DLBCL.

16. The method of claim 11, wherein the ABC-DLBCL is characterized by a CD79B mutation or a CD79A mutation.

17. The method of claim 15, wherein the ABC-DLBCL is characterized by a mutation in MyD88, A20, or a combination thereof.

18. The method of claim 11, further comprising:
determining a post-administration expression level of CCL3 and/or CCL4 in a sample from the patient following administration of the ibrutinib; and
continuing treatment with ibrutinib if the post-administration expression of CCL3 and/or CCL4 is decreased by a predetermined amount relative to the pre-administration expression level of CCL3 and/or CCL4.

19. The method of claim 18, wherein the predetermined amount is a decrease in the level of CCL3 and/or CCL4 expression by 3%, 5%, 10%, 15%, 20%, 25%, 30%, 35%, 40%, 45%, 50%, 55%, 60%, 65%, 70%, 75%, 80%, 85%, 90%, 95%, 99% or greater following administration of ibrutinib.

20. The method of claim 18, wherein the predetermined amount is a decrease in the post-administration expression level of CCL3 and/or CCL4 to a level of expression of CCL3 and/or CCL4 in a human that does not have DLBCL.
